(19) United States
(12) Patent Application Publication
(10) Pub. No.: US 2003/0219774 A1
(43) Pub. Date: Nov. 27, 2003

(54) Novel human neurotransmitter transporter

(76) Inventors: Rahul Sharma, Gurnee, IL (US); Chandra S. Ramanathan, Wallingford, CT (US); Ryan Westphal, Chesire, CT (US); John N. Feder, Belle Mead, NJ (US); Liana M. Lee, North Brunswick, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/319,315

(22) Filed: Dec. 13, 2002

Related U.S. Application Data
(60) Provisional application No. 60/340,436, filed on Dec. 14, 2001.

(51) Int. Cl. C12Q 1/68; C07H 21/04; C07K 14/705; C12P 21/02; C12N 5/06

(52) U.S. Cl. 435/6; 435/69.1; 435/320.1; 435/325; 530/350; 536/23.5

(57) ABSTRACT

The invention provides a novel human orphan neurotransmitter transporter belonging to the family of Na⁺/Cl⁻ dependent transporters. Inventive HNTTBMY1 polypeptides and polynucleotides and methods for producing such polypeptides by recombinant techniques are disclosed. Further provided are methods for utilizing these polypeptides and polynucleotides in therapy and diagnostic assays for such. The transporter of the present invention is expressed highly in the amygdala brain subregion, which is known to be associated with affective disorders. The inventive transporter shares high homology with the rat orphan neurotransmitter transporter termed NTT4.

This application claims benefit to provisional application U.S. Serial No. 60/340,436 filed Dec. 14, 2001. The entire teachings of the referenced application are incorporated herein by reference.

### FIELD OF THE INVENTION

The present invention relates to neurotransmitter transporters. In particular, the invention relates to the Na⁺/Cl⁻ dependent neurotransmitter transporter gene family.

### BACKGROUND OF THE INVENTION

Chemical synapses are the primary mechanisms of information transfer in the central nervous system (CNS). Neurotransmitters are the messengers that mediate this information transfer. The release of the neurotransmitter from the presynaptic terminal into the synaptic gap followed by binding to the post-synaptic receptors, and the subsequent termination of the effects constitute the main steps in the synaptic transmission [Goodman and Gilman, The Pharmacological Basis of Therapeutics, Eds.—Goodman L. S. et al., 9^{th} Ed. McGraw Hill, ST, (1996)]. In both the central and peripheral nervous system, reliable neurotransmission depends on rapid termination of transmitter action following post-synaptic activation. In certain instances, this is achieved by metabolism of the neurotransmitter, as in the case of acetycholine and neuropeptides. However, in many cases, including catecholamines, serotonin, and some amino acids (e.g. GABA, glycine and glutamate), the neurotransmitter is efficiently removed into the presynaptic terminal or surrounding glial cells by neurotransmitter transporters (NTT's), which are membrane-bound polypeptides located in the plasma membrane. Inside the neuron, another family of transporters, vesicular transporters, help store the neurotransmitter in the vesical stores for further release [Neurotransmitter Transporters: Structure, Function and Regulation, Maarten and Reith, Humana Press, N.J., (1997); Methods in Enzymology, Neurotransmitter Transporters, Ed. Amara S. G., Volume 296. Academic Press, N.Y., (1998)].

Neurotransmitter transporters help maintain the tight control on neurotransmitter homeostasis needed for synaptic transmission, and are also the targets of action of a number of important psychoactive drugs. In particular, modulation of neurotransmitter transport enables synaptic transmission to be increased or decreased by altering the levels of neurotransmitter in the synaptic cleft, and blockade of transport is an established approach to the treatment of psychiatric and neurological illness. The norepinephrine and serotonin NTT's are the main targets of the anti-depressant drugs, for example.

Neurotransmitters are transported by NTF's up the concentration gradient by linking the process to the transport of ions, such as sodium, down the electrochemical gradient. As such, NTT's belong to a larger superfamily of mammalian transporters that are coupled to the co-transport of H⁺, Na⁺, and Cl⁻ and/or to the counter-transport of K⁺ or OH⁻ [Hediger M. A. et al., “Mammalian Ion-coupled Solute Transporters,” J. Physiol., 482:7S-17S, (1995)]. Generally, the energy required for transporting the neurotransmitter across the plasma membrane of cells is provided via Na⁺/K⁺ ATPase, which maintains the ion gradient.

All transporters, including NTT's can be divided into subfamilies based on criteria such as their sequence homology, phylogenetic grouping, mode of transport, energy coupling mechanism or substrates transported by them. One classification divides the larger mammalian ion-coupled solute transporter family into the Na⁺ coupled transporter family, the Na⁺ and Cl⁻ coupled transporter family, the Na⁺ and K⁺ dependent transporter family, and the H⁺ coupled transporter family [Neurotransmitter Transporters: Structure, Function and Regulation, Maarten and Reith, Humana Press, N.J., (1997); Methods in Enzymology, Neurotransmitter Transporters, Ed. Amara S. G., Volume 296. Academic Press, N.Y., 1998); Masson, J, et al., Pharmacol. Review, 51(3):439-464, (1999); Frazer, A, Gerhardt, G. A. and Daws, L. C., Int'l. J. Neuropsychopharm, 2(4):305-320, (1999)].

Recently, cDNA's encoding more than ten different neurotransmitter transporters have been cloned and sequenced which belong to the Na⁺ and Cl⁻ coupled transporter family. Transporters in this family include those for norepinephrine [Pacholczyk, T., et al., Nature 350: 350-354 (1991)], serotonin [Lesch, et al. J. Neural Transm. 91, 67-73 (1993)], dopamine [Giros, B., et al. Molecular Pharmacology 42(3), 383-390 (1992)], glycine [Lin et al. J. Biol Chem., 268, 22802-22808 (1992)], GABA [Guastella, J. et al. Science 249: 1303-1306 (1990)], betaine [Lopez-Corcuera, et al. J. Biol. Chem. 267 (25), 17491-17493 (1992)], taurine/β-alanine [Liu, Q. R. et al. Proc. Natl. Acad. Sci. USA, (1992)], L-proline [Fremeau, Jr., R. T., et al., Neuron, 8: 915-926 (1992)], and creatine [Guimbal, C. and Kilimann, M. W. J. Biol. Chem. 268 (12), 8418-8421 (1993)]. Each of these transporters has been found in the brain and nearly all have substrates that are either neuroregulators, omoregulators, or both, which reinforces the concept that molecules with similar structures often have similar functions.

Members of the Na⁺/Cl⁻ dependent NTT's typically share a number of common features. For example, members of the family typically exhibit from about 40 to about 60% homology to one another. These transporters are single-chain polypeptides from 500-600 amino acids [Iversen L., Molecular Psychiatry, 5(4):357-362, (2000)]. They have twelve predicted transmembrane domains, intracellular N- and C-terminal domains, potential sites for glycosylation in extracellular domains, and dependence on sodium for transport activity. Moreover, they have putitive protein kinase C phosphorylation sites, and in some cases, putitive cAMP-dependent protein kinase A phosphorylation sites, which may represent an important regulatory mechanism for their activity by second messengers [Masson, J, et al., Pharmacol. Review, 51(3):439-464, (1999)].

In addition to the classical Na⁺/Cl⁻ dependent transporters described above, several atypical Na⁺/Cl⁻ dependent transporters have been cloned that exhibit significant amino acid identity with the classical transporters, but whose endogonous substrates have not yet been identified. They have been termed “orphan” because the substrates transported by them are as yet known. They,have some distinguishing features of their own. They exhibit about 20 to 30% sequence homology to the “classical” Na⁺/Cl⁻ dependent NTT's [Masson, J, et al., Pharmacol. Review, 51(3):439-464, (1999); Frazer, A, Gerhardt, G. A. and Daws, L. C., Int'l. J. Neuropsychopharm., 2(4):305-320, (1999)], however orphan transporters share about 50-60% homology with each other. Their common structural features suggest functional similarities. These include an additional glycosylation site on the fourth extracellular loop. In addition, many of the orphan NTT's identified today are reported to have enlarged fourth and sixth extracellular loops. Examples of orphan transporters found in the central nervous system include Rxt1 (also referred to rat xt1 or NTT4), as well as v7-3 and v7-3-2. Furthermore, peripheral tissue specific orphan transporters have been identified to include ROSIT (renal osmotic stress induced transporter), rB21a, and NTT5. Both NTT5 and v7-3 have been shown to have large extracellular loops between transmembrane regions 3 and 4 [Farmer, M. K., et al., Genomics, 70:241-252, (2000)]. It has been suggested that identifying the substrates of “orphan” transporters could reveal previously undescribed neurotransrnitter systems.

Many long lasting behavioral differences have heritabilities of 30% or more. The availability of the human genome sequences and identification of single nucleotide polymorphisms (SNP's) is making it possible to determine the genetic variation (heterozygosity), in the population. The study of heterozygosity in the NTT genes (including orphan NTT genes) and their association with various diseases and disease susceptibilities enable new methods of diagnosis, prevention and treatment of a variety of these disorders. For example, it has been observed that the 480-base pair (bp) DAT1 allele of the Na⁺/Cl⁻ dependent dopamine transporter gene termed DAT is associated with ADHD. A 40-bp tandem repeat sequence (VNTR, Variable Number Tandem Repeat) in the 3′ untranslated region of the human DAT1 gene has been linked to altered DAT availability in the striatum [Cravchik, A, and Goldman, D., Arch. Gen. Psychiatry, 57(12):1105-1114, (2000)]. In spino-cerebellar ataxia of type 1, the amount of DAT is reduced in striatal axon terminals [Masson, J, et al., Pharmacol. Review, 51(3):439-464, (1999)]. The gene coding for the human SERT (a Na⁺/Cl⁻ dependent serotonin transporter) is located on chromosome 17q11.2. No allelic variation has been observed in the coding region of the gene in patients with affective disorders. However, multiple polymorphisms are found in the 5′ flanking region and in the second intron. The allelic variants at the second intron have been observed to be associated with bipolar and unipolar disorders. The two variants in the 5′ region are linked with different levels of SERT expression. The variant with the lowest transcription rate seems to be more frequent in alcoholics, in suicidal individuals, and in persons with an anxious personality trait [Masson, J, et al., “Neurotransmitter Transporters in the Central Nervous System,” Pharmacol. Review, 51(3):439-464, (1999)]:

The recent cloning of genes encoding neurotransmitter transporters is increasing the understanding of the role of transport proteins in nervous system health and disease. The availability of cloned transporters provides the opportunity to define the pharmacological profiles of specific gene products, to map their patterns of distribution, and to make correlations with in vivo observations to better understand their biological functions. In particular, identification of mutations responsible for genetic disorders or other traits is possible.

Furthermore, the discovery of nucleic acid sequences and polypeptide sequences of novel neurotransmitter transporters enables one to identify molecules that affect the transport activity associated with NTT's and to treat mammals afflicted with diseases associated with a lesion of an NTT, such as that characterized by an alteration in sequence or expression of the NTT. There is a particular need for discovery of novel neurotransmitter transporters from the human species.

### SUMMARY OF THE INVENTION

The present invention is based on the discovery of a cDNA molecule, designated clone HNTTBMY1, encoding a novel human orphan neurotransmitter transporter (NTT). In particular, the inventive transporter appears to be a Na⁺/Cl⁻ dependent neurotransmitter transporter. SEQ ID No: 2 shows the nucleotide sequence of the inventive transporter and SEQ ID NO: 1 represents the deduced amino acid sequence of this transporter. SEQ ID NO: 3 represents a 5′ untranslated nucleotide sequence upstream of the nucleotide sequence encoding the inventive transporter and corresponds to polynucleotides 1 to 379 of SEQ ID NO: 2.

In particular, the invention provides an isolated or recombinant polynucleotide encoding a human neurotransmitter transporter protein including the nucleotide sequence of SEQ ID NO: 2 or a fragment or mutant form thereof.

Further provided is an isolated or recombinant polynucleotide including the 5′ untranslated nucleotide sequence upstream of a nucleotide sequence encoding a human neurotransmitter transporter, wherein the 5′ untranslated sequence includes the nucleotide sequence of SEQ ID NO: 3 or a fragment or mutant form thereof.

Also encompassed by the invention is a cloned mutant nucleic acid form of a human neurotransmitter transporter, wherein the mutant form mediates or modulates a transmitter transporter activity of the protein of SEQ ID NO: 1.

Further provided is a substantially pure oligonucleotide or primer, the oligonucleotide or primer comprising a region of nucleotide sequence capable of hybridizing under stringent conditions to at least about 12 consecutive nucleotides of a sense sequence or an antisense sequence of a human polynucleotide including the nucleotide sequence of SEQ ID NO: 2 or an analog or mutant form thereof.

In addition, the invention provides a substantially pure oligonucleotide or primer, the oligonucleotide or primer comprising a region of nucleotide sequence capable of hybridizing under stringent conditions to at least about 12 consecutive nucleotides of sense or antisense sequence of a non-coding nucleotide sequence 5′ or 3′ of the coding sequence for the polypeptide of SEQ ID NO: 1.

Also provided by the invention is an isolated or recombinant human polypeptide, comprising the amino acid sequence of SEQ ID NO: 1 or a fragment or mutant form thereof.

The invention further encompasses a fusion protein comprising a first polypeptide of SEQ ID NO: 1 or a fragment or mutant form thereof, and a second polypeptide having an amino acid sequence unrelated to the amino acid sequence of the first polypeptide.

In addition, the invention provides an expression vector capable of expressing in a host cell a polypeptide including the amino acid sequence of SEQ ID NO: 1 or a fragment or mutant form thereof.

Further encompassed by the invention is a monoclonal antibody that binds specifically to an antigenic determinant in a human neurotransmitter transporter having the amino acid sequence of SEQ ID NO: 1 or mutant forms thereof.

The invention further provides for a transgenic animal having cells which harbor a transgene encoding a human neurotransmitter transporter of SEQ ID NO: 1 or a fragment thereof. Further provided is a transgenic animal having cells in which a gene encoding a human neurotransmitter transporter of SEQ ID NO: 1 or a fragment thereof is disrupted.

The present invention provides several methods of use of the polynucleotides and polypeptides of the present invention. For example, the invention provides a method of mediating or modulating a neurotransmitter transporter activity of a neurotransmitter transporter, the method including introducing into a cell an isolated or recombinant human neurotransmitter transporter protein comprising the amino acid sequence of SEQ ID NO: 1 or a fragment, analog or mutant form thereof.

Further encompassed by the invention is a method of modulating a neurotransmitter transporter activity of a neurotransmitter transporter, the method including introducing into a cell an agonist of a nucleic acid form encoding a human neurotransmitter transporter protein of SEQ ID NO: 1 or an agonist of amino acids encoded by the nucleic acid form.

In addition, the invention provides a method of modulating the transmitter transporter activity of a neurotransmitter transporter, the method including introducing into a cell an antagonist of a nucleic acid form encoding a human polpypeptide of SEQ ID NO: 1 or an antagonist of amino acids encoded by the nucleic acid form.

Another aspect of the invention features a method for identifying therapeutic agents that inhibit, potentiate, or mimic the ability of a human neurotransmitter transporter protein to transport a neurotransmitter, the method including: (a) treating a cell with an effective amount of at least one candidate so as to alter the transmitter transporter activity associated with the amino acid sequence of SEQ ID NO: 1 or a fragment or mutant form thereof; and (b) measuring the effect of the candidate on the cell.

The invention further provides a method of determining if a patient is at risk for a disorder or has a disorder, the method including detecting, in a patient specimen, the presence or absence of a lesion characterized by an alteration in sequence, expression, post-translational modification, or a combination thereof of a human neurotransmitter transporter including the amino acid sequence of SEQ ID NO: 1 or a nucleic acid form including the nucleotide sequence of SEQ ID NO: 2 or SEQ ID NO: 3.

The invention further provides a method for detecting a nucleic acid form of a human neurotransmitter transporter in a biological sample, the method including: (a) hybridizing a nucleic acid form including the nucleotide sequence of SEQ ID NO: 2 or SEQ ID NO: 3 or a fragment or mutant form of either to nucleic acid material of a biological sample, thereby forming a hybridization complex; and (b) detecting the hybridization complex, wherein the presence of the complex correlates with the presence of a nucleic acid form of the transporter in the biological sample.

In another embodiment, the invention provides a method for detecting a human neurotransmitter transporter including the amino acid sequence of SEQ ID No: 1 or a fragment or mutant form thereof, the method including the step of performing an immunoassay on a biological sample.

Also provided is a method for detecting a nucleic acid form of a human neurotransmitter transporter including the steps of: (a) amplifying a nucleic acid form including SEQ ID NO: 2 or SEQ ID NO: 3 or a fragment or mutant form of either in a biological sample; and (b) detecting sequence alterations in the biological sample.

Furthermore, the invention provides a method of diagnosing a neurotransmitter transporter related condition in at least one patient specimen by comparing RNA profiles of the specimens with a control. The method includes the steps of: (a) obtaining a sample of ribonucleic acids from each of the patient specimens; (b) generating a population of labeled nucleic acids for each of the patient samples from the sample of ribonucleic acids; (c) hybridizing the labeled nucleic acids for each of the patient samples to an array of nucleic acid molecules stably associated with a surface of a substrate to produce a hybridization pattern for each of the patient specimens; the stably associated nucleic acid molecules including SEQ ID NO: 2 or complements thereof or fragments or mutants of either; and (d) comparing hybridization patterns for each of the patient samples to a control.

The invention also provides a method for producing a polypeptide according to the present invention. For example, the invention provides a method for producing a human neurotransmitter transporter of SEQ ID NO: 2 or a fragment or mutant form thereof, the method including the steps of: (a) culturing a host cell transfected with an expression vector capable of expressing in the cell the transporter of SEQ ID NO: 2 or a fragment or mutant form thereof under conditions suitable for the expression of the transporter or fragment; and (b) recovering the transporter or fragment from the host cell culture.

The invention further relates to a polynucleotide encoding a polypeptide fragment of SEQ ID NO: 1, or a polypeptide fragment encoded by the cDNA sequence included in the deposited clone, which is hybridizable to SEQ ID NO: 2.

The invention further relates to a polynucleotide encoding a polypeptide domain of SEQ ID NO: 1 or a polypeptide domain encoded by the cDNA sequence included in the deposited clone, which is hybridizable to SEQ ID NO: 2.

The invention further relates to a polynucleotide encoding a polypeptide epitope of SEQ ID NO: 1 or a polypeptide epitope encoded by the cDNA sequence included in the deposited clone, which is hybridizable to SEQ ID NO: 2.

The invention further relates to a polynucleotide encoding a polypeptide of SEQ ID NO: 1 or the cDNA sequence included in the deposited clone, which is hybridizable to SEQ ID NO: 2, having biological activity.

The invention further relates to a polynucleotide which is a variant of SEQ ID NO: 2.

The invention further relates to a polynucleotide which is an allelic variant of SEQ ID NO: 2.

The invention further relates to a polynucleotide which encodes a species homologue of the SEQ ID NO: 1.

The invention further relates to a polynucleotide which represents the complimentary sequence (antisense) of SEQ ID NO: 2.

The invention further relates to a polynucleotide capable of hybridizing under stringent conditions to any one of the polynucleotides specified herein, wherein said polynucleotide does not hybridize under stringent conditions to a nucleic acid molecule having a nucleotide sequence of only A residues or of only T residues.

The invention further relates to an isolated nucleic acid molecule of SEQ ID NO: 1, wherein the polynucleotide fragment comprises a nucleotide sequence encoding a neurotransmitter transporter.

The invention further relates to an isolated nucleic acid molecule of SEQ ID NO: 2 wherein the polynucleotide fragment comprises a nucleotide sequence encoding the sequence identified as SEQ ID NO: 1 or the polypeptide encoded by the cDNA sequence included in the deposited clone, which is hybridizable to SEQ ID NO: 2.

The invention further relates to an isolated nucleic acid molecule of of SEQ ID NO: 2, wherein the polynucleotide fragment comprises the entire nucleotide sequence of SEQ ID NO: 2 or the cDNA sequence included in the deposited clone, which is hybridizable to SEQ ID NO: 2.

The invention further relates to an isolated nucleic acid molecule of SEQ ID NO: 2, wherein the nucleotide sequence comprises sequential nucleotide deletions from either the C-terminus or the N-terminus.

The invention further relates to an isolated polypeptide comprising an amino acid sequence that comprises a polypeptide fragment of SEQ ID NO: 1 or the encoded sequence included in the deposited clone.

The invention further relates to a polypeptide fragment of SEQ ID NO: 1 or the encoded sequence included in the deposited clone, having biological activity.

The invention further relates to a polypeptide domain of SEQ ID NO: 1 or the encoded sequence included in the deposited clone.

The invention further relates to a polypeptide epitope of SEQ ID NO: 1 or the encoded sequence included in the deposited clone.

The invention further relates to a full length protein of SEQ ID NO: 1 or the encoded sequence included in the deposited clone.

The invention further relates to a variant of SEQ ID NO: 1.

The invention further relates to an allelic variant of SEQ ID NO: 1. The invention further relates to a species homologue of SEQ ID NO: 1.

The invention further relates to the isolated polypeptide of of SEQ ID NO: 1, wherein the full length protein comprises sequential amino acid deletions from either the C-terminus or the N-terminus.

The invention further relates to an isolated antibody that binds specifically to the isolated polypeptide of SEQ ID NO: 1.

The invention further relates to a method for preventing, treating, or ameliorating a medical condition, comprising administering to a mammalian subject a therapeutically effective amount of the polypeptide of SEQ ID NO: 1 or the polynucleotide of SEQ ID NO: 2.

The invention further relates to a method of diagnosing a pathological condition or a susceptibility to a pathological condition in a subject comprising the steps of (a) determining the presence or absence of a mutation in the polynucleotide of SEQ ID NO: 2; and (b) diagnosing a pathological condition or a susceptibility to a pathological condition based on the presence or absence of said mutation.

The invention further relates to a method of diagnosing a pathological condition or a susceptibility to a pathological condition in a subject comprising the steps of (a) determining the presence or amount of expression of the polypeptide of of SEQ ID NO: 1 in a biological sample; and diagnosing a pathological condition or a susceptibility to a pathological condition based on the presence or amount of expression of the polypeptide.

The invention further relates to a method for identifying a binding partner to the polypeptide of SEQ ID NO: 1 comprising the steps of (a) contacting the polypeptide of SEQ ID NO: 1 with a binding partner; and (b) determining whether the binding partner effects an activity of the polypeptide.

The invention further relates to a gene corresponding to the cDNA sequence of SEQ ID NO: 2.

The invention further relates to a method of identifying an activity in a biological assay, wherein the method comprises the steps of expressing SEQ ID NO: 2 in a cell, (b) isolating the supernatant; (c) detecting an activity in a biological assay; and (d) identifying the protein in the supernatant having the activity.

The invention further relates to a process for making polynucleotide sequences encoding gene products having altered activity selected from the group consisting of SEQ ID NO: 1 activity comprising the steps of (a) shuffling a nucleotide sequence of SEQ ID NO: 2, (b) expressing the resulting shuffled nucleotide sequences and, (c) selecting for altered activity selected from the group consisting of SEQ ID NO: 1 activity as compared to the activity selected from the group consisting of SEQ ID NO: 1 activity of the gene product of said unmodified nucleotide sequence.

The invention further relates to a shuffled polynucleotide sequence produced by a shuffling process, wherein said shuffled DNA molecule encodes a gene product having enhanced tolerance to an inhibitor of any one of the activities selected from the group consisting of SEQ ID NO: 1 activity.

The invention further relates to a method for preventing, treating, or ameliorating a medical condition with the polypeptide provided as SEQ ID NO: 1, in addition to, its encoding nucleic acid, wherein the medical condition is a neural disorder.

The invention further relates to a method of identifying a compound that modulates the biological activity of HNTTBMY1, comprising the steps of, (a) combining a candidate modulator compound with HNTTBMY1 having the sequence set forth in one or more of SEQ ID NO: 1; and measuring an effect of the candidate modulator compound on the activity of HNTTBMY1.

The invention further relates to a method of identifying a compound that modulates the biological activity of a GPCR, comprising the steps of, (a) combining a candidate modulator compound with a host cell expressing HNTTBMY1 having the sequence as set forth in SEQ ID NO: 1; and, (b) measuring an effect of the candidate modulator compound on the activity of the expressed HNTTBMY1.

The invention further relates to a method of identifying a compound that modulates the biological activity of HNTTBMY1, comprising the steps of, (a) combining a candidate modulator compound with a host cell containing a vector described herein, wherein HNTTBMY1 is expressed by the cell; and, (b) measuring an effect of the candidate modulator compound on the activity of the expressed HNTTBMY1.

The invention further relates to a method of screening for a compound that is capable of modulating the biological activity of HNTTBMY1, comprising the steps of: (a) providing a host cell described herein; (b) determining the biological activity of HNTTBMY1 in the absence of a modulator compound; (c) contacting the cell with the modulator compound; and (d) determining the biological activity of HNTTBMY1 in the presence of the modulator compound; wherein a difference between the activity of HNTTBMY1 in the presence of the modulator compound and in the absence of the modulator compound indicates a modulating effect of the compound.

The invention further relates to a compound that modulates the biological activity of human HNTTBMY1 as identified by the methods described herein.

The invention further relates to a polynucleotide of further comprising a 5′ or 3′ regulatory sequence.

The invention further relates to a fusion protein comprising a first polypeptide of SEQ ID NO: 1 or a fragment or mutant form thereof, and a second polypeptide having an amino acid sequence unrelated to the amino acid sequence of said first polypeptide. The invention further relates to said fusion protein wherein said second polypeptide functions as a detectable label for detecting the presence of said fusion protein or as a matrix-binding domain for immobilizing said fusion protein.

The invention further relates to a substantially pure oligonucleotide or primer, said oligonucleotide or primer comprising a region of nucleotide sequence capable of hybridizing under stringent conditions to at least about 12 consecutive nucleotides of a sense sequence or an antisense sequence of a human polynucleotide comprising the nucleotide sequence of SEQ ID NO: 2 or an analog or mutant form thereof. The invention further relates to said oligonucleotide, wherein the oligonucleotide further comprises a detectable label attached thereto.

The invention further relates to a substantially pure oligonucleotide or primer, said oligonucleotide or primer comprising a region of nucleotide sequence capable of hybridizing under stringent conditions to at least about 12 consecutive nucleotides of sense or antisense sequence of a non-coding nucleotide sequence 5′ or 3′ of the coding sequence for the polypeptide of SEQ ID NO: 1. The invention further relates to said oligonucleotide, wherein the oligonucleotide further comprises a detectable label attached thereto. The invention further relates to said oligonucleotide, wherein the 5′ non-coding sequence comprises the nucleotide sequence of SEQ ID NO: 3.

The invention further relates to a method of mediating or modulating a neurotransmitter transporter activity of a neurotransmitter transporter, the method comprising introducing into a cell an isolated or recombinant human neurotransmitter transporter protein comprising the amino acid sequence of SEQ ID NO: 1 or a fragment, analog or mutant form thereof. The invention further relates to said method, wherein the transporter protein, fragment or mutant form thereof is introduced into the cell by expressing in the cell a nucleic acid molecule that encodes the protein or fragment. The invention further relates to said method wherein the cell is derived from brain or spinal cord. The invention further relates to said method, wherein the cell is derived from a brain subregion selected from the group consisting of amygdala, caudate nucleus, cerebellum, corpus callosum, hippocampus, substantia nigra, thalamus, and combinations thereof.

The invention further relates to A method of modulating a neurotransmitter transporter activity of a neurotransmitter transporter, the method comprising introducing into a cell an agonist of a nucleic acid form encoding a human neurotransmitter transporter protein of SEQ ID NO: 1 or an agonist of amino acids encoded by the nucleic acid form. The invention further relates to said method, wherein the cell is derived from brain or spinal cord. The invention further relates to said method, wherein the cell is derived from a brain subregion selected from the group consisting of amygdala, caudate nucleus, cerebellum, corpus callosum, hippocampus, substantia nigra, thalamus, and combinations thereof.

The invention further relates to a method of modulating the transmitter transporter activity of a neurotransmitter transporter, the method comprising introducing into a cell an antagonist of a nucleic acid form encoding a human polpypeptide of SEQ ID NO: 1 or an antagonist of amino acids encoded by the nucleic acid form. The invention further relates to said method, wherein the cell is derived from brain or spinal cord. The invention further relates to said method, wherein the cell is derived from a brain subregion selected from the group consisting of amygdala, caudate nucleus, cerebellum, corpus callosum, hippocampus, substantia nigra, thalamus, and combinations thereof.

The invention further relates to a method for identifying therapeutic agents that inhibit, potentiate, or mimic the ability of a human neurotransmitter transporter protein to transport a neurotransmitter, the method comprising: (a) treating a cell with an effective amount of at least one candidate so as to alter the transmitter transporter activity associated with the amino acid sequence of SEQ ID NO: 1 or a fragment or mutant form thereof; and (b) measuring the effect of the candidate on the cell.

The invention further relates to a method of determining if a patient is at risk for a disorder or has a disorder, the method comprising detecting, in a patient specimen, the presence or absence of a lesion characterized by an alteration in sequence, expression, post-translational modification, or a combination thereof of a human neurotransmitter transporter comprising the amino acid sequence of SEQ ID NO: 1 or a nucleic acid form comprising the nucleotide sequence of SEQ ID NO: 2 or SEQ ID NO: 3. The invention further relates to said method, wherein the presence or absence of a lesion is characterized by: (a) a mutation of a gene encoding the protein of SEQ ID NO: 1 or a homologue thereof; (b) a mis-expression of said gene; (c) an altered neurotransmitter transporter activity of the protein of SEQ ID NO: 1; (d) a polymorphism in the 5′ untranslated region of SEQ ID NO: 3 and combinations thereof. The invention further relates to said method, wherein the disorder is selected from the group consisting of affective disorders, psychotic disorders, neurological metabolic disorders, immune-related disorders, hypotension, hypertension, endocrinal diseases, growth disorders, neuropathic pain, obesity, anorexia, bulimia, Parkinson's disease, dementias, and combinations thereof.

The invention further relates to a method of diagnosing a neurotransmitter transporter related condition in at least one patient specimen by comparing RNA profiles of said specimens with a control comprising the steps of: (a) obtaining a sample of ribonucleic acids from each of the patient specimens; (b) generating a population of labeled nucleic acids for each of the patient samples from said sample of ribonucleic acids; (c) hybridizing the labeled nucleic acids for each of the patient samples to an array of nucleic acid molecules stably associated with a surface of a substrate to produce a hybridization pattern for each of the patient specimens; said stably associated nucleic acid molecules including SEQ ID NO: 2 or complements thereof or fragments or mutants of either; and (d) comparing hybridization patterns for each of the patient samples to a control.

The invention further relates to a transgenic animal having cells which harbor a transgene encoding a human neurotransmitter transporter of SEQ ID NO: 1 or a fragment thereof.

The invention further relates to a transgenic animal having cells in which a gene encoding a human neurotransmitter transporter of SEQ ID NO: 1 or a fragment thereof is disrupted.

The invention further relates to a method for preventing, treating, or ameliorating a medical condition with the polypeptide provided as SEQ ID NO: 1, in addition to, its encoding nucleic acid, or a modulator thereof, wherein the medical condition is a behavioral disorder; memory disorders; cognitive disorders; disorders associated with aberrant serotonin expression and/or activity; anxiety, fear, depression, sleep, pain, disorders associated with aberrant maintenance of an attentive or alert state; attention deficit disorders; disorders affecting the ‘reward center’ of the brain; disorders affecting the synthesis, and/or effecting the release of neurotransmitters such as dopamine, opioid peptides, serotonin, GABA, and glutamate; addictive disorders; homeostatic disorders; neuroendocrine disorders; disorders affecting the establishment of long term potentiation; circadian rhythm disorders; disorders associated with the establishment of aberrant sleep/wake cycles; dopaminergic functional disorders; neuronal transmission system disorders, and pain.

The invention further relates to a method of diagnosing a pathological condition or a susceptibility to a pathological condition in a subject comprising the steps of (a) determining the presence or amount of expression of the polypeptide of of SEQ ID NO: 2 in a biological sample; (b) and diagnosing a pathological condition or a susceptibility to a pathological condition based on the presence or amount of expression of the polypeptide relative to a control, wherein said condition is a member of the group consisting of behavioral disorders; memory disorders; cognitive disorders; disorders associated with aberrant serotonin expression and/or activity; anxiety, fear, depression, sleep, pain, disorders associated with aberrant maintenance of an attentive or alert state; attention deficit disorders; disorders affecting the ‘reward center’ of the brain; disorders affecting the synthesis, and/or effecting the release of neurotransmitters such as dopamine, opioid peptides, serotonin, GABA, and glutamate; addictive disorders; homeostatic disorders; neuroendocrine disorders; disorders affecting the establishment of long term potentiation; circadian rhythm disorders; disorders associated with the establishment of aberrant sleep/wake cycles; dopaminergic functional disorders; neuronal transmission system disorders, and pain.

The present invention also relates to an isolated polynucleotide consisting of a portion of the human HNTTBMY1 gene consisting of at least 8 bases, specifically excluding Genbank Accession Nos. BU613681; BB622586; AU080032; BG295119; AU123498; BQ180354; CA327151; BU052772; CA318436; BU058760; CA360178; BI872482; BI681242; BB661608; and/or BQ831895.

The present invention also relates to an isolated polynucleotide consisting of a nucleotide sequence encoding a fragment of the human HNTTBMY1 protein, wherein said fragment displays one or more functional activities specifically excluding Genbank Accession Nos. BU613681; BB622586; AU080032; BG295119; AU123498; BQ180354; CA327151; BU052772; CA318436; BU058760; CA360178; BI872482; BI681242; BB661608; and/or BQ831895.

The present invention also relates to the polynucleotide of SEQ ID NO: 2 consisting of at least 10 to 50 bases, wherein said at least 10 to 50 bases specifically exclude the polynucleotide sequence of Genbank Accession Nos. BU613681; BB622586; AU080032; BG295119; AU123498; BQ180354; CA327151; BU052772; CA318436; BU058760; CA360178; BI872482; BI681242; BB661608; and/or BQ831895.

The present invention also relates to the polynucleotide of SEQ ID NO: 2 consisting of at least 15 to 100 bases, wherein said at least 15 to 100 bases specifically exclude the polynucleotide sequence of Genbank Accession Nos. BU613681; BB622586; AU080032; BG295119; AU123498; BQ180354; CA327151; BU052772; CA318436; BU058760; CA360178; BI872482; BI681242; BB661608; and/or BQ831895.

The present invention also relates to the polynucleotide of SEQ ID NO: 2 consisting of at least 100 to 1000 bases, wherein said at least 100 to 1000 bases specifically exclude the polynucleotide sequence of Genbank Accession Nos. BU613681; BB622586; AU080032; BG295119; AU123498; BQ180354; CA327151; BU052772; CA318436; BU058760; CA360178; BI872482; BI681242; BB661608; and/or BQ831895.

The present invention also relates to an isolated polypeptide fragment of the human HNTTBMY1 protein, wherein said polypeptide fragment does not consist of the polypeptide encoded by the polynucleotide sequence of Genbank Accession Nos. BU613681; BB622586; AU080032; BG295119; AU123498; BQ180354; CA327151; BU052772; CA318436; BU058760; CA360178; B1872482; BI681242; BB661608; and/or BQ831895.

### BRIEF DESCRIPTIONS OF THE DRAWINGS

FIGS. 1A-C show the polynucleotide sequence (SEQ ID NO: 2) and deduced amino acid sequence (SEQ ID NO: 1) of the human orphan neurotransmitter transporter, HNTTBMY1, of the present invention. The standard one-letter abbreviation for amino acids is used to illustrate the deduced amino acid sequence. The polynucleotide sequence contains a sequence of 2572 nucleotides (SEQ ID NO: 2), encoding a polypeptide of 727 amino acids (SEQ ID NO: 1). An analysis of the HNTTBMY1 polypeptide determined that it comprised the following features: twelve transmembrane domains (TM1 to TM12) located from about amino acid 69 to about amino acid 90 (TM1; SEQ ID NO: 35); from about amino acid 96 to about amino acid 118 (TM2; SEQ ID NO: 36); from about amino acid 139 to about amino acid 163 (TM3; SEQ ID NO: 37); from about amino acid 227 to about amino acid 246 (TM4; SEQ ID NO: 38); from about amino acid 252 to about amino acid 284 (TM5; SEQ ID NO: 39); from about amino acid 301 to about amino acid 320 (TM6; SEQ ID NO: 40); from about amino acid 338 to about amino acid 357 (TM7; SEQ ID NO: 41); from about amino acid 460 to about amino acid 486 (TM8; SEQ ID NO: 42); from about amino acid 495 to about amino acid 516 (TM9; SEQ ID NO: 43); from about amino acid 526 to about amino acid 546 (TM10; SEQ ID NO: 44); from about amino acid 578 to about amino acid 598 (TM11; SEQ ID NO: 45); and/or from about amino acid 618 to about amino acid 638 (TM12; SEQ ID NO: 46) of SEQ ID NO: 1 (FIGS. 1A-C) represented by double underlining. The transmembrane regions within the amino acid sequence (SEQ ID NO: 1) of the human orphan neurotransmitter transporter of the present invention were predicted using the TMPRED program (prediction score above 500).

FIG. 2 shows a local alignment of the amino acid sequence (SEQ ID NO: 1) of the human orphan neurotransmitter transporter of the present invention against the amino acid sequence (SEQ ID NO: 30) of the target Pfam model (T) (SNS PF 00209 sodium: neurotransmitter symporter family).

FIG. 3, comprising FIG. 3A through 3G, is a global alignment of the amino acid sequence (SEQ ID NO: 1) of the orphan neurotransmitter transporter of the present invention (from the N-terminus through the C-terminus of the protein) with other orphan neurotransmitter transporter sequences (SEQ ID NOS: 12-29). Sequence identity is indicated by black highlighting; sequence similarity is indicated by gray highlighting. The GCP pileup program was used to generate the alignment.

FIG. 4 depicts a hydrophilicity plot indicating the transmembrane region prediction for the human neurotransmitter transporter of the present invention.

FIG. 5 is a graph showing the expression profiling in various human tissues for the orphan neurotransmitter transporter of the present invention.

FIG. 6 is a graph showing the expression profiling in particular human brain subregions for the orphan neurotransmitter transporter of the present invention.

FIG. 7 shows an expanded expression profile of the novel human neurotransmitter transporter, HNTTBMY1. The figure illustrates the relative expression level of HNTTBMY1 amongst various mRNA tissue sources. As shown, the HNTTBMY1 polypeptide was expressed predominately in the nervous system, specifically throughout the cortex. Expression of HNTTBMY1 was also significantly expressed in the hippocampus, cerebellum, the pituitary, the locus coeruleus, the dorsal raphe nucleus, the nucleus accumbens, the substantia nigra, the pineal gland, the hypothalamus, the caudate, the amygdala, and to a lesser extent in other regions as shown. Transcripts for HNTTBMY1 are also found in low relative abundance in the spinal cord and DRG. Expression data was obtained by measuring the steady state HNTTBMY1 mRNA levels by quantitative PCR using the PCR primer pair provided as SEQ ID NO: 96 and 97, and Taqman probe (SEQ ID NO: 98) as described in Example 8 herein.

Table I provides a table illustrating the percent identity and percent similarity between the HNTTBMY1 polypeptide of the present invention with other neurotransmitter transporters.

Table II provides a summary of the novel polypeptides and their encoding polynucleotides of the present invention.

Table III illustrates the preferred hybridization conditions for the polynucleotides of the present invention. Other hybridization conditions may be known in the art or are described elsewhere herein.

Table IV provides a summary of various conservative substitutions encompassed by the present invention.

### DETAILED DESCRIPTION OF THE INVENTION

The nucleotide sequence of the cDNA encoding the polypeptide of the invention is represented by SEQ ID NO: 2. The amino acid sequence of the polypeptide of the invention is represented by SEQ ID NO: 1. The inventive transporter (HNTTBMY1) is an orphan transporter that appears to belong to the Na⁺/Cl⁻ dependent family of neurotransmitter transporters.

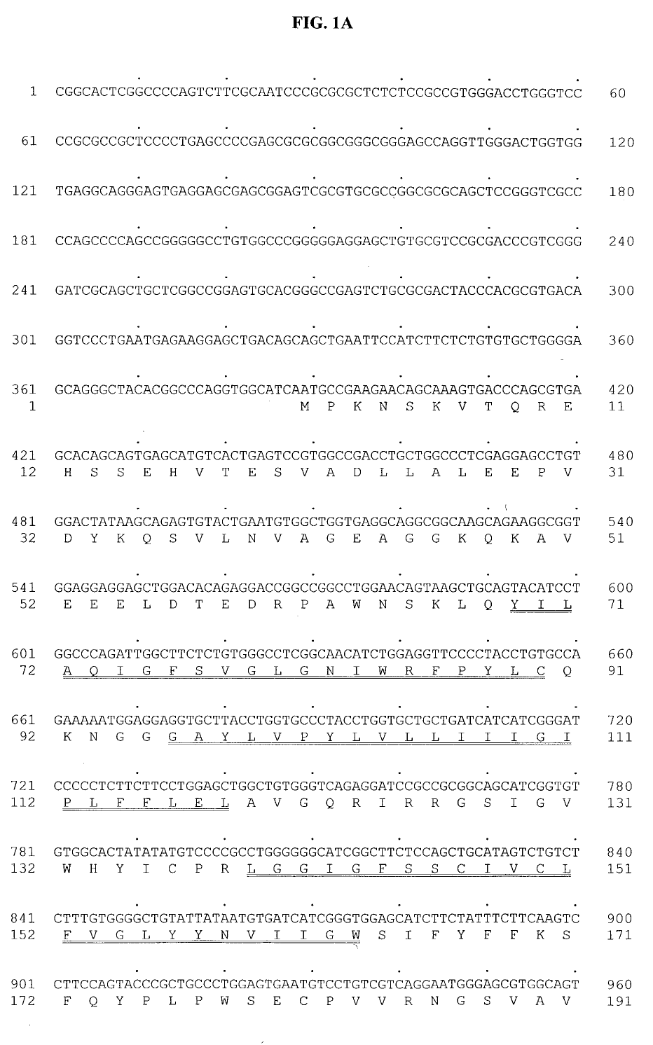

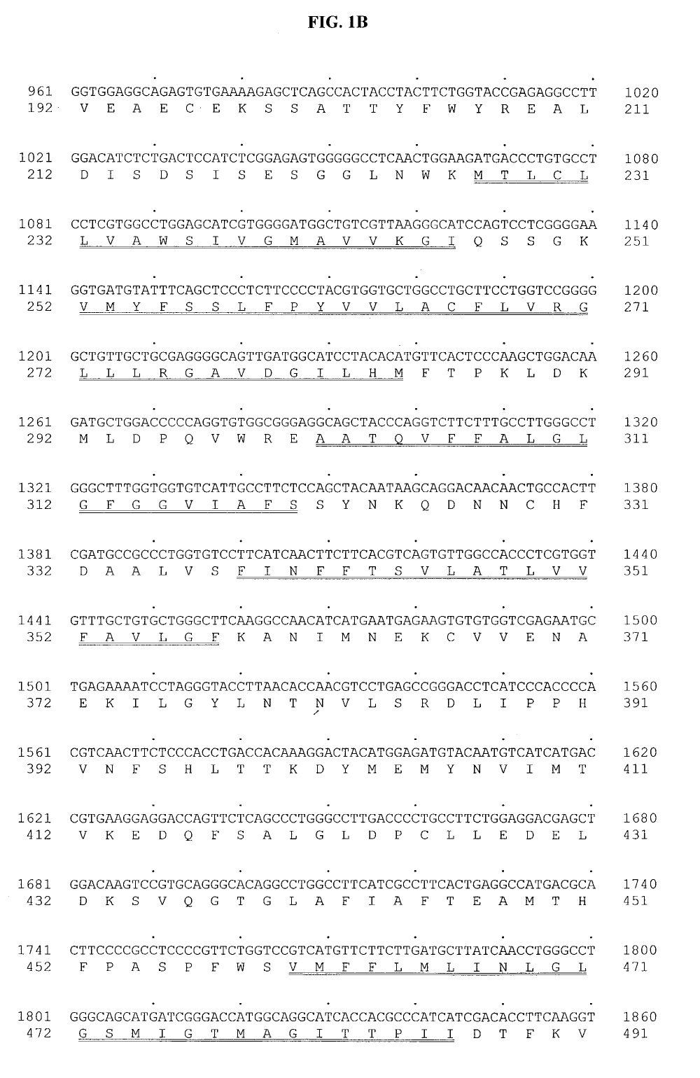

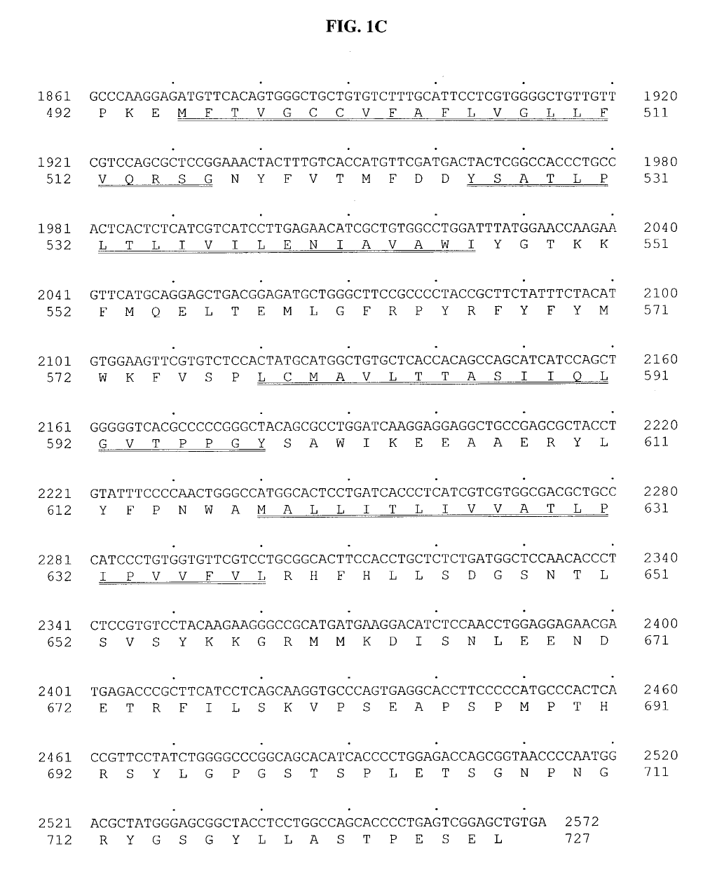

The present invention provides isolated nucleic acid molecules, that comprise, or alternatively consist of, a polynucleotide encoding the HNTTBMY1 protein having the amino acid sequence shown in FIGS. 1A-C (SEQ ID NO: 1) or the amino acid sequence encoded by the cDNA clone, HNTTBMY1 (also referred to as RS_{—}181961.1), deposited as ATCC Deposit Number PTA-4803 on Nov. 13, 2002.

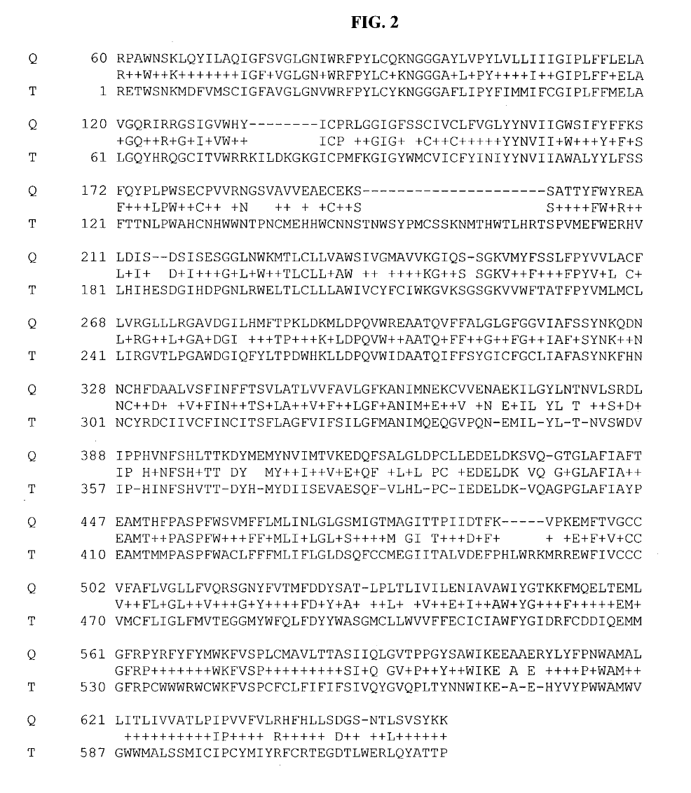

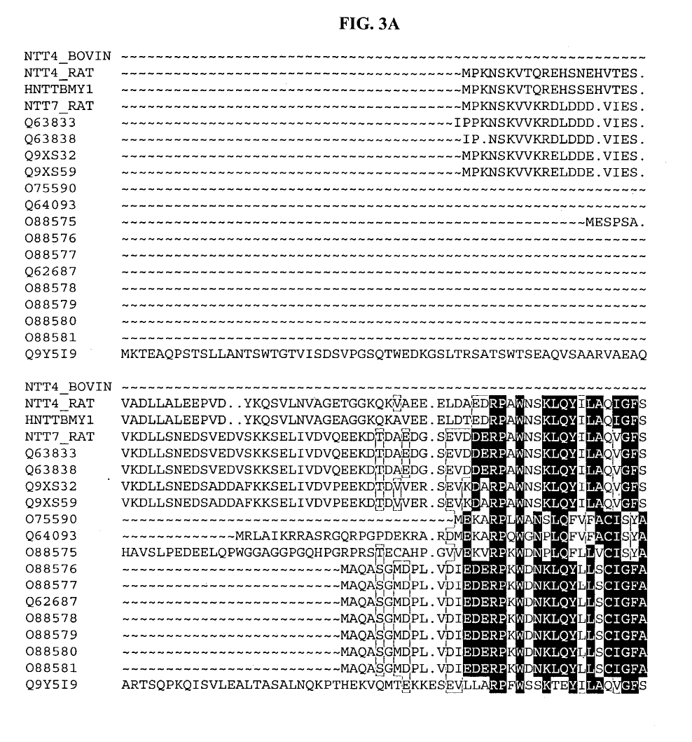

The inventive polypeptide represented by SEQ ID NO: 1 was searched against profile Hidden Markov Models of neurotransmitter transporters generated by Pfam, a database of multiple alignments of protein domains or conserved protein regions. The alignments represent certain evolutionarily conserved structures, having implications for the protein's function. A comparison of alignment of the sequence of HNTTBMY1 with the Target Pfam model is shown in FIG. 2. The results of this analysis showed that SEQ ID NO: 1 matched significantly to the transmembrane sodium symporter family Pfam model.

An algorithm was used to predict membrane spanning regions within the inventive transporter based on statistical analysis of a database of naturally occurring transmembrane proteins [Hofmann K., and Stoffel, W., Biol. Chem. Hoppe-Seyler, 347:166, (1993)]. This algorithm is the basis for the TMPRED program. A “transmembrane domain” refers to an amino acid sequence having at least about 20 to 25 amino acid residues in length and which contains at least about 65-70% hydrophobic amino acids such as alanine, leucine, phenylalanine, protein, tyrosine, tryptophan, or valine.

FIGS. 1A-C shows the transmembrane regions (double underlined) within the amino acid sequence (SEQ ID NO: 1) of the human orphan neurotransmitter transporter of the present invention as predicted using the TMPRED program. Based on this prediction, the HNTTBMY1 protein contains twelve transmembrane (TM) domains, a characteristic structural feature of Na⁺/Cl⁻ dependent neurotransmitter transporters.

The twelve HNTTBMY1 transmembrane domains (TM1 to TM12) are located from about amino acid 69 to about amino acid 90 (TM1; SEQ ID NO: 35); from about amino acid 96 to about amino acid 118 (TM2; SEQ ID NO: 36); from about amino acid 139 to about amino acid 163 (TM3; SEQ ID NO: 37); from about amino acid 227 to about amino acid 246 (TM4; SEQ ID NO: 38); from about amino acid 252 to about amino acid 284 (TM5; SEQ ID NO: 39); from about amino acid 301 to about amino acid 320 (TM6; SEQ ID NO: 40); from about amino acid 338 to about amino acid 357 (TM7; SEQ ID NO: 41); from about amino acid 460 to about amino acid 486 (TM8; SEQ ID NO: 42); from about amino acid 495 to about amino acid 516 (TM9; SEQ ID NO: 43); from about amino acid 526 to about amino acid 546 (TM10; SEQ ID NO: 44); from about amino acid 578 to about amino acid 598 (TM11; SEQ ID NO: 45); and/or from about amino acid 618 to about amino acid 638 (TM12; SEQ ID NO: 46) of SEQ ID NO: 1 (FIGS. 1A-C). In this context, the term “about” may be construed to mean 1, 2, 3, 4, 5, 6, 7, 8, 9, or 10 amino acids beyond the N-Terminus and/or C-terminus of the above referenced transmembrane domain polypeptides.

In preferred embodiments, the following transmembrane domain polypeptides are encompassed by the present invention: YILAQIGFSVGLGNIWRFPYLC (SEQ ID NO: 35), GAYLVPYLVLLIIIGIPLFFLEL (SEQ ID NO: 36), LGGIGFSSCIVCLFVGLYYNVIIGW (SEQ ID NO: 37), MTLCLLVAWSIVGMAVVKGI (SEQ ID NO: 38), VMYFSSLFPYVVLACFLVRGLLLRGAVDGILHM (SEQ ID NO: 39), AATQVFFALGLGFGGVIAFS (SEQ ID NO: 40), FINFFTSVLATLVVFAVLGF (SEQ ID NO: 41), VMFFLMLINLGLGSMIGTMAGITTPII (SEQ ID NO: 42) MFTVGCCVFAFLVGLLFVQRSG (SEQ ID NO: 43) YSATLPLTLIVILENIAVAWI (SEQ ID NO: 44), LCMAVLTTASIIQLGVTPPGY (SEQ ID NO: 45), and/or MALLITLIVVATLPIPVVFVL (SEQ ID NO: 46). Polynucleotides encoding these polypeptides are also provided. The present invention also encompasses the use of these HNTTBMY1 transmembrane domain polypeptides as immunogenic and/or antigenic epitopes as described elsewhere herein.

The present invention also encompasses the polypeptide sequences that intervene between each of the predicted HNTTBMY1 transmembrane domains. Since these regions are solvent accessible either extracellularly or intracellularly, they are particularly useful for designing antibodies specific to each region. Such antibodies may be useful as antagonists or agonists of the HNTTBMY1 full-length polypeptide and may modulate its activity.

In preferred embodiments, the following inter-transmembrane domain polypeptides are encompassed by the present invention: AVGQRIRRGSIGVWHYICPR (SEQ ID NO: 47), SIFYFFKSFQYPLPWSECPVVRNGSVAVVEAECEKSSATTYFWYREALDISDSI SESGGLNWK (SEQ ID NO: 48), FTPKLDKMLDPQVWRE (SEQ ID NO: 49), SYNKQDNNCHFDAALVS (SEQ ID NO: 50), KANIMNEKCVVENAEKILGYLNTNVLSRDLIPPHVNFSHLTTKDYMEMYNVI MTVKEDQFSALGLDPCLLEDELDKSVQGTGLAFIAFIEAMTHFPASPFWS (SEQ ID NO: 51), DTFKVPKE (SEQ ID NO: 52), NYFVTMFDD (SEQ ID NO: 53), YGTKKFMQELTEMLGFRPYRFYFYMWKFVSP (SEQ ID NO: 54), and SAWIKEEAAERYLYFPNWA (SEQ ID NO: 55).

In preferred embodiments, the following N-terminal HNTTBMY1 TM2-3 intertransmembrane domain deletion polypeptides are encompassed by the present invention: A1-R20, V2-R20, G3-R20, Q4-R20, R5-R20, I6-R20, R7-R20, R8-R20, G9-R20, S10-R20, I11-R20, G12-R20, V13-R20, and/or W14-R20 of SEQ ID NO: 47. Polynucleotide sequences encoding these polypeptides are also provided. The present invention also encompasses the use of these N-terminal HNTTBMY1 TM2-3 intertransmembrane domain deletion polypeptides as immunogenic and/or antigenic epitopes as described elsewhere herein.

In preferred embodiments, the following C-terminal HNTTBMY1 TM2-3 intertransmembrane domain deletion polypeptides are encompassed by the present invention: A1-R20, A1-P19, A1-C18, A1-I17, A1-Y16, A1-H15, A1-W14, A1-V13, A1-G12, A1-I11, A1-S10, A1-G9, A1-R8, and/or A1-R7 of SEQ ID NO: 47. Polynucleotide sequences encoding these polypeptides are also provided. The present invention also encompasses the use of these C-terminal HNTTBMY1 TM2-3 intertransmembrane domain deletion polypeptides as immunogenic and/or antigenic epitopes as described elsewhere herein.

In preferred embodiments, the following N-terminal HNTTBMY1 TM3-4 intertransmembrane domain deletion polypeptides are encompassed by the present invention: S1-K63, I2-K63, F3-K63, Y4-K63, F5-K63, F6-K63, K7-K63, S8-K63, F9-K63, Q10-K63, Y11-K63, P12-K63, L13-K63, P14-K63, W15-K63, E17-K63, C18-K63, P19-K63, V20-K63, V21-K63, R22-K63, N23-K63, G24-K63, S25-K63, V26-K63, A27-K63, V28-K63, V29-K63, E30-K63, A31-K63, E32K63, C33-K63, E34-K63, K35-K63, S36-K63, S37-K63, A38-K63, T39-K63, T40-K63, Y41-K63, F42-K63, W43-K63, Y44-K63, R45-K63, E46-K63, A47-K63, L48-K63, D49-K63, I50-K63, S51-K63, D52-K63, S53-K63, I54-K63, S55-K63, E56-K53, and/or S57-K63 of SEQ ID NO: 48. Polynucleotide sequences encoding these polypeptides are also provided. The present invention also encompasses the use of these N-terminal HNTTBMY1 TM3-4 intertransmembrane domain deletion polypeptides as immunogenic and/or antigenic epitopes as described elsewhere herein.

In preferred embodiments, the following C-terminal HNTTBMY1 TM3-4 intertransmembrane domain deletion polypeptides are encompassed by the present invention: S1-K63, S1-W62, S1-N61, S1-L60, S1-G59, S1-G58, S1-S57, S1-E56, S1-S55, S1-154, S1-S53, S1-D52, S1-S51, S1-I50, S1-D49, S1-L48, S1-A47, S1-E46, S1-R45, S1-Y44, S1-W43, S1-F42, S1-Y41, S1-T40, S1-T39, S1-A38, S1-S37, S1-S36, S1-K35, S1-E34, S1-C33, S1-E32, S1-A31, S1-E30, S1-V29, S1-V28, S1-A27, S1-V26, S1-S25, S1-G24, S1-N23, S1-R22, S1-V21, S1-V20, S1-P19, S1-C18, S1-E17, S1-S16, S1-W15, S1-P14, S1-L13, S1-P12, S1-Y11, S1-Q10, S1-F9, S1-S8, and/or S1-K7 of SEQ ID NO: 48. Polynucleotide sequences encoding these polypeptides are also provided. The present invention also encompasses the use of these C-terminal HNTTBMY1 TM3-4 intertransmembrane domain deletion polypeptides as immunogenic and/or antigenic epitopes as described elsewhere herein.

In preferred embodiments, the following N-terminal HNTTBMY1 TM5-6 intertransmembrane domain deletion polypeptides are encompassed by the present invention: F1-E16, T2-E16, P3-E16, K4-E16, L5-E16, D6-E16, K7-E16, M8-E-16, L9-E16, and/or D10-E16 of SEQ ID NO: 49. Polynucleotide sequences encoding these polypeptides are also provided. The present invention also encompasses the use of these N-terminal HNTTBMY1 TM5-6 intertransmembrane domain deletion polypeptides as immunogenic and/or antigenic epitopes as described elsewhere herein.

In preferred embodiments, the following C-terminal HNTTBMY1 TM5-6 intertransmembrane domain deletion polypeptides are encompassed by the present invention: F1-E16, F1-R15, F1-W14, F1-V13, F1-Q12, F1-P11, F1-D10, F1-L9, F1-M8, and/or F1-K7 of SEQ ID NO: 49. Polynucleotide sequences encoding these polypeptides are also provided. The present invention also encompasses the use of these C-terminal HNTTBMY1 TM5-6 intertransmembrane domain deletion polypeptides as immunogenic and/or antigenic epitopes as described elsewhere herein.

In preferred embodiments, the following N-terminal HNTTBMY1 TM6-7 intertransmembrane domain deletion polypeptides are encompassed by the present invention: S1-S17, Y2-S17, N3-S17, K4-S17, Q5-S17, D6-S17, N7-S17, N8-S17, C9-S17, H10-S17, and/or F11-S17 of SEQ ID NO: 50. Polynucleotide sequences encoding these polypeptides are also provided. The present invention also encompasses the use of these N-terminal HNTTBMY1 TM6-7 intertransmembrane domain deletion polypeptides as immunogenic and/or antigenic epitopes as described elsewhere herein.

In preferred embodiments, the following C-terminal HNTTBMY1 TM6-7 intertransmembrane domain deletion polypeptides are encompassed by the present invention: S1-S17, S1-V16, S1-L15, S1-A14, S1-A13, S1-D12, S1-F11, S1-H10, S1-C9, S1-N8, and/or S1-N7 of SEQ ID NO: 50. Polynucleotide sequences encoding these polypeptides are also provided. The present invention also encompasses the use of these C-terminal HNTTBMY1 TM6-7 intertransmembrane domain deletion polypeptides as immunogenic and/or antigenic epitopes as described elsewhere herein.

In preferred embodiments, the following N-terminal HNTTBMY1 TM7-8 intertransmembrane domain deletion polypeptides are encompassed by the present invention: K1-S102, A2-S102, N3-S102, I4-S102, M5-S102, N6-S102, E7-S102, K8-S102, C9-S102, V10-S102, V11-S102, E12-S102, N13-S102, A14-S102, E15-S102, K16-S102, I17-S102, L18-S102, G19-S102, Y20-S102, L21-S102, N22-S102, T23-S102, N24-S102, V25-S102, L26-S102, S27-S102, R28-S102, D29-S102, L30-S102, I31-S102, P32-S102, P33-S102, H34-S102, V35-S102, N36-S102, F37-S102, S38-S102, H39-S102, L40-S102, T41-S102, T42-S102, K43-S102, D44-S102, Y45-S102, M46-S102, E47-S102, M48-S102, Y49-S102, N50-S102, V51-S102, I52-S102, M53-S102, T54-S102, V55-S102, K56-S102, E57-S102, D58-S102, Q59-S102, F60-S102, S61-S102, A62-S102, L63-S102, G64-S102, L65-S102, D66-S102, P67-S102, C68-S102, L69-S102, L70-S102, E71-S102, D72-S102, E73-S102, L74-S102, D75-S102, K76-S102, S77-S102, V78-S102, Q79-S102, G80-S102, T81-S102, G82-S102, L83-S102, A84-S102, F85-S102, I86-S102, A87-S102, F88-S102, T89-S102, E90-S102, A91-S102, M92-S102, T93-S102, H94-S102, F95-S102, and/or P96-S102 of SEQ ID NO: 51. Polynucleotide sequences encoding these polypeptides are also provided. The present invention also encompasses the use of these N-terminal HNTTBMY1 TM7-8 intertransmembrane domain deletion polypeptides as immunogenic and/or antigenic epitopes as described elsewhere herein.

In preferred embodiments, the following C-terminal HNTTBMY1 TM7-8 intertransmembrane domain deletion polypeptides are encompassed by the present invention: K1-S102, K1-W101, K1-F100, K1-P99, K1-S98, K1-A97, K1-P96, K1-F95, K1-H94, K1-T93, K1-M92, K1-A91, K1-E90, K1-T89, K1-F88, K1-A87, K1-I86, K1-F85, K1-A84, K1-L83, K1-G82, K1-T81, K1-G80, K1-Q79, K1-V78, K1-S77, K1-K76, K1-D75, K1-L74, K1-E73, K1-D72, K1-E71, K1-L70, K1-L69, K1-C68, K1-P67, K1-D66, K1-L65, K1-G64, K1-L63, K1-A62, K1-S61, K1-F60, K1-Q59, K1-D58, K1-E57, K1-K56, K1-V55, K1-T54, K1-M53, K1-I52, K1-V51, K1-N50, K1-Y49, K1-M48, K1-E47, K1-M46, K1-Y45, K1-D44, K1-K43, K1-T42, K-1-T41, K1-L40, K1-H39, K1-S38, K1-F37, K1-N36, K1-V35, K1-H34, K1-P33, K1-P32, K1-I31, K1-L30, K1-D29, K1-R28, K1-S27, K1-L26, K1-V25, K1-N24, K1-T23, K1-N22, K1-L21, K1-Y20, K1-G19, K1-L18, K1-I17, K1-K16, K1-E15, K1-A14, K1-N13, K1-E12, K1-V11, K1-V10, K1-C9, K1-K8, and/or K1-E7 of SEQ ID NO: 51. Polynucleotide sequences encoding these polypeptides are also provided. The present invention also encompasses the use of these C-terminal HNTTBMY1 TM7-8 intertransmembrane domain deletion polypeptides as immunogenic and/or antigenic epitopes as described elsewhere herein.

In preferred embodiments, the following N-terminal HNTTBMY1 TM8-9 intertransmembrane domain deletion polypeptides are encompassed by the present invention: D1-E8, and/or T2-E8 of SEQ ID NO: 52. Polynucleotide sequences encoding these polypeptides are also provided. The present invention also encompasses the use of these N-terminal HNTTBMY1 TM8-9 intertransmembrane domain deletion polypeptides as immunogenic and/or antigenic epitopes as described elsewhere herein.

In preferred embodiments, the following C-terminal HNTTBMY1 TM8-9 intertransmembrane domain deletion polypeptides are encompassed by the present invention: D1-E8, and/or D1-K7 of SEQ ID NO: 52. Polynucleotide sequences encoding these polypeptides are also provided. The present invention also encompasses the use of these C-terminal HNTTBMY1 TM8-9 intertransmembrane domain deletion polypeptides as immunogenic and/or antigenic epitopes as described elsewhere herein.

In preferred embodiments, the following N-terminal HNTTBMY1 TM9-10 intertransmembrane domain deletion polypeptides are encompassed by the present invention: N1-D9, Y2-D9, and/or F3-D9 of SEQ ID NO: 53. Polynucleotide sequences encoding these polypeptides are also provided. The present invention also encompasses the use of these N-terminal HNTTBMY1 TM9-10 intertransmembrane domain deletion polypeptides as immunogenic and/or antigenic epitopes as described elsewhere herein.

In preferred embodiments, the following C-terminal HNTTBMY1 TM9-10 intertransmembrane domain deletion polypeptides are encompassed by the present invention: N1-D9, N1-D8, and/or N1-F7 of SEQ ID NO: 53. Polynucleotide sequences encoding these polypeptides are also provided. The present invention also encompasses the use of these C-terminal HNTTBMY1 TM9-10 intertransmembrane domain deletion polypeptides as immunogenic and/or antigenic epitopes as described elsewhere herein.

In preferred embodiments, the following N-terminal HNTTBMY1 TM10-11 intertransmembrane domain deletion polypeptides are encompassed by the present invention: Y1-P31, G2-P31, T3-P31, K4-P31, K5-P31, F6-P31, M7-P31, Q8-P31, E9-P31, L10-P31, T11-P31, E12-P31, M13-P31, L14-P31, G15-P31, F16-P31, R17-P31, P18-P31, Y19-P31, R20-P31, F21-P31, Y22-P31, F23-P31, Y24-P31, and/or M25-P31 of SEQ ID NO: 54. Polynucleotide sequences encoding these polypeptides are also provided. The present invention also encompasses the use of these N-terminal HNTTBMY1 TM10-11 intertransmembrane domain deletion polypeptides as immunogenic and/or antigenic epitopes as described elsewhere herein.

In preferred embodiments, the following C-terminal HNTTBMY1 TM10-11 intertransmembrane domain deletion polypeptides are encompassed by the present invention: Y1-P31, Y1-S30, Y1-V29, Y1-F28, Y1-K27, Y1-W26, Y1-M25, Y1-Y24, Y1-F23, Y1-Y22, Y1-F21, Y1-R20, Y1-Y19, Y1-P18, Y1-R17, Y1-F16, Y1-G15, Y1-L14, Y1-M13, Y1-E12, Y1-T11, Y1-L10, Y1-E9, Y1-Q8, and/or Y1M-7 of SEQ ID NO: 54. Polynucleotide sequences encoding these polypeptides are also provided. The present invention also encompasses the use of these C-terminal HNTTBMY1 TM10-11 intertransmembrane domain deletion polypeptides as immunogenic and/or antigenic epitopes as described elsewhere herein.

In preferred embodiments, the following N-terminal HNTTBMY1 TM11-12 intertransmembrane domain deletion polypeptides are encompassed by the present invention: S1-A19, A2-A19, W3-A19, I4-A19, K5-A19, E6-A19, E7-A19, A8A-19, A9-A19, E10-A19, R11-A19, Y12-A19, and/or L13-A19 of SEQ ID NO: 55. Polynucleotide sequences encoding these polypeptides are also provided. The present invention also encompasses the use of these N-terminal HNTTBMY1 TM11-12 intertransmembrane domain deletion polypeptides as immunogenic and/or antigenic epitopes as described elsewhere herein.

In preferred embodiments, the following C-terminal HNTTBMY1 TM11-12 intertransmembrane domain deletion polypeptides are encompassed by the present invention: S1-A19, S1-W18, S1-N17, S1-P16, S1-F15, S1-Y14, S1-L13, S1-Y12, S1-R11, S1-E10, S1-A9, S1-A8, and/or S1-E7 of SEQ ID NO: 55. Polynucleotide sequences encoding these polypeptides are also provided. The present invention also encompasses the use of these C-terminal HNTTBMY1 TM11-12 intertransmembrane domain deletion polypeptides as immunogenic and/or antigenic epitopes as described elsewhere herein.

In preferred embodiments, the present invention encompasses the use of N-terminal deletions, C-terminal deletions, or any combination of N-terminal and C-terminal deletions of any one or more of the HNTTBMY1 TM1 thru TM12 transmembrane domain polypeptides as antigenic and/or immunogenic epitopes.

In preferred embodiments, the present invention also encompasses the use of N-terminal deletions, C-terminal deletions, or any combination of N-terminal and C-terminal deletions of any one or more of the amino acids intervening (i.e., GPCR extracellular or intracellular loops) the HNTTBMY1 TM1 thru TM12 transmembrane domain polypeptides as antigenic and/or immunogenic epitopes.

Referring now to FIG. 4, a hydrophilicity plot of the human neurotransmitter transporter of the invention is shown. Relatively hydrophilic regions are above the horizontal line, and relatively hydrophobic regions are below the horizontal line. The hydrophobic regions indicate the 12 TM domains in the protein. It should be noted that relatively hydrophilic regions are generally located at or near the surface of a protein, and are more frequently effective immunogenic epitopes than are relatively hydrophobic regions.

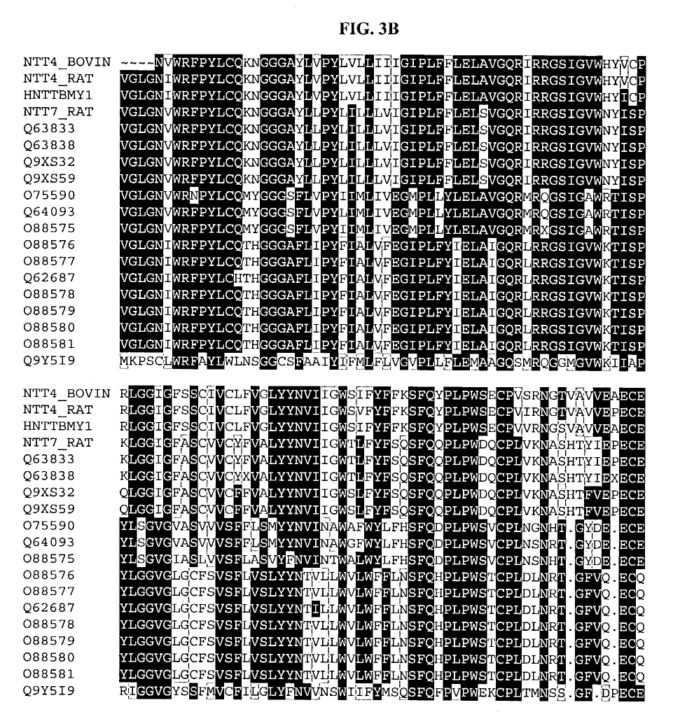

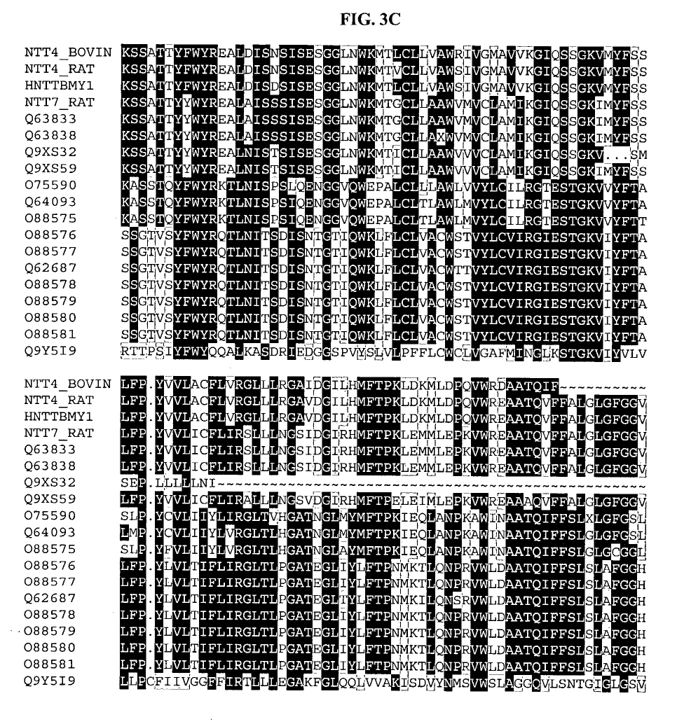

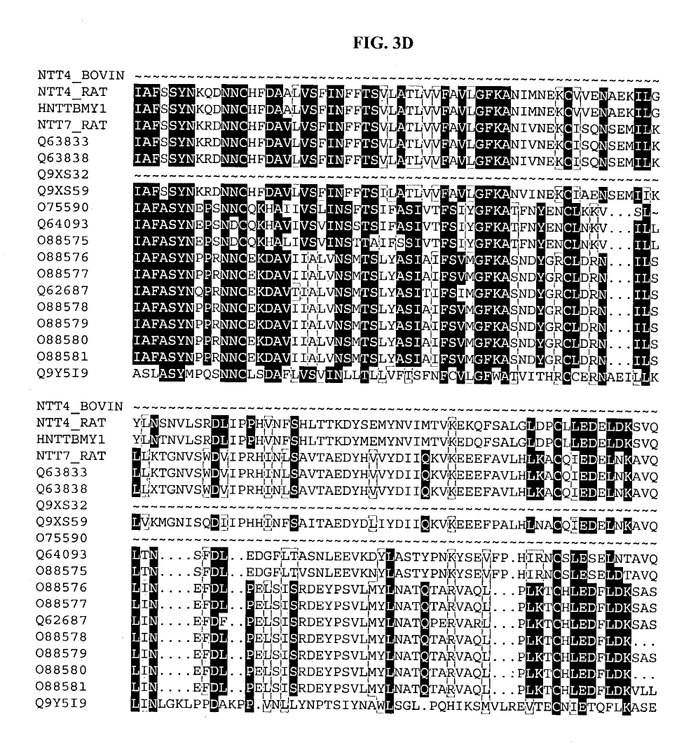

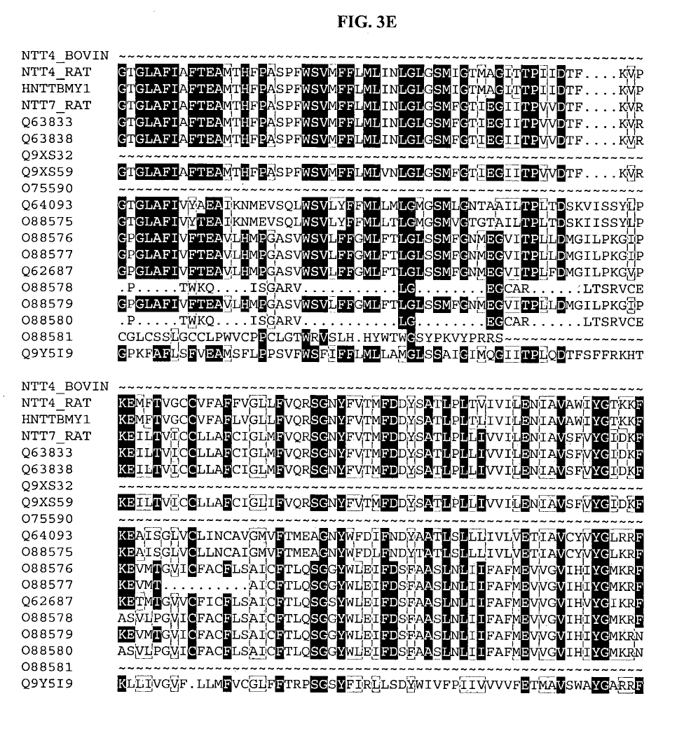

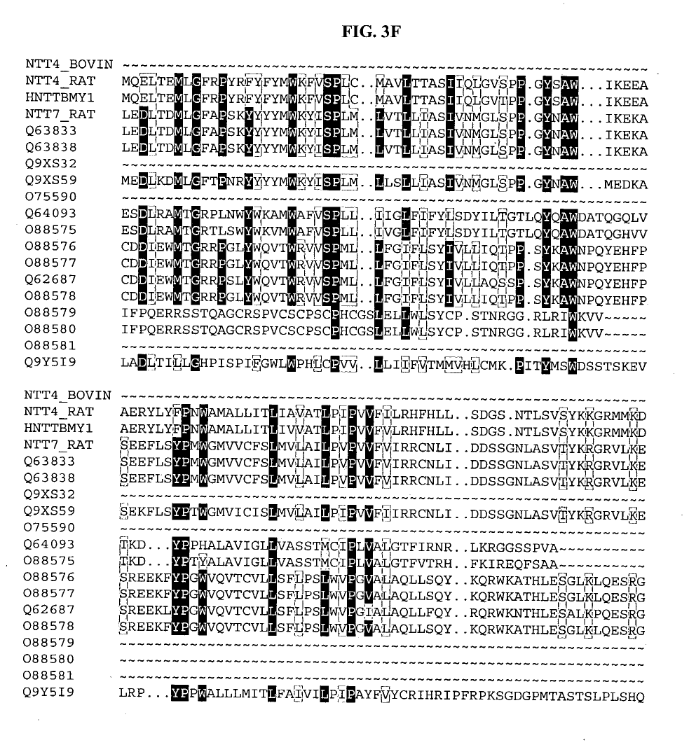

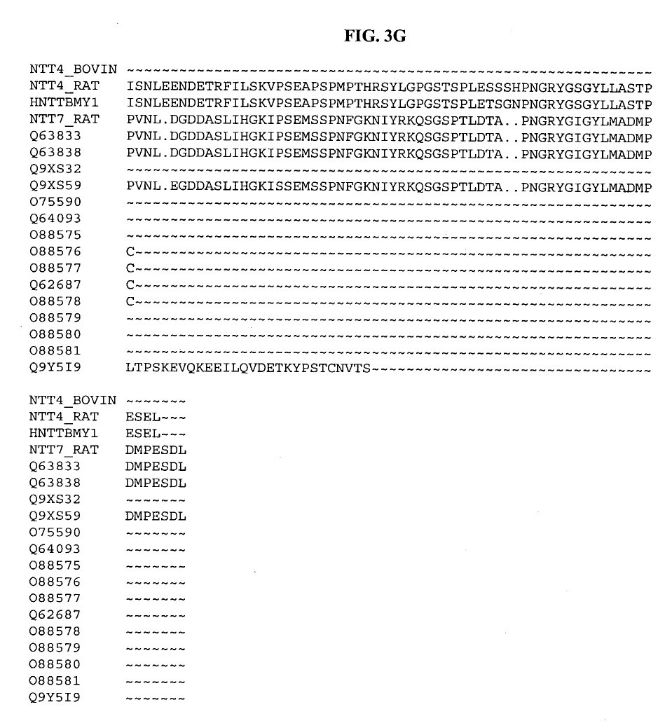

The amino acid sequence (SEQ ID NO: 1) of the orphan neurotransmitter of the present invention was aligned with other orphan neurotransmitter transporter sequences (SEQ ID NOs: 12-29) as shown in FIG. 3 and in Table I below. Referring now to Table I, the inventive human neurotransmitter transporter shares approximately 96.8% sequence identity with a Na⁺/Cl⁻ dependent orphan neurotransmitter transporter from rat termed NTT4 (also referred to as Rxt1 and rat xt1). Moreover, the inventive transporter shows approximately 96.0% identity with bovine NTT4. As a result of this high degree of homology to the rat and bovine NTT4 transporters, it was concluded that the inventive cDNA sequence of SEQ ID NO: 2 resulted from an identical gene of the human genome. Furthermore, the inventive transporter shows between about 37 to about 70% identity with other orphan transporters listed in Table I. The percent identity and percent similarity values were determined using the Gap algorithm using default parameters (Genetics Computer Group suite of programs; Needleman and Wunsch. J. Mol. Biol. 48; 443-453, 1970); GAP parameters: gap creation penalty: 8 and gap extension penalty: 2).

TABLE I
Protein Sequence Comparison of Inventive
Human Neurotransmitter Transporter With Other
Orphan Transporters Used in the Alignment of FIGS. 3A-G

| | | | | % |
|---|---|---|---|---|
| | | | | Sequence |
| | | | | Identity/ |
| | SWISS- | | | Simlarity |
| | PROT | | | With |
| SEQ ID | Accession | | | SEQ ID |
| NO. | No. | Name | Species | NO:1 |
| 12 | Q28001 | NTT4_BOVIN | Bos taurus | 96.0/98.2 |
| 13 | P31662 | NTT4_RAT | Rattus norvegicus | 96.8/97.4 |
| 14 | Q08469 | NTT7_RAT | Rattus norvegicus | 67.2/76.5 |
| 15 | 075590 | Orphan Transporter | Homo sapiens | 51.1/61.6 |
| | | (Fragment) | | |
| 16 | 088575 | Orphan Transporter | Mus musculus | 44.4/54.3 |
| 17 | 088576 | Isoform A12 | Mus musculus | 43.8/56.0 |
| 18 | 088577 | Isoform A11 | Mus musculus | 43.4/55.7 |
| 19 | 988578 | Isoform B11 | Mus musculus | 41.7/54.3 |
| 20 | 088579 | Isoform A10 | Mus musculus | 45.4/56.4 |
| 21 | 088580 | Isoform B9 | Mus musculus | 43.1/54.5 |
| 22 | 088681 | Isoform A8 | Mus musculus | 44.8/56.2 |
| 23 | Q62687 | Renal Osmotic | Rattis norvegicus | 42.9/54.6 |
| | | Stress-Induced | | |
| | | Na⁺/Cl Organic | | |
| | | Solute Cotransporter | | |
| 24 | Q63833 | Sodium Dependent | Rattus norvegicus | 67.0/76.3 |
| | | Neurotransmitter | | |
| | | Transporter | | |
| 25 | Q63838 | Sodium Dependent | Rattus norvegicus | 66.7/76.0 |
| | | Neurotransmitter | | |
| | | Transporter | | |
| 26 | Q64093 | Neurotransmitter | Rattus norvegicus | 45.1/55.1 |
| | | Transporter RB21A | | |
| 27 | Q9XS32 | Orphan Transporter | Bos taurus | 69.5/76.7 |
| | | Short Splicing | | |
| | | Variant | | |
| 28 | Q9XC59 | Orphan Transporter | Bos taurus | 67.0/76.5 |
| | | Short Splicing | | |
| | | Variant | | |
| 29 | Q9Y519 | NTT5 | Homo sapiens | 36.7/48.1 |

Additional information relative to the HNTTBMY1 homologues provided above may be found be reference to the following publications: FEBS Lett. 315:114-118(1993); J. Neurochem. 62:445-455(1994); Brain Res. Mol. Brain Res. 16:353-359(1992); Recept. Channels 6:113-128(1998); Am. J. Physiol. 267:F688-F694(1994); Brain Res. Mol. Brain Res. 16:353-359(1992); FEBS Lett. 357:86-92(1995); and Soc. Neurosci. 24:1606-1606(1998); which are hereby incorporated herein by reference in their entirety.

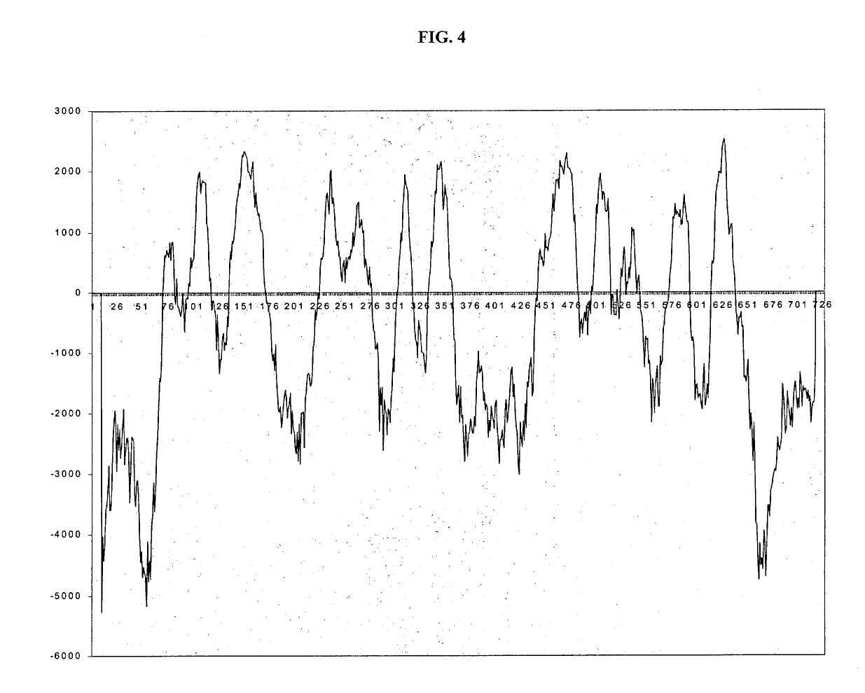

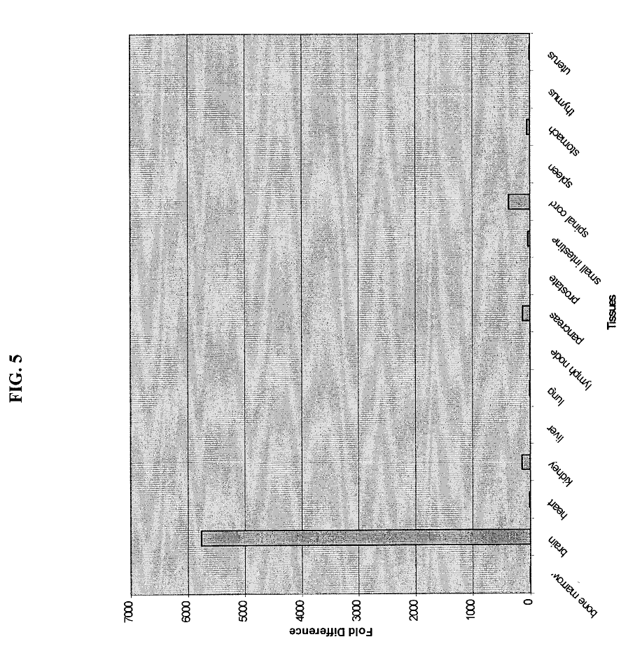

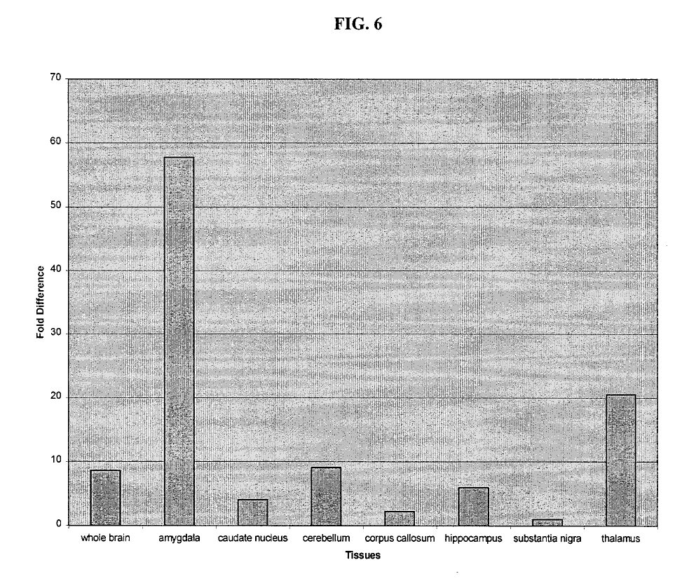

RT-PCR expression profiling experiments indicated that mRNA corresponding to the cDNA encoding HNTTBMY1 is expressed most highly in the brain and is expressed to a much lesser extent in the spinal cord, kidney, and pancreas. The expression profile for various organs is shown in FIG. 5. The expression profile performed on brain subregions, shown in FIG. 6, reveals that HNTTBMY1 is highly expressed in the amygdla, and to a lesser degree in thalamus, cerebellum, hippocampus and caudate nucleus. Its presence in brain suggests that its endogenous substrate may be neuroactive. Its cloning provides the means to determine its functions in the nervous system.

There is growing evidence that diseases related to long term behavioral changes are heritable in 30% or more of the identified cases. Availability of the human genome sequences and identification of single nucleotide polymorphisms (SNP's) have been making it possible to determine the genetic variation (heterozygosity) for heritable diseases in the general population. Analysis of heterozygosity in the NTT genes, in particular orphan NTT genes, and its association with various diseases and disease susceptibilities, enable new methods of diagnosis, prevention and treatment of a variety of these disorders to be developed. In particular, the inventive transporter will likely be useful for the development of therapeutic agents for neurological and psychiatric disorders.

In the present invention, the full length sequence identified as SEQ ID NO: 2 was often generated by overlapping sequences contained in one or more clones (contig analysis). A representative clone containing all or most of the sequence for SEQ ID NO: 2 was deposited with the American Type Culture Collection (“ATCC”). The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure. The deposited clone is inserted in the pSport plasmid (Life Technologies) using the Not I and Sal I restriction endonucleases as described herein.

In preferred embodiments, the encoding polynucleotide sequence of the HNTTBMY1 polypeptide are represented by nucleotides 380 to 2569 of SEQ ID NO: 2.

Polypeptides of the present invention, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptides may also be modified with a detectable label, such as an enzymatic, fluorescent, isotopic or affinity label to allow for detection and isolation of the protein, the addition of epitope tagged peptide fragments (e.g., FLAG, HA, GST, thioredoxin, maltose binding protein, etc.), attachment of affinity tags such as biotin and/or streptavidin, the covalent attachment of chemical moieties to the amino acid backbone, N- or C-terminal processing of the polypeptides ends (e.g., proteolytic processing), deletion of the N-terminal methionine residue, etc.

Therefore, the present invention is also directed to a polynucleotide encoding the HNTTBMY1 polypeptide that lacks the start methionine. Specifically, the present invention encompasses nucleotides 383 to 2569 of SEQ ID NO: 2. The invention also encompasses amino acids 2 to 727 of SEQ ID NO: 1.

The molecular weight of the HNTTBMY1 polypeptide shown in FIGS. 1A-C (SEQ ID NO: 1) was predicted to be about 81 kD.

It is another aspect of the present invention to provide modulators of the HNTTBMY1 protein and HNTTBMY1 peptide targets which can affect the function or activity of HNTTBMY1 in a cell in which HNTTBMY1 function or activity is to be modulated or affected. In addition, modulators of HNTTBMY1 can affect downstream systems and molecules that are regulated by, or which interact with, HNTTBMY1 in the cell. Modulators of HNTTBMY1 include compounds, materials, agents, drugs, and the like, that antagonize, inhibit, reduce, block, suppress, diminish, decrease, or eliminate HNTTBMY1 function and/or activity. Such compounds, materials, agents, drugs and the like can be collectively termed “antagonists”. Alternatively, modulators of HNTTBMY1 include compounds, materials, agents, drugs, and the like, that agonize, enhance, increase, augment, or amplify HNTTBMY1 function in a cell. Such compounds, materials, agents, drugs and the like can be collectively termed “agonists”.

In one embodiment, a HNTTBMY1 polypeptide comprises a portion of the amino sequence depicted in FIGS. 1A-C. In another embodiment, a HNTTBMY1 polypeptide comprises at least 4, 5, 6, 7, 8, 9, 10, 11, 12, 13, 14, 15, 16, 17, 18, 19 or 20 amino acids of the amino sequence depicted in FIGS. 1A-C. In further embodiments, the following HNTTBMY1 polypeptide fragments are specifically excluded from the present invention: VWREAATQVFFALGLGFGGVIAFSSYNK (SEQ ID NO: 99); LVSFINFFTSVLATLVVFAVLGFKANI (SEQ ID NO: 100); EEELDTEDRPAWNSKLQYILAQIGFSVGLGNIWRFPYLCQKNGGGAYLVPYL VLLIIIGIPLFFLELAVGQRIRRGSIGVWHY (SEQ ID NO: 101); CPRLGGIGFSSCIVCLFVGLYYNVIIGWS (SEQ ID NO: 102); FYFFKSFQYPLPWSECPV (SEQ ID NO: 103); ECEKSSATTYFWYREALDIS (SEQ ID NO: 104), SISESGGLNWKMTLC(SEQ ID NO: 105); IVGMAVVKGIQSSGKV(SEQ ID NO: 106); VQGTGLAFIAFTEAMTHFPASPFWSVMFFLMLINLGLGSM(SEQ ID NO: 107); VCLFVGLYYNVIIGWS(SEQ ID NO: 108); FYFFKSFQYPLPWSECPV(SEQ ID NO: 109); ECEKSSATTYFWYREALDIS(SEQ ID NO: 110); SISESGGLNWKMTLCLLVAWSIVGMAVVKGIQSSGKVMYFSSLFPYVVLACF LVRGLLLRGAVDGILHMFTPKLDKMLDPQVWREAATQVFFALGLGFGGVIA FSSY(SEQ ID NO: 111); KQDNNCHFDAALVSFINFFTSVLATLVVFAVLGFKANIMNEKCVVENAEK(SE Q ID NO: 112); DNNCHFDALVSFINFFTSVLATLVVFAVLGFKAN(SEQ ID NO: 113); VQGTGLAFIAFTEAMTHFPASPFWSVMFFLML(SEQ ID NO: 114); AVAWIYGTKKFMQELTEMLGF(SEQ ID NO: 115); PYRFYFYMWKFVSPLCMAVLTTASIIQLGV(SEQ ID NO: 116); PPGYSAWIKEEAAERYLYFP(SEQ ID NO: 117); LRHFHLLSDGSNTLSVSYKKGRMMKDISNLEENDETRFILSKVPSEAPSPMPT HRSYLGPGSTSPL(SEQ ID NO: 118); NPNGRYGSGYLLASTPESEL(SEQ ID NO: 119); PYRFYFYMWKFVSPLCMAVLTTASIIQLGV(SEQ ID NO: 120); PPGYSAWIKEEAAERYLYFPNWAMALLITLI(SEQ ID NO: 121); LRHFHLLSDGSNTLSVSYKKGRMMKDISNLEEND(SEQ ID NO: 122); TRFILSKVPSEAPSPMPTHRSYLGPGSTSPL(SEQ ID NO: 123); RPAWNSKLQYILAQ(SEQ ID NO: 124); WRFPYLCQKNGGGAYL(SEQ ID NO: 125); LAFIAFTEAMTHFPASPFWSVMFFLMLINLGLGSM(SEQ ID NO: 126); FVQRSGNYFVTMFDDYSATLPL(SEQ ID NO: 127); TGLAFIAFTEAMTHFPASPFWSVMF(SEQ ID NO: 128); EHVTESVADLLALEEPVDYKQSVLNVAGE(SEQ ID NO: 129); EEELDTEDRPAWNSKLQYILAQIGFSVGLGNIWRFPYLCQKNGGGAYLVPYL VLLIIIGIPLFFLELAVGQRIRRGSIGVWHY(SEQ ID NO: 130); LGGIGFSSCIVCLFVGLYYNVIIGWS(SEQ ID NO: 131); FYFFKSFQYPLPWSECPV(SEQ ID NO: 132); PYRFYFYMWKFVSPLCMAVLTTASIIQLGVTPPGYSAWI(SEQ ID NO: 133); LPIPVVFVLRHFHLLSDGSNTLSVSYKKGRMMKDISNLEENDETRFELSKVPSE APSPMPTHRSYLGPGSTSPLETSGNPNGRYGSGYLLA(SEQ ID NO: 134); EEELDTEDRPAWNSKLQYILAQIGFSVGLGNfWRFPYLCQKNGGGAYLVPYL VLLIIIGIPLFFLELAVG(SEQ ID NO: 135); and/or IVGMAVVKGIQSSGKVMYFSSLFPYVVLACFLVRGLLLRGAVDGILHMFTPK LDKMLDPQVWREAATQVFFALGLGFGGVIAFSSYNKQDNNCHFDAALVSFIN FFTSVLATLVVFAVLGFKANIMNE(SEQ ID NO: 136).

TABLE II

| | | ATCC | | | | 5' NT of | | | Total |
|---|---|---|---|---|---|---|---|---|---|
| | | Deposit | | NT SEQ | Total NT | Start | | AA Seq | AA |
| Gene | CDNA | No. Z and | | ID. No. | Seq of | Codon of | 3' NT | ID No. | of |
| No. | CloneID | Date | Vector | X, | Clone | ORF | of ORF | Y | ORF |
| 1. | HNTI | PTA-4803 | pSport1 | 2 | 380 | 1 | 2569 | 1 | 727 |
| | MY1 | Nov. 13, 2002 | | | | | | | |
| | (also | | | | | | | | |
| | referred | | | | | | | | |
| | to as | | | | | | | | |
| | RS_1819 | | | | | | | | |
| | 61.1) | | | | | | | | |

Table II summarizes the information corresponding to each “Gene No.” described above. The nucleotide sequence identified as “NT SEQ ID NO: X” was assembled from partially homologous (“overlapping”) sequences obtained from the “cDNA clone ID” identified in Table II and, in some cases, from additional related DNA clones. The overlapping sequences were assembled into a single contiguous sequence of high redundancy (usually several overlapping sequences at each nucleotide position), resulting in a final sequence identified as SEQ ID NO: X. However, for the purposes of the present invention, SEQ ID NO: X may refer to any polynucleotide of the present invention.

The cDNA Clone ID was deposited on the date and given the corresponding deposit number listed in “ATCC Deposit No:Z and Date.” “Vector” refers to the type of vector contained in the cDNA Clone ID.

“Total NT Seq. Of Clone” refers to the total number of nucleotides in the clone contig identified by “Gene No.” The deposited clone may contain all or most of the sequence of SEQ ID NO: X. The nucleotide position of SEQ ID NO: X of the putative start codon (methionine) is identified as “5′ NT of Start Codon of ORF.”

The translated amino acid sequence, beginning with the methionine, is identified as “AA SEQ ID NO: Y” although other reading frames can also be easily translated using known molecular biology techniques. The polypeptides produced by these alternative open reading frames are specifically contemplated by the present invention.

The total number of amino acids within the open reading frame of SEQ ID NO: Y is identified as “Total AA of ORF”.

SEQ ID NO: X (where X may be any of the polynucleotide sequences disclosed in the sequence listing) and the translated SEQ ID NO: Y (where Y may be any of the polypeptide sequences disclosed in the sequence listing) are sufficiently accurate and otherwise suitable for a variety of uses well known in the art and described further herein. For instance, SEQ ID NO: X is useful for designing nucleic acid hybridization probes that will detect nucleic acid sequences contained in SEQ ID NO: X or the cDNA contained in the deposited clone. These probes will also hybridize to nucleic acid molecules in biological samples, thereby enabling a variety of forensic and diagnostic methods of the invention. Similarly, polypeptides identified from SEQ ID NO: Y may be used, for example, to generate antibodies which bind specifically to proteins containing the polypeptides and the proteins encoded by the cDNA clones identified in Table II.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides may cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO: 1 and the predicted translated amino acid sequence identified as SEQ ID NO: 2, but also a sample of plasmid DNA containing a cDNA of the invention deposited with the ATCC, as set forth in Table II. The nucleotide sequence of each deposited clone can readily be determined by sequencing the deposited clone in accordance with known methods. The predicted amino acid sequence can then be verified from such deposits. Moreover, the amino acid sequence of the protein encoded by a particular clone can also be directly determined by peptide sequencing or by expressing the protein in a suitable host cell containing the deposited cDNA, collecting the protein, and determining its sequence.

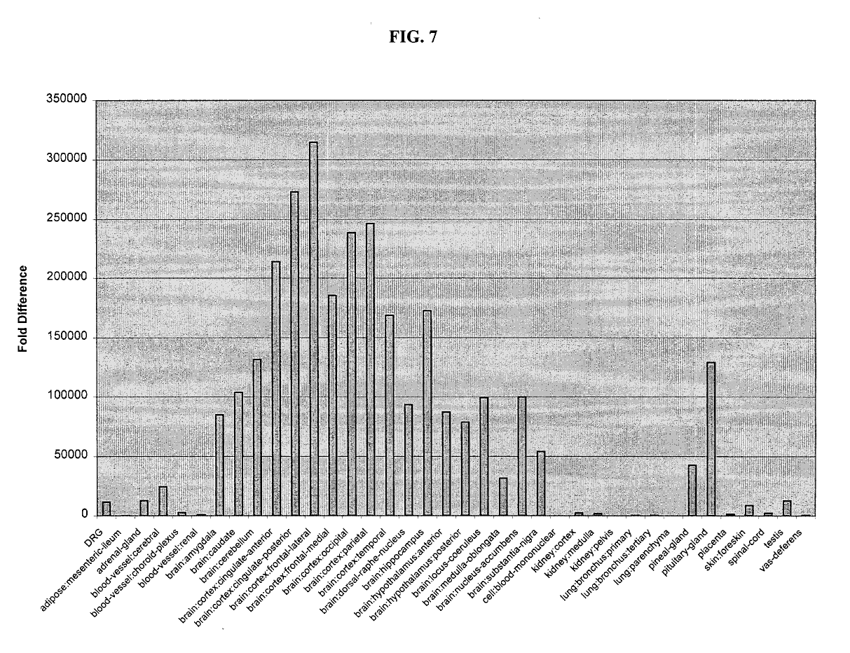

Expanded analysis of HNTTBMY1 expression levels by TaqMan (quantitative PCR (see FIGS. 5 and 6) confirmed that the HNTTBMY1 polypeptide is expressed primarily in tissues of the central nervous system (FIG. 7). HNTTBMY1 mRNA was expressed predominately in the cortex. Expression of HNTTBMY1 was also significantly expressed in the hippocampus, cerebellum, the pituitary, the locus coeruleus, the dorsal raphe nucleus, the nucleus accumbens, the substantia nigra, the pineal gland, the hypothalamus, the caudate, the amygdala, and to a lesser extent in other regions as shown. Transcripts for HNTTBMY1 are also found in low relative abundance in the spinal cord and DRG.

Collectively the expression data suggests a role for HNTTBMY in a diverse set of neural processes, including executive functions concerned with the organization of behavior, memory and cognitive function. HNTTBMY1 expression in the dorsal raphe, the site of origin of the serotonin nervous system, suggests that this neurotransmitter transporter could participate in the control of anxiety, fear, depression, sleep and pain. Expression in the locus coeruleus suggests involvement in the maintenance of an attentive or alert state. Expression in the nucleus accumbens, the region of the brain best known as the ‘reward center’ effecting the release of neurotransmnitters such as dopamine, opioid peptides, serotonin, GABA, and glutamate suggests a possible role in the establishment of addictive behaviors. Expression in the hypothalamus suggest a possible involvement the control of a diverse set of homeostatic and neuroendocrine functions, while expression in the hippocampus suggest a role in the establishment of long term potentiation. Expression in the pineal gland suggests a possible involvement in the establishment and maintenance of circadian rhythms and the control of the sleep/wake cycle. Expression in the substantia nigra suggests a possible involvement with the dopaminergic functions that emanate from this region. Expression in the DRG and the spinal cord suggest roles in various neuronal transmission systems, most notably pain.

### Isolated or Cloned Nucleic Acid Molecules

The present invention provides an isolated or recombinant polynucleotide encoding the human neurotransmitter transporter represented by the nucleotide sequence of SEQ ID NO: 2. By the term “isolated” it is meant that the material is removed from its original environment (e.g. the natural environment, if it is naturally occurring). For example, a naturally-occurring polynucleotide (or polypeptide) present in a living animal is not isolated, but the same polynucleotide or polypeptide, separated from some or all of the co-existing materials in the natural system, is isolated. The inventive transporter is referred to as HNTTBMY1 polypeptide. The invention further provides nucleic acid forms which encode a fragment of the HNTTBMY1 polypeptide. Also included within the scope of the present invention are altered (mutant) nucleic acid sequences encoding the HNTTBMY1 polypeptide or a fragment thereof. Such alterations may include, but are not limited to, deletions, insertions, or substitutions of different nucleotides. In one embodiment, these alterations result in a nucleic acid form that encodes the same or a functionally equivalent HNTTBMY1 protein. In a further embodiment, a mutant nucleic acid form may mediate or modulate a transmitter transporter activity associated with the inventive polypeptide of SEQ ID NO: 1. As defined herein, a mutation includes alterations, derivatives and variants. The variant of the polynucleotide may be a naturally occurring allelic variant of the polynucleotide or a non-naturally occurring variant of the polynucleotide.

The encoded protein may also contain deletions, insertions, or substitutions of amino acid residues. In one embodiment, such alterations may produce a silent change that results in a functionally equivalent HNTTBMY1 protein. Altered nucleic acid sequences and their encoded proteins may be useful for therapeutic purposes. This aspect of the present invention is further described below.

In one embodiment of the invention, a polynucleotide of the invention includes a 5′ and/or 3′ regulatory sequence. The regulatory sequence may be an expression control sequence. The 5′ and/or 3′ regulatory sequence may be operably linked to the nucleotide sequence encoding the transporter. The term “operably linked” in this context means that the nucleotide sequence of interest is linked to the regulatory sequence(s) in a manner which allows for expression of the nucleotide sequence (i.e., in an in vitro transcription/translation system or in a host cell when the vector is introduced into the host cell). The term “expression control sequence” is intended to include promoters, enhancers and other expression control elements.

An isolated or recombinant 5′ untranslated nucleotide sequence upstream of a nucleotide sequence encoding the human transporter of the invention is also within the purview of the invention, the 5′ untranslated sequence including the nucleotide sequence of SEQ ID NO: 3 or a fragment or a mutant form thereof. The 5′ untranslated nucleotide may include a 5′ regulatory sequence as defined above. The regulatory sequence may be operably linked (as defined above) to the inventive cDNA sequence shown in SEQ ID NO: 2.

Nucleic acid molecules include DNA and RNA. The cloned nucleic acid molecule may include a cDNA clone. The DNA may be double-stranded or single-stranded, and if single stranded may be the coding strand or non-coding (anti-sense) strand. There are a number of methods which can be used to generate a cDNA clone that are well-known in the art. Using a nucleic acid having a sequence comprising all or part of the nucleic acid sequence of SEQ ID NO: 2 as a hybridization probe, nucleic acid molecules of the invention can be isolated using standard hybridization and cloning techniques. See, Sambrook et al., eds., Molecular Cloning: A Laboratory Manual, 2^{nd} ed., Cold Spring Harbor Laboratory, Cold Spring Harbor Laboratory Press, Cold Spring Harbor, N.Y. (1989).

Regardless of the pathway used to obtain the cDNA clone, the first step in cDNA cloning is the synthesis of a DNA strand complimentary to the mRNA sequence in a reaction requiring template RNA, a complimentary primer, reverse transcriptase, and the deoxyribonucleocide triphosphates. A cloned nucleic acid molecule according to this invention may further include a genomic clone. Moreover, a cloned nucleic acid molecule according to this invention may include subclones of genomic fragments or λ cDNA library clones. Subcloning cDNA from λ vectors into plasmid vectors, for example, enriches cDNA to vector mass ratios. There are many choices for useful plasmid vectors. Plasmids may possess promoters for phage (SP6, T7), RNA polymerases for in vitro transcription, multiple cloning sites, sequences complimentary to oligonucleotide primers for DNA sequencing, genes (β-galactosidase) for identifying clones by insertional inactivation, and properties to permit the direct isolation of the recombinant DNA in a single-stranded form.

A nucleic acid molecule of the invention can be amplified using cDNA, mRNA, or genomic DNA as a template and appropriate oligonucleotide primers according to standard PCR techniques. The nucleic acid so amplified can be cloned into an appropriate vector and characterized by DNA sequence analysis. Furthermore, oligonucleotides corresponding to all or part of a nucleic acid molecule of the invention can be prepared by standard synthetic techniques, i.e., using an automated DNA synthesizer.

Suitable methods for synthesizing DNA are described by Caruthers in Science 230:281-285 (1985) and “DNA Structure, Part A: Synthesis and Physical Analysis of DNA”, Lilley, D. M. J. and Dahlberg, J. E. (Eds.), Methods in Enzymology, 211, Academic Press, Inc., New York (1992). The subject matter of the aforementioned citations is herein incorporated by reference.

The invention further provides for a cloned nucleic acid molecule to be introduced into various cell types for in vivo expression of gene products. Specifically envisioned within the scope of the invention is a cloned mutant form of the polynucleotide of the invention which mediates or modulates the transmitter transport activity of the protein of the invention.

A cloned nucleic acid molecule according to this invention may have a number of uses. For example, the cloned sequences can be used to generate antibodies to the HNTTBMY1 protein. For example, cloned DNA can be ligated to a gene (e.g., β-galactosidase) such that both sequences are translated in-frame into a single polypeptide in Escherichia coli (E. coli); the fusion protein synthesized can then be purified and used as an antigen to generate polyclonal or monoclonal antibodies specific for the protein of interest. These aspects of the invention are further described below.

The invention also provides a cloned nucleic acid molecule encoding a fusion protein. Fusion proteins are discussed further below.

The present invention also encompasses polynucleotides capable of hybridizing, preferably under reduced stringency conditions, more preferably under stringent conditions, and most preferably under highly stringent conditions, to polynucleotides described herein. Examples of stringency conditions are shown in Table III below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE III

| Strin- | | | | |
|---|---|---|---|---|
| gency | Poly- | Hybrid | Hybridization | Wash |
| Con- | nucleotide | Length | Temperature | Temperature |
| dition | Hybrid± | (bp) ‡ | and Buffer† | and Buffer † |
| A | DNA:DNA | > or equal | 65(C; 1xSSC - | 65(C; 0.3xSSC |
| | | to 50 | or −42(C; | |
| | | | 1xSSC, 50% | |
| | | | formamide | |
| B | DNA:DNA | <50 | Tb*; 1xSSC | Tb*; 1xSSC |
| C | DNA:RNA | > or equal | 67(C; 1xSSC - | 67(C; 0.3xSSC |
| | | to 50 | or −45(C; | |
| | | | 1xSSC, 50% | |
| | | | formamide | |
| D | DNA:RNA | <50 | Td*; 1xSSC | Td*; 1xSSC |
| E | RNA:RNA | > or equal | 70(C; 1xSSC - | 70(C; 0.3xSSC |
| | | to 50 | or −50(C; | |
| | | | 1xSSC, 50% | |
| | | | formamide | |
| F | RNA:RNA | <50 | Tf*; 1xSSC | Tf*; 1xSSC |
| G | DNA:DNA | > or equal | 65(C; 4xSSC - | 65(C; 1xSSC |
| | | to 50 | or −45(C; | |
| | | | 4xSSC, 50% | |
| | | | formamide | |
| H | DNA:DNA | <50 | Th*; 4xSSC | Th*; 4xSSC |
| I | DNA:RNA | > or equal | 67(C; 4xSSC - | 67(C; 1xSSC |
| | | to 50 | or −45(C; | |
| | | | 4xSSC, 50% | |
| | | | formamide | |
| J | DNA:RNA | <50 | Tj*; 4xSSC | Tj*; 4xSSC |
| K | RNA:RNA | > or equal | 70(C; 4xSSC - | 67(C; 1xSSC |
| | | to 50 | or −40(C; | |
| | | | 6xSSC, 50% | |
| | | | formamide | |
| L | RNA:RNA | <50 | T1*; 2xSSC | T1*; 2xSSC |
| M | DNA:DNA | > or equal | 50(C; 4xSSC - | 50(C; 2xSSC |
| | | to 50 | or −40(C | |
| | | | 6xSSC, 50% | |
| | | | formamide | |
| N | DNA:DNA | <50 | Tn*; 6xSSC | Tn*; 6xSSC |
| O | DNA:RNA | > or equal | 55(C; 4xSSC - | 55(C; 2xSSC |
| | | to 50 | or −42(C; | |
| | | | 6xSSC, 50% | |
| | | | formamide | |
| P | DNA:RNA | <50 | Tp*; 6xSSC | Tp*; 6xSSC |
| Q | RNA:RNA | > or equal | 60(C; 4xSSC - | 60(C; 2xSSC |
| | | to 50 | or −45(C; | |
| | | | 6xSSC, 50% | |
| | | | formamide | |
| R | RNA:RNA | <50 | Tr*; 4xSSC | Tr*; 4xSSC |
| ‡The “hybrid length” is the anticipated length for the hybridized region(s) of the hybridizing polynucleotides. When hybridizing a polynucleotide of unknown sequence, the hybrid is assumed to be that of the hybridizing polynucleotide of the present invention. When polynucleotides of known sequence are hybridized, the hybrid length can be determined by aligning the sequences of | | | | |
| #the polynucleotides and identifying the region or regions of optimal sequence complementarity. Methods of aligning two or more polynucleotide sequences and/or determining the percent identity between two polynucleotide sequences are well known in the art (e.g., MegAlign program of the DNA*Star suite of programs, etc). | | | | |
| †SSPE (1xSSPE is 0.15 M NaCl, 10 mM NaH2PO4, and 1.25 mM EDTA, pH 7.4) can be substituted for SSC (1xSSC is 0.15 M NaCl and 15 mM sodium citrate) in the hybridization and wash buffers; washes are performed for 15 minutes after hybridization is complete. The hydridizations and washes may additionally include 5X Denhardt's reagent, .5-1.0% SDS, 100 ug/ml denatured, fragmented | | | | |
| #salmon sperm DNA, 0.5% sodium pyrophosphate, and up to 50% formamide. | | | | |
| *Tb-Tr: The hybridization temperature for hybrids anticipated to be less than 50 base pairs in length should be 5-10(C less than the melting temperature Tm of the hybrids there Tm is determined according to the following equations. For hybrids less than 18 base pairs in length, Tm((C) = 2(# of A + T bases) + 4(# of G + C bases). For hybrids between 18 and 49 base | | | | |
| #pairs in length, Tm((C) = 81.5 + 16.6(log₁₀[Na+]) + 0.41(% G + C) − (600/N), where N is the number of bases in the hybrid, and [Na+] is the concentration of sodium ions in the hybridization buffer ([NA+] for 1xSSC = .165 M). | | | | |

±The present invention encompasses the substitution of any one, or more DNA or RNA hybrid partners with either a PNA, or a modified polynucleotide. Such modified polynucleotides are known in the art and are more particularly described elsewhere herein.

Additional examples of stringency conditions for polynucleotide hybridization are provided, for example, in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M., Ausubel et al., eds, John Wiley and Sons, Inc., sections 2.10 and 6.3-6.4, which are hereby incorporated by reference herein.

Preferably, such hybridizing polynucleotides have at least 70% sequence identity (more preferably, at least 80% identity; and most preferably at least 90% or 95% identity) with the polynucleotide of the present invention to which they hybridize, where sequence identity is determined by comparing the sequences of the hybridizing polynucleotides when aligned so as to maximize overlap and identity while minimizing sequence gaps. The determination of identity is well known in the art, and discussed more specifically elsewhere herein.

### Polypeptides of the Invention

The invention further provides an isolated or recombinant human polypeptide, comprising the amino acid sequence of SEQ ID NO: 1 or a fragment or mutant form thereof.

The inventive protein, biologically active portions thereof, as well as fragments thereof may be suitable for use as immunogens to raise antibodies directed against a polypeptide of the invention. The human polypeptides of the present invention and DNA encoding the polypeptide may be isolated from natural sources, chemically synthesized, or recombinantly produced by methods known in the art. Suitable methods for synthesizing the protein are described by Stuart and Young in Solid Phase Peptide Synthesis, Second Edition, Pierce Chemical Company (1984); Solid Phase Peptide Synthesis, Methods in Enzymology, 289, Academic Press, Inc, New York (1997). The subject matter of the aforementioned citations is herein incorporated by reference.

A recombinant process involves providing DNA that encodes the protein; amplifying or cloning the DNA in a suitable host; expressing the DNA in a suitable host; and harvesting the protein. For example, the HNTTBMY1 polypeptide or a fragment thereof may be translated either directly or indirectly from the cDNA encoding all or part of the amino acid sequence.

The HNTTBMY1 protein and fragments thereof are preferably isolated. An “isolated” protein or polypeptide or fragment thereof is substantially free of cellular material or other contaminating proteins from the cell or tissue source from which the protein is derived so that it is partially purified or purified to homogenicity. The protein is considered partially purified if it is at least 25%, preferably at least approximately 50%, more preferably at least 75%, most preferably at least 90%, and optimally at least approximately 95% free of other cellular material and/or contaminating proteins. The protein or fragment thereof is considered to be purified to homogeneity if it exhibits a single band by SDS page. If chemically synthesized, the protein is “isolated” if it is substantially free of chemical precursors or other chemicals. If produced through recombinant technology, the protein is “isolated” if it is substantially free of culture medium.

The invention includes functional equivalents of the HNTTBMY1 protein, its fragments, and mutants. Preferably, a human protein or fragment thereof is a functional equivalent if its amino acid sequence is at least approximately 60% identical, more preferably at least approximately 70% identical.

The determination of percent identity between two sequences can be accomplished using a mathematical algorithm. One example is the algorithm as described by Altschul, S. F. in J. Mol. Evol., 36:290-300 (1993). Such an algorithm is incorporated into the NBLAST and XBLAST programs of Altschul, et al. described in J. Mol. Biol., 215:403-410 (1990). BLAST (Basic Local Alignment Search Tool) nucleotide searches can be performed with the NBLAST program, score=100, wordlength=12 to obtain nucleotide sequences homologous to a nucleic acid molecule of the invention. BLAST protein searches can be performed with the XBLAST program, score=50, wordlength=3 to obtain amino acid sequences homologous to a protein molecule of the invention. Software for performing BLAST analyses is publicly available through the National Center for Biotechnology Information (http://www.ncbi.nlm.nih.gov).

Furthermore, Altschul, S. F., et al. describe “Gapped BLAST and PSI-BLAST, a new generation of protein database search programs,” in Nucleic Acids Res., 25:3389-3402, (1997).

Another program, Genewise, compares genomic sequences to one or more protein reference sequences, or to Hidden Markov Models (HMM's) representing protein domains. It performs the comparison at the protein translation level, while simultaneously maintaining a reading frame regardless of intervening introns and sequencing errors, which may otherwise cause frame shifts.

To obtain gapped alignments for comparison purposes, an algorithm based on the progressive pairwise alignments to show relationship and percent sequence identity, may be used. Such an algorithm, for example, is incorporated in the PILEUP program, a simplification of the algorithm as described by Feng and Doolittle, J. Mol. Evol., 25:351-360 (1987). Another gap alignment algorithm providing percent identity and similar values is incorporated in GAP (Global Alignment Program) as described in Needleman and Wunsch, J. Mol. Bio., 48(3):443-454 (1970). (Genetics Computer Group, Princeton, N.J.). Another alignment algorithm includes both multiple sequence alignments and theoretical hidden Markov models (HMM's) of protein domains referred to as the PFAM protein families database. The alignments represent certain evolutionarily conserved structures. Bateman, E. et al., Nucleic Acid Research, 28:263-266 (2000). Such an algorithm is incorporated into the PFAM program (version 6.6 dated August 2001) available through the Sanger Institute website (http:/Hwww.sanger.ac.uk/Software/Pfam/). A further algorithm predicts membrane spanning regions and their orientation, based on statistical analysis of a database of naturally occurring TM proteins [Hofmann K., and Stoffel; W., Biol. Chem. Hoppe-Seyler, 347:166, (1993)]. This algorithm is the basis for TMPRED which analyzes protein sequences predicting the occurrence of TM domains.

A preferred algorithm for calculating percent identity between two polynucleotides and/or polypeptides is the CLUSTALw algorithm (Thompson, J. D., et al., Nucleic Acids Research, 2(22):4673-4680, (1994)). The algorithm is preferred since it takes into account differences in lengths of the two sequences, in addition to gaps in either sequence in assessing the overall percent identity.

As a practical matter, whether any particular nucleic acid molecule or polypeptide is at least about 80%, 85%, 90%, 91%, 92%, 93%, 94%, 95%, 96%, 97%, 97.1%, 98%, 98.2%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. Manual corrections for global percent identity determinations are required since the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, 85%, 90%, 91%, 92%, 93%, 94%, 95%, 96%, 97%, 97.1%, 98%, 99%, 99.1%, 99.2%, 99.3%, 99.4%, 99.5%, 99.6%, 99.7%, 99.8%, or 99.9% identical to, for instance, an amino acid sequence referenced in Table II (SEQ ID NO: 1) or to the amino acid sequence encoded by cDNA contained in a deposited clone, can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both amino acid sequences. The result of said global sequence alignment is in percent identity. Preferred parameters used in a CLUSTALW alignment of polypeptide sequences to calculate percent identity via pairwise alignments are: Matrix=BLOSUM, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of N- or C-terminal deletions, not because of internal deletions. If only the local pairwise percent identity is required, no manual correction is needed. However, a manual correction may be applied to determine the global percent identity from a global polypeptide alignment. Percent identity calculations based upon global polypeptide alignments are often preferred since they reflect the percent identity between the polypeptide molecules as a whole (i.e., including any polypeptide overhangs, not just overlapping regions), as opposed to, only local matching polypeptides. Manual corrections for global percent identity determinations are required since the CLUSTALW program does not account for N- and C-terminal truncations of the subject sequence when calculating percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

In addition to the above method of aligning two or more polynucleotide or polypeptide sequences to arrive at a percent identity value for the aligned sequences, it may be desirable in some circumstances to use a modified version of the CLUSTALW algorithm which takes into account known structural features of the sequences to be aligned, such as for example, the SWISS-PROT designations for each sequence. The result of such a modifed CLUSTALW algorithm may provide a more accurate value of the percent identity for two polynucleotide or polypeptide sequences. Support for such a modified version of CLUSTALW is provided within the CLUSTALW algorithm and would be readily appreciated to one of skill in the art of bioinformatics.

It is within the contemplation of the invention that the polypeptide of the invention may include conservative substitutions of amino acids of the protein. Groups of amino acids known to normally be equivalent are:

a. Ala (A), Ser (S), Thr (T), Pro (P), Gly (G);

b. Asn (N), Asp (D), Glu (E), Gln (Q);

c. His (H), Arg (R), Lys (K);

d. Met (M), Leu (L), Ile (I), Val (V), and

e. Phe (F), Tyr (Y), Trp (W).

The invention encompasses polypeptides having a lower degree of identity but having sufficient similarity so as to perform one or more of the same functions performed by the polypeptide of the present invention. Similarity is determined by conserved amino acid substitution. Such substitutions are those that substitute a given amino acid in a polypeptide by another amino acid of like characteristics (e.g., chemical properties). According to Cunningham et al above, such conservative substitutions are likely to be phenotypically silent. Additional guidance concerning which amino acid changes are likely to be phenotypically silent are found in Bowie et al., Science 247:1306-1310 (1990).

Tolerated conservative amino acid substitutions of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In addition, the present invention also encompasses the conservative substitutions provided in Table IV below.

TABLE IV

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-1-thioazolidine-4-carboxylic acid, |
| | | D- or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Aside from the uses described above, such amino acid substitutions may also increase protein or peptide stability. The invention encompasses amino acid substitutions that contain, for example, one or more non-peptide bonds (which replace the peptide bonds) in the protein or peptide sequence. Also included are substitutions that include amino acid residues other than naturally occurring L-amino acids, e.g., D-amino acids or non-naturally occurring or synthetic amino acids, e.g., B or (amino acids.

Both identity and similarity can be readily calculated by reference to the following publications: Computational Molecular Biology, Lesk, A. M., ed., Oxford University Press, New York, 1988; Biocomputing: Informatics and Genome Projects, Smith, D. W., ed., Academic Press, New York, 1993; Informatics Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991.

In addition, the present invention also encompasses substitution of amino acids based upon the probability of an amino acid substitution resulting in conservation of function. Such probabilities are determined by aligning multiple genes with related function and assessing the relative penalty of each substitution to proper gene function. Such probabilities are often described in a matrix and are used by some algorithms (e.g., BLAST, CLUSTALW, GAP, etc.) in calculating percent similarity wherein similarity refers to the degree by which one amino acid may substitute for another amino acid without lose of function. An example of such a matrix is the PAM250 or BLOSUM62 matrix.

Aside from the canonical chemically conservative substitutions referenced above, the invention also encompasses substitutions which are typically not classified as conservative, but that may be chemically conservative under certain circumstances. Analysis of enzymatic catalysis for proteases, for example, has shown that certain amino acids within the active site of some enzymes may have highly perturbed pKa's due to the unique microenvironment of the active site. Such perturbed pKa's could enable some amino acids to substitute for other amino acids while conserving enzymatic structure and function. Examples of amino acids that are known to have amino acids with perturbed pKa's are the Glu-35 residue of Lysozyme, the Ile-16 residue of Chymotrypsin, the His-159 residue of Papain, etc. The conservation of function relates to either anomalous protonation or anomalous deprotonation of such amino acids, relative to their canonical, non-perturbed pKa. The pKa perturbation may enable these amino acids to actively participate in general acid-base catalysis due to the unique ionization environment within the enzyme active site. Thus, substituting an amino acid capable of serving as either a general acid or general base within the microenvironment of an enzyme active site or cavity, as may be the case, in the same or similar capacity as the wild-type amino acid, would effectively serve as a conservative amino substitution.

On the basis of the positioning of various domain regions potentially important for HNTTBMY1's function and regulation, it is well within the contemplation of the present invention that only a fragment of the HNTTBMY protein may be required for functional activity.

In a preferred embodiment, the functional activity displayed by a polypeptide encoded by a polynucleotide fragment of the invention may be one or more biological activities typically associated with the full-length polypeptide of the invention. Illustrative of these biological activities includes the fragments ability to bind to at least one of the same antibodies which bind to the full-length protein, the fragments ability to interact with at lease one of the same proteins which bind to the full-length, the fragments ability to elicit at least one of the same immune responses as the full-length protein (i.e., to cause the immune system to create antibodies specific to the same epitope, etc.), the fragments ability to bind to at least one of the same polynucleotides as the full-length protein, the fragments ability to bind to a receptor of the full-length protein, the fragments ability to bind to a ligand of the full-length protein, and the fragments ability to multimerize with the full-length protein. However, the skilled artisan would appreciate that some fragments may have biological activities which are desirable and directly inapposite to the biological activity of the full-length protein. The functional activity of polypeptides of the invention, including fragments, variants, derivatives, and analogs thereof can be determined by numerous methods available to the skilled artisan, some of which are described elsewhere herein.

As will be appreciated by the skilled practitioner, should the amino acid fragment comprise an antigenic epitope, for example, biological function per se need not be maintained. The terms HNTTBMY1 polypeptide and HNTTBMY1 protein are used interchangeably herein to refer to the encoded product of the HNTTBMY1 nucleic acid sequence according to the present invention.

The invention also provides a chimeric or fusion protein. As used herein, a fusion protein comprises all or part (preferably a biologically active part) of a polypeptide of the invention operably linked to a heterologous or unrelated polypeptide. The unrelated polypeptide may be a detectable label for enabling detection of the polypeptide of the invention or a matrix-binding domain for immobilizing the fusion protein. The fusion proteins can be produced by standard recombinant DNA techniques.

### Oligonucleotides and Primers

In yet another aspect of the present invention, a substantially pure oligonucleotide probe or primer is provided, wherein the oligonucleotide probe or primer includes a region of nucleotide sequence capable of hybridizing under stringent conditions to at least about 12 consecutive nucleotides of sense or anti-sense sequence of a human polynucleotide represented by SEQ ID NO: 2 or a mutant form thereof. In a further embodiment, a substantially pure oligonucleotide is provided, wherein the oligonucleotide includes a region of nucleotide sequence capable of hybridizing under stringent conditions, to at least about 12 consecutive nucleotides of sense or anti-sense sequence of a non-coding nucleotide sequence 5′ or 3′ of the coding sequence for the polypeptide of SEQ ID NO: 1. In one embodiment, the oligonucleotide or primer hybridizes to mutant forms of the 5′ or 3′ untranslated region. In a further embodiment, the oligonucleotide or primer hybridizes to at least about 12 consecutive nucleotides of sense or anti-sense sequence of the 5′ untranslated region represented by SEQ ID NO: 3. Examples of suitable oligonucleotides according to the invention are represented by SEQ ID NO: 4, SEQ ID NO: 5, SEQ ID NO: 8, SEQ ID NO: 9, SEQ ID NO: 10, and SEQ ID NO: 11.

Also encompassed by the present invention are oligonucleotides or primers as described above wherein the oligonucleotide or primer further includes a detectable label attached thereto.

There is no upper limit to the length of the oligonucleotide probes or primers. However, normally, the oligonucleotide probe will not contain more than 50 nucleotides, preferably not more than 40 nucleotides, and more preferably not more than 30 nucleotides.

The oligonucleotides and primers provided by the present invention may be useful for diagnosing a particular disorder, such as by PCR amplification of portions of the HNTTBMY1 nucleotide sequence which may harbor a mutation, insertion, or deletion of a nucleotide base, as one example. Moreover, the probes and primers provided herein may be used as part of anti-sense therapy. This is further described below and relates to a potential therapeutic use of such oligonucleotide probes or primers.

### Expression Vectors

The invention provides for an expression vector or fragments or mutant forms thereof capable of expressing in a host cell the HNTTBMY1 protein described above. In one embodiment, an expression vector according to this invention allows for the translation of protein domains which are capable of facilitating the function of HNTTBMY1 to affect various cellular processes associated with neurotransmitter transport, including but not limited to, Na⁺/Cl⁻ dependent signal transport. The invention includes expression vectors that are capable of mediating or modulating a transmitter transporter activity of a protein of the invention.

The DNA encoding the protein of the invention may be replicated and used to express recombinant protein following insertion into a wide variety of host cells in a wide variety of cloning vectors. The term “vector” as used herein refers to a nucleic acid molecule capable of transporting another nucleic acid to which it has been linked. One type of vector is a “plasmid,” which is a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (i.e., bacetrial vectors having a bacterial origin of replication and episomal mammalian vectors). Other vectors (i.e., non-episomal mammalian vectors) are integrated into the genome of a host cell upon introduction into the host cell, and thereby are replicated along with the host genome. Moreover, expression vectors are capable of directing the expression of genes to which they are operably linked. In general, expression vectors useful in recombinant DNA techniques are often in the form of plasmids. However, the invention is intended to include other forms of expression vectors, such as viral vectors (i.e., replication defective retroviruses, adenoviruses and adeno-associated viruses) that serve equivalent functions.

The host may be prokaryotic or eukaryotic. A DNA may be obtained from natural sources and, optionally, modified. The genes may also be synthesized in whole or in part. Cloning vectors may comprise segments of chromosomal, non-chromosomal, and synthetic DNA sequences.

Some suitable prokaryotic cloning vectors include plasmids from E. coli, such as col E1, pCR 1, pBR 322, pMB9, pUC, pKSM, and RP4. Prokaryotic vectors also include derivatives of phage DNA such as M13 fd and other filomentuous single-stranded DNA phages. Vectors for expressing proteins in bacteria, especially E. coli, are also known. Such vectors include the pK 233 (or any of the tac family of plasmids), T7, pBluescript II, Bacteria phase lambda, Zap, and lambda P_{1.} Wu, R. (Ed), Recombinant DNA Methodology II, Methods in Enzymology, Academic Press, Inc., N.Y., (1999-1995). Examples of vectors with expressed fusion proteins are PATH vectors described by Dieckmann and Tzagoloff in J. Biol. Chem., 260:1513-1520 (1985). These vectors contain DNA sequences that encode anthranilate synthetase (TrpE) followed by a poly-linker at the carboxy terminus. Other expression vector systems are based on beta-galactosidase (pEX); maltose binding protein (pMAL); glutathionestranferase (pGST or pGEX)—see Smith, D. B. Methods Mol. Cell Biol., 4:220-229 (1993); Smith, D. B. and Johnson, K. S., Gene, 67:31-40 (1988); and Peptide Res., 3:167 (1990); and TRX (thioredoxin) Fusion Protein (TRX FUF)—see La Vallie, R. et al., Bio/Technology, 11:187-193 (1993).

Suitable cloning/expression vectors for use in mammalian cells are also known. Such vectors include well-known derivatives of SV-40, adenovirus, cytomegalovirus (CMV) retrovirus-derived DNA sequences. Any such vectors, when coupled with vectors derived from a combination of plasmids and phage DNA, i.e. shuttle vectors, allow for the isolation and identification of protein coding sequences in prokaryotes.

Further eukaryotic expression vectors are known in the art (e.g., P. J. Southern and P. Berg, J. Mol. Appl. Genet. 1:327-341 (1982); S. Subramani et al, Mol. Cell. Biol. 1:854-864 (1981); Kaufmann and Sharp, “Amplification And Expression Of Sequences Cotransfected with A Modular Dihydrofolate Reductase Complementary DNA Gene,” J. Mol. Biol., 159:601-621 (1982); Kaufmann, R. J. and Sharp, P. A., Mol. Cell. Biol., 159:601-664 (1982); Scahill, S. I., et al., “Expression And Characterization Of The Product Of A Human Immune Interferon DNA Gene In Chinese Hamster Ovary Cells,” Proc. Natl. Acad. Sci. USA, 80:4654-4659 (1983); Urlaub, G. and Chasin, L. A., Proc. Natl. Acad. Sci. USA, 77:4216-4220 (1980).

Vectors useful for cloning and expression in yeast are available. Suitable examples are 2 μm circle plasmid, Ycp50, Yep24, Yrp7, Yip5, and pYAC3. Ausubel, F. M. et al., eds., Current Protocols in Molecular Biology, John Wiley & Sons, Inc., New York, (1999).

Vector DNA can be introduced into cells via conventional transformation or transfection techniques. As used herein, the terms “transformation” and “transfection” are intended to refer to various art-recognized techniques for introducing foreign nucleic acid into a host cell, including calcium phosphate or calcium chloride co-precipitation or electroporation. Suitable methods for transforming or transfecting host cells can be found, for example, in Sambrook, et al. (supra). The present invention includes a host cell transfected with an expression vector of the invention.

Usually only a small fraction of cells integrate the foreign DNA into their genome. In order to identify and select these host cells, a gene that encodes a selectable marker, for example a protein necessary for survival or growth of a host cell transformed with the vector, is introduced into the host cells along with the gene of interest. The presence of this gene insures growth of a host cell transformed or transfected with the vector allowing growth and selection of only those host cells which express the insert. Typical selection genes encode proteins that: (a) confer resistance to antibiotics or other toxic substances (i.e. ampicillin, neomycin, methotrexate, etc.); (b) compliment auxotrophec deficiencies or (c) supply critical nutrients not available from complex media, i.e., gene encoding d-alanine, racenase for Basillia. The choice of the proper selectable marker will depend on the host cell, and appropriate markers for different hosts are well known in the art.

The expression vectors useful in the present invention contain at least one expression control sequence that is operably linked to the DNA sequence or fragment to be expressed. The control sequence is inserted in the vector in order to control and to regulate the expression of the cloned DNA sequence. Examples of useful expression control sequences are the lac system, the trp system, the tac system, the trc system, the tet system, major operator and promoter regions of phage lambda, the control region of fd coat protein, the glycolytic promoters of yeast, e.g., the promoter for 3-phosphoglycerate kinase, the promoters of yeast acid phosphatase, e.g., Pho5, the promoters of the yeast alpha-mating factors, and promoters derived from polyoma, adenovirus, retrovirus, and simian virus, e.g., the early and late promoters or SV40, and other sequences known to control the expression of genes of prokaryotic or eukaryotic cells and their viruses or combinations thereof. Regulatory sequences are described, for example, in Goeddel, Gene Expression Technology: Methods in Enzymology 185, Academic Press, San Diego, Calif. (1990).

Once the gene is cloned into such an expression vector, the gene product may be produced, for example in E. coli, in either a constitutive or inducible manner. Useful expression hosts include well-known prokaryotic and eukaryotic cells. Some suitable prokaryotic hosts include, for example, E. coli, such as E. coli SG-936, E. coli HB 101, E. coli W3110, E. coli X1776, E. coli X2282, E. coli DH1, E. coli DH5αF′, and E. coli MRCl, Pseudomonas, Bacillus, such as Bacillus subtilis, and Streptomyces. Suitable eukaryotic cells include yeasts and other fungi, insect, animal cells, such as COS cells and CHO cells, human cells and plant cells in tissue culture. The expressed protein can be purified using methods well known in the art.

### Host Cells

The invention provides for a host cell transfected with the expression vector of the present invention, which host cell is capable of expressing a polypeptide including the amino acid sequence of SEQ ID NO: 1, or a fragment thereof in a functional and/or mutant form. Host cells which contain and express the nucleic acid sequence encoding HNTTBMY1 may be identified by a variety of procedures known to those skilled in the art. These procedures include, but are not limited to, DNA-DNA or DNA-RNA hybridization and protein bioassay or immunoassay techniques which include membrane and solution based technologies for the detection and/or quantification of nucleic acids or proteins.

### Method of Making HNTTBMY1 Protein

The invention further provides a method of making a HNTTBMY1 polypeptide or fragment or mutant thereof. The method includes: (a) culturing a host cell as described above under conditions suitable for the expression of the polypeptide or fragment thereof; and (b) recovering the polypeptide or fragment thereof from the host cell culture.

The polypeptides may be purified using standard known techniques. Some examples of suitable techniques include, for example, gel purification, column chromatography, or electrophoretic methods. Recombinant constructions may be used to join sequences encoding HNTTBMY1 to nucleic acid sequence encoding a polypeptide domain which will facilitate purification of soluble proteins. Such purification facilitating domains include, but are not limited to, metal chelating peptides such as histidine-tryptophan modules that allow purifiction on immobilized metals, protein A domains that allow purification or immobilized immunoglobulin, and the domain utilized in the flags-extension/affinity purification system (Immunex Corp., Seattle, Wash.). Moreover, signal sequences may be used to facilitate the export of HNTTBMY1 into a cell culture supernatant to facilitate purification of the protein.

### Method of Use: Peptide Therapy and Gene Therapy

Peptides, inclusive of polypeptides which have HNTTBMY1 activity, can be supplied to cells which carry a mutated HNTTBMY1 gene. The sequence of the human HNTTBMY1 protein is disclosed in SEQ ID NO: 1. Protein can be produced by expression of cDNA sequence in bacteria, for example, using known expression vectors as described above. Alternatively, HNTTBMY1 polypeptide can be extracted from HNTTBMY1-producing mammalian cells as described above. Moreover, the techniques of synthetic chemistry can be employed to synthesize HNTTBMY1 protein. Any such techniques can provide the preparation of the present invention which includes the HNTTBMY1 protein. The preparation is substantially free of other human proteins. This can be most readily accomplished by synthesis of the protein in a micro-organism or in vitro. The preparation is a pharmaceutical composition.

The pharmaceutical composition including the HNTTBMY1 protein or peptide may be administered in conjunction with a pharmaceutically acceptable carrier, such as clinical grade sterile water. Moreover, such compositions may, in addition to including HNTTBMY1 derivatives, further include agonists or mimetics of HNTTBMY1. The pharmaceutical compositions utilized in this invention may be administered by any number of routes including, but not limited to, oral, intravenous, intra-muscular, intra-arterial, intra-medullary, intra-thecal, intra-ventricular, transdermal, subcutaneous, intra-peritoneal, intra-nasal, enteral, topical, sublingual, or rectal means. Active HNTTBMY1 polypeptides or derivatives thereof can be introduced into cells by micro-injection or by use of liposomes, for example. Alternatively, some active molecules may be taken up by cells actively or by diffusion.

The invention provides a method of modulating neurotransmitter transporter activity of a neurotransmitter transporter. The method includes introducing into the cell an isolated or recombinant polypeptide comprising the amino acid sequence of SEQ ID NO: 1 or a fragment analog or mutant form thereof. As a result, the present invention provides methods of treating an individual afflicted with a disease or disorder characterized by aberrant expression or activity of a polypeptide of the invention.

In preferred embodiments, the cells are derived from brain or spinal cord tissue. In particular, a brain cell may be derived from the following brain subregions: amygdala, caudate nucleus, cerebellum, corpus callosum, hippocampus, substantia nigra, thalamus, and combinations thereof.

In one aspect of this method, the HNTTBMY1 polypeptide is introduced into the cell by expressing a nucleic acid molecule that encodes HNTTBMY1 polypeptide in the cell. Alternatively, the HNTTBMY1 polypeptide may be introduced into the cell by contacting the cell with a polynucleotide form of the invention which encodes the polypeptide of the invention.

Active HNTTBMY1 molecules can be introduced into cells by micro-injection or by use of liposomes. Alternatively, some active molecules may be taken up by cells, actively or by diffusion. Extracellular application of the HNTTBMY1 polypeptide may also be sufficient to affect neurotransmitter transport. The HNTTBMY1 polypeptide for use in the method described above is that of a human.

In one preferred aspect, the polypeptide includes approximately amino acids from 1 to the C-terminus. For example, one useful polypeptide would be the human HNTTBMY1 protein represented in SEQ ID NO: 1. The method above further encompasses the use of a HNTTBMY1 protein fragment. Preferably, the fragment would be capable of facilitating the normal functions of HNTTBMY1 within the cell.

Yet another object of the invention is to provide compositions comprising N-terminal, C-terminal or internal deletion polypeptides of the encoded HNTTBMY1 polypeptide. Polynucleotides encoding these deletion polypeptides are also provided. The present invention also provides the use of these polypeptides as an immunogenic and/or antigenic epitope as described elsewhere herein.

Based on the various domains found in the amino acid sequence for the HNTTBMY1 protein, fragments of the protein which may be useful in the method described above, include those segments of the polypeptide sequence that reside in the TM domains. These are expected to be relevant to the proper functioning of the protein within the cell, or an analog thereof.

The present invention also encompasses a method of supplying a wild-type HNTTBMY1 nucleic acid form or a functional analog thereof to a cell which carries a mutated form of a gene. For example, a HNTTBMY1 gene, or part thereof, may be introduced into the cell in a vector such that the gene remains extrachromosomal. More preferred, is a situation where the wild-type HNTTBMY1 gene, or a part thereof, is introduced into the mutant cell in such a way that it recombines with the endogenous mutant HNTTBMY1 gene present in the cell. Methods for introducing DNA into cells such as electroporation, calcium phosphate coprecipitation and viral transduction are known in the art. Cells transformed with the wild-type HNTTBMY1 gene can be used as model systems to study neurotransmitter transport and drug treatments which promote or modulate such transport.

As generally discussed above, the HNTTBMY1 gene or fragment, where applicable, may be employed in gene therapy methods in order to increase the amount of the expression products of such genes. Such gene therapy may be particularly appropriate for use in conditions in which the level of a functional HNTTBMY1 polypeptide is absent or diminished compared to normal cells. Gene therapy is carried out according to generally accepted methods, for example, as described in Friedman (Ed.), Therapy for Genetic Disease, Oxford University Press, London, UK (1991). Briefly, a patient's mutated cells are first analyzed to decipher the production of HNTTBMY1 polypeptide in the mutated cells. A virus or plasmid vector containing a copy of the HNTTBMY1 gene is prepared including expression control elements incapable of replicating inside the mutated cells. Suitable vectors include those disclosed in U.S. Pat. No. 5,252,479 as well as PCT Published Application WO 93/07282, the subject matter of which are herein incorporated by reference. The vector is injected into the patient, either locally or systemically.

In a further aspect, anti-sense polynucleotide sequences are useful for modulating HNTTBMY1 activity or to achieve regulation of gene function. To this end, anti-sense to nucleic acid sequences encoding HNTTBMY1 may be used in situations in which it would be desirable to block the transcription of the mRNA. In particular, cells may be transformed with sequences complimentary to nucleic acid sequences encoding HNTTBMY1. Such technology is now well known in the art, and sense or anti-sense oligomers or larger fragments, can be designed for various locations along the coding or control regions of sequences encoding HNTTBMY1. Antisense oligonucleotides may be single or double stranded. Double stranded RNA's may be designed based upon the teachings of Paddison et al., Proc. Nat. Acad. Sci., 99:1443-1448 (2002); and International Publication Nos. WO 01/29058, and WO 99/32619; which are hereby incorporated herein by reference.

In another aspect of the invention, there is provided a method of modulating neurotransmitter transporter activity of a neurotransmitter transporter that includes introducing into the cell an agonist of a nucleic acid form encoding the polypeptide of SEQ ID NO: 1 or an agonist of amino acids encoded by the nucleic acid form. In a particular aspect of the invention, this method involves modulating the electrogenic ion coupled transporter activity and/or sodium dependent “channel mode” neurotransmitter transport associated with the neurotransmitter transporter according to the present invention. An “agonist” is defined herein as any substance employed to enhance the biological activities (e.g. neurotransmitter transport activity) mediated through polypeptides of the present invention.

In a further aspect of the invention there is provided a method of modulating the transmitter transporter activity of a neurotransmitter transporter, the method comprising introducing into a cell an antagonist of a nucleic acid form encoding a human polypeptide of SEQ ID NO: 1 or an antagonist of amino acids encoded by the nucleic acid form. In one embodiment, this method involves modulating the electrogenic ion coupled transporter activity and/or sodium dependent “channel mode” neurotransmitter transport associated with the inventive transporter.

Antagonists of both nucleic acid and protein forms of HNTTBMY1 (inclusive of mutant forms) provide a means by which the functional activity may be controlled both at a nucleic acid and protein level. In one aspect, the antagonist of the HNTTBMY1 nucleic acid form is an anti-sense construct. For example, anti-sense oligomers which are oligomers complimentary in sequence to the cDNA sequence coding for HNTTBMY1 may be used to antagonize the activity of one or more domains and to interrupt neurotransmitter transport.

The length of the antisense oliglionucleotide is not critical, as long as it is capable of hybridizing to a region of the cDNA which encodes for HNTTBMY 1. The antisense oliglionucleotide should contain at least 6 nucleotides, preferably at least 10 nucleotides, and, more preferably, at least 15 nucleotides. There is no upper limit to the length of the oliglionucleotide probes. Longer probes are more difficult to prepare and require longer hybridization times. Therefore, the probe should not be longer than necessary. Normally, the oliglionucleotide probe will not contain more than 50 nucleotides, preferably not more than 40 nucleotides, and more preferably, not more than 30 nucleotides.

In a further aspect of the invention, the antagonist of the HNTTBMY1 nucleic acid form is a peptide antagonist.

In a still further aspect, the antagonist is a mutant polynucleotide of the invention.

Moreover, it is within the contemplation of the present invention that a mutant HNTTBMY1 form, could result in a block to the ability of cells to transport neurotransmitters by HNTTBMY1. Further, an antagonist against such a mutant HNTTBMY1 form, could further result in a blockade of the effect of the mutant form, and be able to restore the neurotransmitter transporter activity of HNTTBMY1. Therefore, therapeutic agents can be designed against the truncated forms or other mutant forms of HNTTBMY1.

Moreover, antagonists of amino acids encoded by the HNTTBMY1 nucleic acid form include a dominant negative version of the HNTTBMY1 protein or a hormone-inducible or drug-inducible version thereof. In one example, spinal cord tissue or brain tissue is injected with DNA or RNA, such as mRNA, which, when translated, forms an inactive version of the HNTTBMY1 protein capable of blocking the ability of HNTTBMY1 wild-type protein to perform neurotransmitter transport.

In a further aspect, the antagonist of amino acids encoded by the HNTTBMY nucleic acid form is a monoclonal antibody. The monoclonal antibody provided herein is further described below.

The cell for use in the agonist or antagonist methods just described may be from at least one of the brain or the spinal cord. If from brain, the cell may be from at least one of the subregions of the brain including anygdala, caudate nucleus, cerebellum, corpus callosum, hippocampus, substantia nigra, and thalamus.

It is noted that any of the therapeutic proteins, antagonists, antibodies, agonists, anti-sense sequences, or vectors described above may be administered in combination with other appropriate therapeutic agents. Selection of the appropriate agents for use in combination therapy may be made by one of ordinary skill in the art. This combination of therapeutic agents may act in a synergistic manner to effect the treatment or prevention of the various disorders described above. Using this approach, therapeutic efficacy with lower doses of each agent may be achieved, thus reducing the potential for adverse side effects.

HNTTBMY1 polynucleotides, polypeptides, fragments, and modulators thereof are useful for detecting, treating, and/or ameliorating a variety of disorders, particularly of the central and peripheral nervous system. Such disorders, include, but are not limited to the following: behavioral disorders; memory disorders; cognitive disorders; disorders associated with aberrant serotonin expression and/or activity; anxiety, fear, depression, sleep, pain, disorders associated with aberrant maintenance of an attentive or alert state; attention deficit disorders; disorders affecting the ‘reward center’ of the brain; disorders affecting the synthesis, and/or effecting the release of neurotransmitters such as dopamine, opioid peptides, serotonin, GABA, and glutamate; addictive disorders; homeostatic disorders; neuroendocrine disorders; disorders affecting the establishment of long term potentiation; circadian rhythm disorders; disorders associated with the establishment of aberrant sleep/wake cycles; dopaminergic functional disorders; neuronal transmission system disorders, and pain.

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human. immunodeficiency virus-associated myelopathy, transverse myelopathy or various etiologies, progressive multifocal leukoencephalopathy, and central pontine myelinolysis.

In a preferred embodiment, the polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

A polypeptide of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins. minerals, cofactors or other nutritional factors or component(s); effecting behavioral characteristics, including, without limitation, appetite, libido, stress, cognition (including cognitive disorders), depression (including depressive disorders) and violent behaviors, analgesic effects or other pain reducing effects; promoting differentiation and growth of embryonic stem cells in lineages other than hematopoletic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

### Antibodies

In a preferred aspect of the invention, the antagonist of amino acids encoded by the gene sequence is a monoclonal antibody. This antibody can be a derivative of an antibody such as a humanized antibody, a chimerized antibody or a fragment of an antibody that contains a binding site which would recognize all or part of either the gene or a domain of the protein sufficient to modulate transport of neurotransmitters. An “antibody” in accordance with the present specification is defined broadly as a protein that binds specifically to an epitope or antigenic determinant of a polypeptide of the invention.

This invention provides a monoclonal antibody that binds specifically to an antigenic determinant in the amino acid sequence of SEQ ID NO: 1 or a mutant form thereof. In one embodiment, the antigenic determinant to which the monoclonal antibody binds is located within a protein domain responsible for the protein's transporter functions.

As described above, the antibodies are preferably monoclonal, but may also be polyclonal. Monoclonal antibodies may be produced by methods known in the art. These methods include the immunological method described by Kohler and Millstein in Nature vol. 256, pp 495-497 (1975) and by Campbell in “Monoclonal Antibody Technology, The Production And Characterization Of Rodent And Human Hybridomas,” in Burdon, et al. (Eds.), Laboratory Techniques in Biochemistry and Molecular Biology, vol. 13, Elsebier Science Publishers, Amsterdam, Nebr. (1985); and Coligan, J. E., et al. (Eds.), Current Protocols in Immunology, Wiley Intersciences, Inc., New York, (1999); as well as the recombinant DNA method described by Huse, et al., Science, 246:1275-1281 (1989). The recombinant DNA method preferably comprises screening phage libraries for human antibody fragments.

In order to produce monoclonal antibodies, a host mammal is inoculated with a HNTTBMY1 peptide or peptide fragment as described above, and then boosted. Spleens are collected from inoculated mammals a few days after the final boost. Cell suspensions from the spleens are fused with a target cell in accordance with the general method described by Kohler and Millstein, Nature, 256:495-497 (1975). In order to be useful, the peptide fragment must contain sufficient amino acid residues to define the epitope of the molecule being detected.

If using a fragment that is too short to be immunogenic, it may be conjugated to a carrier molecular. Some suitable carrier molecules include key hold limpet, hemocyanin and bovine serum albumin. Conjugation may be carried out by methods known in the art. See Coligan, J. E., et al. (Eds.), Current Protocols in Immunology, Chapter 9, Wiley Intersciences, N.Y. (1999). One such method is to combine a cysteine residue of the fragment with a cysteine residue on the carrier molecule.

### Method of Use Diagnostic Assays

The present invention provides a method of determining if a patient is at risk for a disorder or has a disorder associated with aberrant expression or activity of a polypeptide or polynucleotide of the invention. The disorders of interest include those related to sodium ion neurotransmitter transport, such as: affective disorders, psychotic, neurological metabolic, and cardiovascular disorders, immune-related disorders, acute heart failure, hypotension, hypertension, endocrinal diseases, growth disorders, neuropathic pain, obesity, anorexia, cancers, bulimia, asthma, Parkinson's disease, dementias, osteoporosis, angina pectoris, and myocardial infarction.

The present invention is also useful in diagnosing behavioral disorders; memory disorders; cognitive disorders; disorders associated with aberrant serotonin expression and/or activity; anxiety, fear, depression, sleep, pain, disorders associated with aberrant maintenance of an attentive or alert state; attention deficit disorders; disorders affecting the ‘reward center’ of the brain; disorders affecting the synthesis, and/or effecting the release of neurotransmitters such as dopamine, opioid peptides, serotonin, GABA, and glutamate; addictive disorders; homeostatic disorders; neuroendocrine disorders; disorders affecting the establishment of long term potentiation; circadian rhythm disorders; disorders associated with the establishment of aberrant sleep/wake cycles; dopaminergic functional disorders; neuronal transmission system disorders, and pain.

The method includes detecting in a patient's specimen the presence or absence of a lesion characterized by an alteration in sequence, expression, post-translational modification, or some combination thereof of a human neurotransmitter transporter comprising the amino acid sequence of SEQ ID NO: 1 or a nucleic acid form comprising the nucleotide sequence of SEQ ID NO: 2 or SEQ ID NO: 3. In one aspect of the invention, the presence or absence of a lesion is characterized by: (a) a mutation of the gene encoding the polypeptide of SEQ ID NO: 1 or a homologue thereof; (b) a mis-expression of the gene; (c) an altered neurotransmitter transporter activity of the polypeptide of SEQ ID NO: 1; (d) a polymorphism in the 5′ untranslated region of SEQ ID NO: 3, and combinations thereof.

A method for employing a probe/primer in a polymerase chain reaction (PCR) is disclosed in U.S. Pat. No. 4,683,195. A suitable hybridization technique can include the steps of: collecting samples from a patient, isolating nucleic acid from the cell sample, contacting the nucleic acid sample with one or more primers that specifically hybridize to the selected nucleotide sequence under conditions that hybridize and amplify the sequence, detecting the presence or absence of an amplification product, or detecting the size of the amplification product, and comparing the length to a control sample. PCR can be used as a preliminary amplification step in conjunction with any of the techniques used for detecting the lesion in a nucleic acid sample.

The invention provides a method for detecting a human neurotransmitter transporter comprising the amino acid sequence of SEQ ID NO: 1 or a fragment or mutant form thereof comprising performing an immunoassay on a biological sample. For example, antibodies which specifically bind HNTTBMY1 proteins may be used for diagnosis of conditions or diseases characterized by mal-expression or modification of a HNTTBMY1 protein. Alternatively, such antibodies may be used in assays to monitor progress of patients being treated with a functional HNTTBMY1 protein or its analogs, agonists, antagonists or inhibitors.

The antibodies used for diagnostic purposes may be prepared in the same manner as those described above. Diagnostic assays for HNTTBMY1 protein include methods that use a labeled antibody to detect HNTTBMY1 protein in human body fluids or extracts of cells or tissues. The antibodies may be used with or without modification, and may be labeled by joining them with a reporter molecule. Such reporter molecules are known in the art.

Various protocols including enzyme linked immunosorbent assay (ELISA) and FACS for measuring HNTTBMY1 protein levels are known in the art. These protocols provide a basis for diagnosing altered or abnormal levels of HNTTBMY1 expression. Normal or standard values for HNTTBMY1 expression may be established by combining body fluids or cell extracts from normal human subjects with antibodies to HNTTBMY1 protein under conditions suitable to form a complex.

Furthermore, functional assays can be used in the diagnosis of a particular disorder. For example, it is known that HNTTBMY1 is a protein associated with transport of neurotransmitters across membranes. Thus, an assay to assess the ability of a particular HNTTBMY1 protein form to achieve neurotransmitter transport can be used in the determination of whether a patient has a particular disorder.

A wide variety of labels and conjugation techniques are known by those skilled in the art and may be used, for example, in the various nucleic acid hybridization assays and amino acid assays. Methods for producing labeled hybridization or PCR probes, for detecting sequences related to polynucleotides encoding HNTTBMY1, include oligo-labeling, mixed translation, and labeling or PCR amplification using a labeled nucleotide, are known in the art.

Suitable labeled reporter molecules which may be used include radionucleotides, enzymes, fluorescents, chemiluminescent, or chromogenic agents as well as substrates, cofactors, inhibitors, magnetic particles, and the like.

In one aspect of the invention, DNA sequences of the HNTTBMY1 gene, which have been amplified by use of PCR, may be screened using mutation-specific probes. For example, these probes are nucleic acid oligomers, each of which contains a region of a HNTTBMY1 gene sequence harboring a known mutation. By use of a battery of such mutation-specific probes, PCR amplification products can be screened to identify the presence of a previously identified mutation in the HNTTBMY1 gene. Such hybridization of mutation-specific probes with amplified HNTTBMY1 nucleic acid sequences can be performed, for example, on a nylon filter. Hybridization to a particular probe under stringent hybridization conditions would indicate the presence of the same mutation in the diseased tissue or tissue sample as in the mutation-specific probe. In a related aspect, hybridization with PCR probes which are specific for the wild-type sequence, may be used to identify mutations in the wild-type sequence. The ability of the probe to identify naturally occurring sequences encoding HNTTBMY1, mutations, or related sequences, will depend on the stringency of the hybridization or amplification.

Probes used for the detection of HNTTBMY1-related sequences should preferably contain at least 50% of the nucleotides from any of the HNTTBMY1 encoding sequences. The hybridization probes of the invention may be DNA or RNA and are preferably derived from the nucleotide sequence of SEQ ID NO: 2 corresponding to cDNA encoding human HNTTBMY1 protein or from a genomic sequence including promoter, enhancer elements, and introns of the naturally occurring HNTTBMY1 nucleic acid form. The hybridization probes may be derived from the non-coding sequence 5′ of the coding sequence for the neurotransmitter transporter of the present invention. This 5′ untranslated region is shown in SEQ ID NO: 3.

Additional diagnostic uses for oligonucleotides designed from the sequences encoding HNTTBMY1 protein may involve the use of PCR. Such oligomers may be chemically synthesized by methods described above. Moreover, such oligonucleotides may be generated enzymatically or produced from a recombinant source. Oligomers will preferably consist of two nucleotide sequences, one with sense orientation and another with anti-sense orientation, employed under optimized conditions for identification of a specific gene or condition. The specific oligomers or, alternatively, a degenerate pool of oligomers, may be used under less stringent conditions for the detection and/or quantitation of closely related DNA or RNA sequences.

In a particular aspect, primer pairs of the present invention are useful for determination of the nucleotide sequence of a particular mutated form of HNTTBMY1 gene using PCR. The pairs of single-stranded DNA primers can be annealed to sequences within or surrounding the HNTTBMY1 gene in order to amplify DNA sequence of the HNTTBMY1 gene itself. A complete set of these primers allows synthesis of all of the nucleotides of the HNTTBMY1 gene coding sequences, that is, the exons. The set of primers preferably allows synthesis of both intron and exon sequences. Mutation-specific primers can also be used, wherein such primers anneal only to particular HNTTBMY1 mutant genes, and thus will only amplify a product in the presence of the mutant gene as a template.

Alteration of HNTTBMY1 mRNA expressions can be detected by any techniques known in the art. These include Northern blot analysis, PCR amplification, as well as Rnase protection. Diminished mRNA expression indicates an alteration of the wild-type HNTTBMY1 gene.

Alteration of the wild-type HNTTBMY1 gene can also be detected by screening for alteration of wild-type HNTTBMY1 protein. For example, as described above, antibodies such as monoclonal antibodies immunoreactive with HNTTBMY1 can be used to screen a tissue. Lack of cognate antigen would indicate a HNTTBMY1 mutation. Antibodies specific for products of mutant HNTTBMY1 genes could also be used to detect a mutant HNTTBMY1 gene product. Such immunological assays can be done in any convenient format known in the art. These include Western blot, immunoblots, chemical assays and ELISA (Enzyme Linked Antibody Assay). See Kenneth et al., Monoclonal Antibodies, Plenum Press, New York (1981).

Polypeptide or polynucleotides and/or agonist or antagonists of the present invention may also be used to prepare individuals for extraterrestrial travel, low gravity environments, prolonged exposure to extraterrestrial radiation levels, low oxygen levels, reduction of metabolic activity, exposure to extraterrestrial pathogens, etc. Such a use may be administered either prior to an extraterrestrial event, during an extraterrestrial event, or both. Moreover, such a use may result in a number of beneficial changes in the recipient, such as, for example, any one of the following, non-limiting, effects: an increased level of hematopoietic cells, particularly red blood cells which would aid the recipient in coping with low oxygen levels; an increased level of B-cells, T-cells, antigen presenting cells, and/or macrophages, which would aid the recipient in coping with exposure to extraterrestrial pathogens, for example; a temporary (i.e., reversible) inhibition of hematopoietic cell production which would aid the recipient in coping with exposure to extraterrestrial radiation levels; increase and/or stability of bone mass which would aid the recipient in coping with low gravity environments; and/or decreased metabolism which would effectively facilitate the recipients ability to prolong their extraterrestrial travel by any one of the following, non-limiting means: (i) aid the recipient by decreasing their basal daily energy requirements; (ii) effectively lower the level of oxidative and/or metabolic stress in recipient (i.e., to enable recipient to cope with increased extraterrestial radiation levels by decreasing the level of internal oxidative/metabolic damage acquired during normal basal energy requirements; and/or (iii) enabling recipient to subsist at a lower metabolic temperature (i.e., cryogenic, and/or sub-cryogenic environment).

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein.).

### Method of Use: Identifying Therapeutic Agents and Substrates

The present invention provides a method for identifying therapeutic agents that inhibit, potentiate, or mimic the ability of a polypeptide of the invention to modulate the transport of neurotransmitters. The method includes: treating a cell with an effective amount of at least one candidate so as to alter a transmitter transporter activity associated with the polypeptide of SEQ ID NO: 1, or a fragment or a mutant form thereof, and (b) measuring the effect of the candidate on the cell. In one aspect, candidates which are therapeutic agents that modulate or mediate the cellular function described above, do so by an amount of at least 10% relative to the cell in the absence of the candidates. Preferably, the cell is one of brain or spinal cord. In one embodiment, the method includes: treating a cell with an effective amount of at least one candidate so as to alter regulation of gene expression; and measuring the effect of the candidate on the cell.

In one aspect of the invention, a polypeptide of the invention having SEQ ID NO: 1, its catalytic or immunogenic fragments, mutant fragments or forms, or oligopeptides thereof, can be used for screening libraries of compounds using any of a variety of drug screening techniques. The polypeptide employed in such a screening may be free in solution, affixed to a solid support, borne on a cell surface, or located intracellularly. The formation of binding complexes between an inventive polypeptide and the agent being tested, may be measured.

One technique for screening therapeutic agents which may be used is described in PCT Application WO 84/03564. Briefly, in this method, as it is applied to HNTTBMY1, large numbers of different small test compounds are synthesized on a solid support. The test compounds are reacted with HNTTBMY1 polypeptide, or a fragment thereof, and washed. A binding assay detects bound HNTTBMY1 polypeptide according to methods well known in the art. Alternatively, non-neutralizing antibody test compounds can be used to capture the inventive polypeptide and immobilize it on a solid support.

In another embodiment, one may use competitive therapeutic agent screening assays in which neutralizing antibodies capable of binding HNTTBMY1 protein specifically compete with a test compound for binding HNTFBMY1 protein.

A binding assay may involve a single-step assay. For example, in a one-step assay, the target molecule, which in this case would be the HNTTBMY1 protein, is immobilized and incubated with a labeled drug candidate. The labeled drug candidate binds to the immobilized protein molecule. After washing to remove unbound molecules, the sample is assayed for the presence of the label to determine if binding has occurred and the extent to which it has occurred. Immobilization of the protein may be accomplished in the aforementioned binding assay by immobilizing it onto a solid phase, such as a chromatography column. Such immobilization techniques are well known in the art. For example, the immobilized protein may be covalently or physically bound to the solid phase support, by techniques such as covalent bonding via an amide or ester linkage or by absorption.

In the binding assay described above, the immobilized protein and labeled drug are incubated under conditions and for a period of time sufficient to allow the drug candidate to bind to the immobilized protein. In general, it is desirable to provide incubation conditions sufficient to bind as much of the protein as possible, since this maximizes the binding of the labeled drug to the solid phase, thereby increasing the signal. The specific concentrations of the labeled drug and immobilized protein, the temperature and time of incubation, as well as other such assay conditions, can be varied, depending upon various factors including the concentration of the protein and the sample, the nature of the sample and the like.

Those skilled in the art will be able to determine operative and optimal assay conditions for each determination by employing routine experimentation. The label may be radioactive. Some examples of useful radioactive labels include ³²P, ¹²⁵I, ¹³¹I, ³⁵S, ¹⁴C, and ³H. Use of radioactive labels have been described in U.K. 2,034, 323, U.S. Pat. No. 4,358,535, and U.S. Pat. No. 4,302,204. Some examples of non-radioactive labels include enzymes and chromofores.

Some useful enzymatic labels include enzymes that cause a detectable change in a substrate. Some useful enzymes and/or substrate include for example horseradish peroxidase (pyrogallol and o-phenylenediamine), beta-galatosidase (fluorescein beta-D-galacopyranoside)—and alkaline phosphatase (5-bromo-4-chloro-3-indolyl phosphate-nitroblue tetrazolium). The use of enzymatic labels have been described in U.K. 2,019,404, EP 63,879, Ausubel, F. M. et al.. (Eds.), Current Protocols in Molecular Biology, John Wiley & Sons, Inc., New York (1999), and by Rotman, Proc. Natl. Acad. Sci. USA 47:1981-1991 (1961).

Useful chromofores include, for example, fluorescent, chemiluminescent and bioluminescent molecules, as well a dyes. Some specific chromofores useful in the present method of this invention include, for example, fluorescein, rhodamine, Texas red, phycoerythrin, umbelliferone, and luminol.

The labels may be conjugated to the drug candidate by methods that are well known in the art. The labels may be directly attached through a functional group the drug candidate. The drug candidate may contain or can be caused to contain a functional group. Some examples of suitable functional groups include for example, amino, carboxyl, sulfhydryls, maleimide, isocyanate, isothiocyanate. The aforementioned methods provide for high through-put screening of compounds having suitable binding affinity to the HNTTBMY1 protein of interest. Such compounds provide a means for modulating its activity.

This invention further provides a method for determining whether a substrate not known to be capable of binding to the inventive transporter represented by SEQ ID NO: 1 can bind to this transporter. The method includes the steps of contacting a mammalian cell that includes a DNA molecule encoding the inventive transporter with the substrate under conditions permitting binding of substrates known to bind to transporters, detecting the presence of any of the substrate bound to the inventive transporter, and thereby determining whether the substrate binds to the inventive transporter. The DNA in the cell may have a coding sequence substantially the same as the coding sequence shown in SEQ ID NO: 2. Preferably, the cell is non-neuronal in origin. One example of a non-neuronal cell is a Cos7 cell. In one preferred method for determining whether a substrate is capable of binding to the mammalian transporter, one contacts a non-neuronal cell which has been transfected with the transporter with the substrate under conditions which are known to prevail, or to be associated with, in vivo binding of the substrate to a transporter. Alternatively, a membrane preparation which has been derived from a transfected cell may be contacted with the substrate under these conditions. One detects the presence of any of the substrate being tested bound to the transporter on the surface of the cell, and thereby determines whether the substrate binds to the transporter. In particular, this kind of response system is obtained by transfection of isolated DNA (such as the cDNA sequence shown in SEQ ID NO: 2) into a suitable host cell. Transfection systems are useful as living cell cultures for competitive binding assays between known or candidate drugs and substrates which bind to transporters and which are labeled by radioactive, spectroscopic or other reagents. Membrane preparations containing the inventive transporter isolated from transfected cells are also useful for these competitive binding assays. A transfection system constitutes a drug discovery system useful herein for the identification of either natural or synthetic compounds with potential for drug development that can be further modified or used directly as therapeutic compounds in order to activate or inhibit the natural functions of the inventive transporter. The transfection system is also useful for determining the affinity and efficacy of known drugs at the inventive transporter sites.

The human HNTTBMY1 polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a HNTTBMY1 polypeptide, or a bindable peptide fragment, of this invention, comprising providing a plurality of compounds, combining the HNTTBMY1 polypeptide, or a bindable peptide fragment, with each of a plurality of compounds for a time sufficient to allow binding under suitable conditions and detecting binding of the HNTTBMY1 polypeptide or peptide to each of the plurality of test compounds, thereby identifying the compounds that specifically bind to the HNTTBMY1 polypeptide or peptide.

Methods of identifying compounds that modulate the activity of the novel human HNTTBMY1 polypeptides and/or peptides are provided by the present invention and comprise combining a potential or candidate compound or drug modulator of neurotransmitter transporter biological activity with an HNTTBMY1 polypeptide or peptide, for example, the HNTTBMY1 amino acid sequence as set forth in SEQ ID NO: 2, and measuring an effect of the candidate compound or drug modulator on the biological activity of the HNTTBMY1 polypeptide or peptide. Such measurable effects include, for example, physical binding interaction; the ability to cleave a suitable neurotransmitter transporter substrate; effects on native and cloned HNTTBMY1-expressing cell line; and effects of modulators or other neurotransmitter transporter-mediated physiological measures.

Another method of identifying compounds that modulate the biological activity of the novel HNTTBMY1 polypeptides of the present invention comprises combining a potential or candidate compound or drug modulator of a neurotransmitter transporter biological activity with a host cell that expresses the HNTTBMY1 polypeptide and measuring an effect of the candidate compound or drug modulator on the biological activity of the HNTTBMY1 polypeptide. The host cell can also be capable of being induced to express the HNTTBMY1 polypeptide, e.g. via inducible expression. Physiological effects of a given modulator candidate on the HNTTBMY1 polypeptide can also be measured. Thus, cellular assays for particular neurotransmitter transporter modulators may be either direct measurement or quantification of the physical biological activity of the HNTTBMY1 polypeptide, or they may be measurement or quantification of a physiological effect. Such methods preferably employ a HNTTBMY1 polypeptide as described herein, or an overexpressed recombinant HNTTBMY1 polypeptide in suitable host cells containing an expression vector as described herein, wherein the HNTTBMY1 polypeptide is expressed, overexpressed, or undergoes upregulated expression.

Another aspect of the present invention embraces a method of screening for a compound that is capable of modulating the biological activity of a HNTTBMY1 polypeptide, comprising providing a host cell containing an expression vector harboring a nucleic acid sequence encoding a HNTTBMY1 polypeptide, or a functional peptide or portion thereof (e.g., SEQ ID NOS: 2); determining the biological activity of the expressed HNTTBMY1 polypeptide in the absence of a modulator compound; contacting the cell with the modulator compound and determining the biological activity of the expressed HNTTBMY1 polypeptide in the presence of the modulator compound. In such a method, a difference between the activity of the HNTTBMY1 polypeptide in the presence of the modulator compound and in the absence of the modulator compound indicates a modulating effect of the compound.

Essentially any chemical compound can be employed as a potential modulator or ligand in the assays according to the present invention. Compounds tested as neurotransmitter transporter modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel HNTTBMY1 polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptides (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a HNTTBMY1 polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a HNTTBMY1 polypeptide or peptide to measure a biological binding or ligand binding activity, the source may be a whole cell lysate that can be prepared by successive freeze-thaw cycles (e.g., one to three) in the presence of standard protease inhibitors. The HNTTBMY1 polypeptide may be partially or completely purified by standard protein purification methods, e.g., affinity chromatography using specific antibody described infra, or by ligands specific for an epitope tag engineered into the recombinant HNTTBMY1 polypeptide molecule, also as described herein. Binding activity can then be measured as described.

Compounds which are identified according to the methods provided herein, and which modulate or regulate the biological activity or physiology of the HNTTBMY1 polypeptides according to the present invention are a preferred embodiment of this invention. It is contemplated that such modulatory compounds may be employed in treatment and therapeutic methods for treating a condition that is mediated by the novel HNTTBMY1 polypeptides by administering to an individual in need of such treatment a therapeutically effective amount of the compound identified by the methods described herein.

In addition, the present invention provides methods for treating an individual in need of such treatment for a disease, disorder, or condition that is mediated by the HNTTBMY1 polypeptides of the invention, comprising administering to the individual a therapeutically effective amount of the HNTTBMY1-modulating compound identified by a method provided herein.

### EXAMPLES

### Example 1

Bioinformatics Analysis

A. In Silco Identification of Express Sequence Tag in Brain Amygdala Region

The first approach used to identify the gene encoding the orphan neurotransmitter transporter of the present invention was to search commercial gene sequence libraries reporting Express Sequence Tags (EST's) from Incyte Genomics, Inc. (Palo Alto, Calif.). The EST's represent short stretches (ranging from about 250 bp to about 450 bp) of sequenced cDNA's (from the 5′ and 3′ end) that are synthesized from isolated messenger RNAs (mRNAs) which encode human proteins. Incyte's EST libraries were searched for genes specific to Amygdala, a brain sub-region involved in affective disorders. The identified ESTs were then searched against the non-redundant protein and patent databases (www.ncbi.nlm.nih.gov). An EST not represented in these public databases was identified (clone ID 8229031) and the corresponding EST contig was obtained from Incyte (Incyte ContigID: 181961). EST contigs are consensus sequences created from EST sequences using the LifeSeq® gene-by-gene assembly algorithm.

B. Development of a Predicted Full Length Sequence

The human genomic contig (GenBank Accession: NT_{—}004698) sequence was aligned with the EST contig, an orphan neurotransmitter transporter protein sequence (NTF4 from Rat, SEQ ID NO: 13) and Hidden Markov Model using Genewise (http://www.sanger.ac.uk./Software/Wise2/) to derive a predicted full length sequence of clone HNTTBMY1, corresponding to the human orphan neurotransmitter transporter of the present invention. Genewise is a program that compares the genomic sequence to protein reference sequences and/or to Hidden Markov Models (HMM's) representing protein domains. The comparison is performed at the protein translation level, while simultaneously maintaining a reading frame regardless of intervening introns and sequencing errors, which may otherwise cause frame shifts. Thus, a combination of:

(a) the EST (expressed sequence tag) contig information,

(b) the Genbank human contig (Accession number NT_{—}004698) and,

(c) the protein match

were used to obtain the predicted full length sequence of the HNTTBMY1 clone corresponding to the human orphan neurotransmitter transporter of the present invention.

C. Classification of the Predicted Full Length Sequence

The complete predicted protein sequence of the inventive transporter (SEQ ID NO: 1), translated from the cDNA sequence of the HNTTBMY1 clone was analyzed for potential transmembrane domains using TMPRED, a program that predicts membrane spanning regions and their orientation, based on the statistical analysis of TMbase, which is a database of naturally occurring TM proteins [Hofmann K., and Stoffel, W., “TMbase—A Database of Membrane Spanning Protein Segments,” Biol. Chem. Hoppe-Seyler, 347:166, (1993)]. TMPRED was thus used to predict the occurrence of transmembrane domains. As shown in FIG. 2, the inventive transporter is predicted to have twelve transmembrane domains, a common feature of Na⁺/Cl⁻ dependent NTTS.

The inventive polypeptide represented by SEQ ID NO: 1 was searched against profile Hidden Markov Models (HMM's) of neurotransmitter transporters generated by Pfam, a database of multiple alignments of protein domains or conserved protein regions (Pfam version 6.6, available at: http://www.pfam.wustl.edu). The alignments represent certain evolutionarily conserved structures, having implications for the protein's function. Profile HMMs are built from the Pfam alignments and can be very useful for automatically recognizing that a new protein belongs to an existing protein family, even if the homology is weak [A. Bateman et al., The Pfam Protein Families Database. Nucleic Acids Research, 28:263-266 (2000)]. Using this analysis, SEQ ID NO: 1 matched significantly to the transmembrane sodium symporter family Pfam model (FIG. 1). The orphan protein of the present invention is predicted to be a novel 12 transmembrane domain orphan human Na⁺/Cl⁻ dependent NTT based on sequence, structure, TMPRED analysis, and significant match to NTT Pfam domains.

The inventive polypeptide of SEQ ID NO: 1 was aligned with other related neurotransmitter transporter sequences as shown in FIG. 3 and Table I. The alignment was carried out using the PILEUP program in GCG version 10.2. As shown in Table I below the inventive human transporter shows approximately 96.8% sequence identity with NTT4 from rat (also referred to as Rxt1 and rat xt1) and approximately 96.0% sequence identity with bovine NTT4.

The NTT4 rat transporter has been classified as a membrane-bound orphan Na⁺/Cl⁻ dependent transporter. It has been reported that this transporter is widely distributed in brain with the highest levels of expression in cerebral cortex and thalamus, [Luque, J. M., et al. European J Neurosci., 8(1):127-137, (1996)], where it is associated with synaptic vesicles in nerve terminals of glutamatergic neurons and of some GABAergic neurons [C. Cireli and G. Tononi, Brain Research 885, 303-321 (2000)]. As a result of the high homology to the rat and bovine NTT4 transporters, the inventors have concluded that the inventive cDNA sequence of SEQ ID NO: 2 resulted from an identical gene of the human genome.

D. Selection of Probes and Primers for Use in cDNA Cloning

Nucleotide sequences predicted for the HNTTBMY1 clone which were derived from the bioinformatics analysis described above are used to develop gene-specific PCR primers and cloning oligoprobes, as follows:

(a) PCR Gene Specific Primer (GSP) pairs that reside within a single predicted exon

(b) PCR primers that cross putative exon/intron boundaries; and

(c) 80 mer antisense and sense oligos containing a biotin moiety on the 5′ end.

The information obtained from the b group primers is used to assess which putative expressed sequences can be experimentally observed to have reverse transcriptase dependent expression. The primer pairs from the a group are less stringent in terms of identifying expressed sequences. However, since they amplify genomic DNA as well as cDNA, their ability to amplify genomic DNA allows for the necessary positive control for the primer pair. Negative results with the b group are subjected to the caveat that the sequence may not be expressed in the tissue first strand that is under examination. The below Table shows the location of the expected hybridization sites of exemplary primers/probes.

Expected Hybridization Sites for Selected Primers and Probes

| Primer/Probe | Binding Site | SEQ ID NO. |
|---|---|---|
| GSP Primer 1 Forward | 1978-1997 | 10 |
| GSP Primer 1 Reverse | 2213-2232 | 11 |
| GSP Primer 2 Forward | 1563-1582 | 9 |
| GSP Primer 2 Reverse | 1670-1689 | 8 |
| Oligo 2 (80 mer) | 1588-1667 | 5 |
| Oligo 1 (80 mer) | 2001-2080 | 4 |

### Example 2

Method for the Construction of a Size Fractionated cDNA Library for the Isolation of Large Insert Clones

Polyadenylated [poly(A)+] RNA from Human amygdala is purchased from Clontech (Palo Alto, Calif.). The Clonetech poly(A)+RNA is subsequently treated with Dnase I to remove traces of genomic DNA contamination and then converted into double stranded cDNA using the SuperScript™ Plasmid System for cDNA Synthesis and Plasmid Cloning (Invitrogen, Carlsbad, Calif.) according to manufacturer's instructions, except that no radioisotope is incorporated in either of the cDNA synthesis steps.

The cDNA is then size fractionated on a TransGenomics HPLC system equipped with a size exclusion column from TosoHass (Stuttgart, Germany) with dimensions of 7.8 mm×30 cm and a particle size of 10 (m. Tris buffered saline is used as the mobile phase, and the column is run at a flow rate of 0.5 mL/min. The system is calibrated by running a 1 kb ladder through the column and analyzing the fractions by agarose gel electrophoresis. Using these data, it can be determine which fractions are to be pooled to obtain the largest cDNA library. Generally, fractions that eluted in the range of 12 to 15 minutes are used.

After selection, the cDNA is precipitated, concentrated, and then ligated into the Sal I /Not I sites in a pSPORT vector (e.g. pCMVSPORT, pSPORT and pSPORT2) The vectors are then electroporated into DH12S cells (Invitrogen, Carlsbad, Calif.) in order to transform the cells.

### Example 3

Conversion of Double Stranded cDNA Libraries into Single Strand Circular Form

pSPORT vectors contain an f1 intergenic region which may be used for the production of single stranded DNA. In this procedure, the vectors containing cDNA inserts are introduced into a strain of E. coli containing the F′ episome, such as DH12S (Invitrogen, Carlsbad, Calif.), and these cells are then infected with a helper phage such as M13K07. The plasmids are then rescued as single-stranded circles that are secreted into a growth medium as phage-like particles.

Specifically, between 200 μL and 1 ml of thawed cDNA library is used to inoculate 200 mL of Luria broth containing 400 μL carb(?). The culture is incubated at 37° C. for 45 minutes with shaking. When the OD 600 reading is between 0.025 to 0.400, 1 mL of M13K07 helper phage is added to the culture. After 2 hours of growth, 500 μL Kanamycin (30 mg/mL) is additionally added and the culture allowed to grow for 15 to 18 more hours. The cells are pelleted by dividing the culture into six 50 mL autoclaved screw-cap tubes and centrifuging at 10, 000 RPMs in an HB-6 rotor for 15 minutes at 4° C. The phage like particles in the supernatant are saved and the pelleted cells are killed with iodine and then discarded. The supernatant is filtered through a 0.2 (m filter and 12000 units of DNAse I is added to the supernatant and allowed to incubate for 90 minutes.

50 mL of ice-cold 40% Polyethylene glycol 8000 and 2.5 M NaCl and 10 mM of MgSO₄ is added to the supernatant to precipitate the phage-like particles. The supernatant is aliquoted into 6 centrifuge tubes, covered with parafilm and then incubated on ice overnight. Phage is subsequently pelleted by centrifugation at 10,000 RPMs in an HB-6 rotor for 20 minutes at 4° C. The supernatant is discarded and care is taken to remove all supernatant by wiping the sides of the centrifuge tubes with kimwipes. Pellets are resuspended in 1 mL of 10 mM Tris-HCl, pH 8.0, 1 mM EDTA, pH 8.0 (TE). The pellets are pooled into a 14 mL Sarstadt tube (6 mL total) and 60 uL freshly made Proteinase K (20 mg/mL) and 1% Sodium-dodecyl sulfate (SDS) (60 uL of stock 10% SDS) are added. The mixture is allowed to incubate at 42° C. for 1 hour.

The proteins from the viral coat are removed using a phenol/chloroform extraction. 1 mL of 5 M NaCl is added to pooled resuspended pellets with an equal volume of phenol chloroform. The resuspension is vortexed, and centrifuged at 5,000 RPMs for 5 minutes at 4° C. The aqueous phase is transferred into a new Sarstadt tube and extractions are repeated until no interface is visible. The DNA is ethanol precipitated, and oligosaccharides are removed by resupending the DNA pellet in 50 μL TE, pH 8, freeze drying on ice for 10 minutes and then centrifuging at 14,000 RPMs for 15 minutes at 4° C. The concentration of DNA is determined by OD readings at 260/280.

### Example 4

Testing Quality of Single Stranded DNA

An aliquot of the single stranded DNA is repaired and tested for quality by ascertaining transformation efficiency.

A DNA reaction mixture is prepared using 1 μL of a 5 ng/(L single stranded DNA solution, 11 (L dH₂O), 1.5 μL 10 (M T7 primer and 1.5 (L 10X Precision-Taq buffer. The repair mix is prepared using 4 uL 5 mM dNTPs (1.25 mM each), 1.5 μL 10X Precision-Taq buffer, 9.25 (L dH₂O, and 0.25 (L Precision-Taq polymerase. This repair mix is preheated to 70(C. and added during the middle of the PCR cycle. Only 1 cycle of PCR is run. The program is as follows: 95(C., 20 sec, 59(C., 1 min; (15 uL repair mix is added at this step) and 73(C., 23 min. The DNA generated from this PCR reaction is ethanol precipitated before electroporation into DH12S cells.

Two microliters of repaired DNA, (about 1.0×10⁻³ μg prepared as described above) is aliquoted into an eppendorf tube. One microliter of a 1 ng/μl unrepaired library is aliquoted into a second tube. One microliter of a 0.01 μg/μL concentration of pUC19 is also aliquoted into a third eppendorf tube to be used as a positive control. DH12S cells are added to pre-chilled cuvettes and allowed to thaw on ice. Both the DNA and cells remain on ice until use. Forty microliters of cells are added to each DNA aliquot. The mixture is pipetted once and the put into a cuvette between metal plates. Electroporation is carrried out at 1.8 kV. Gibco BRL Cell-Porator or similar system is used. 1 ml of SOC media is immediately added (2 g tryptone, 0.5 g yeast extract, 0.25 ml of 1 M KCL, 1 ml of 1 M NaCl, 1 M MgCl₂, 1 M MgSO₄, 1 ml of 2 M glucose to a final volume of 100 mls) to each cuvette. The cells are allowed to recover for 1 hour at 37(C. with shaking (225 rpm).

The cells are then diluted as follows: The repaired DNA is diluted at concentration of 1:100, 1:1000 and 1:10,000. The unrepaired library DNA is diluted at concentrations of 1:10 and 1:100, pUC19 is diluted at concentrations of 1:10 and 1:100. The cells are plated on LB and carbenicillin plates (100 μl of each dilution). The plates are allowed to incubate overnight at 37(C. Colonies are counted for each plate. The plate for each test (repaired, unrepaired and pUC) which contains the lowest countable dilution is used to calculate the titer using the following equation:

(Number of colonies)(dilution factor)( )(200 uL/100 uL)(1000 uL/20 uL)=CFUs(colony forming units)

The CFUs/mg of DNA used is calculated and the percent of background is ascertained by the following equation:

% Background=(unrepaired CFU/μg/repaired CFU/μg)×100%

### Example 5

Selection of a Primary Selected Library

One microliter of anti-sense biotinylated probes (or sense oligos when annealing to single stranded DNA from pSPORT2 vector) containing one hundred and fifty nanograms of 1 to 50 different 80 mer oligo probes is added to six microliters (six micrograms) of a mixture of up to 15 single-stranded covalently closed circular cDNA libraries and seven microliters of 100% formamide in a 0.5 ml PCR tube. SEQ ID NO: 4 and/or SEQ ID NO: 5 are used as biotinylated probes in this step. The probes are located between bases 1613-1692 and bases 1200-1279, respectively, in SEQ ID NO: 2.

The mixture is heated in a thermal cycler to 95(C. for 2 min. Fourteen microliters of 2× hybridization buffer (50% formamide, 1.5 M NaCl, 0.04 M NaPO₄, pH 7.2, 5 mM EDTA, 0.2% SDS) is added to the heated probe/cDNA library mixture and incubated at 42(C. for 26 hours. Hybrids between the biotinylated oligo and the circular cDNA are isolated by diluting the hybridization mixture to 220 microliters in a solution containing 1 M NaCl, 10 mM Tris-HCl pH 7.5, 1 mM EDTA, pH 8.0 and adding 125 microliters of streptavidin magnetic beads. This solution is incubated at 42(C. for 60 min, and mixed every 5-min to re-suspend the beads. The beads are separated from the solution with a magnet and washed three times in 200 microliters of 0.1× SSPE, 0.1% SDS at 45(C.

The single stranded cDNAs are released from the biotinylated oligo/streptavidin magnetic bead complex by adding 50 microliters of 0.1 N NaOH and incubating at room temperature for 10 min. Six microliters of 3 M sodium acetate is added along with 15 micrograms of glycogen and the solution is ethanol precipitated with 120 microliters of 100% ethanol. The precipitated DNA is re-suspend in 12 microliters of TE (10 mM Reia-Hcl, pH 8.0; 1 mM MEDTA).

Conversion of Single Stranded cDNA to Double Stranded cDNA

The single stranded cDNA is converted into double strands in a thermal cycler by mixing 5 microliters of the captured DNA with 1.5 microliters of 10 micromolar standard SP6 primer (SEQ ID NO: 7) for libraries in pSPORT 1 and pSPORT 2 and T7 primer (SEQ ID NO: 6) for libraries in pCMVSPORT and 1.5 microliters of 10× PCR buffer.

The mixture is heated to 95(C. for 20 seconds then ramped down to 59 (C. At this time 15 microliters of a repair mix (as described above), is preheated to 70(C. and added to the DNA. The solution is ramped back to 73(C. and incubated for 23 min. The repaired DNA is ethanol precipitated and re-suspended in 10 microliters of Tris-EDTA at pH 8.0. Two microliters are electroporated per tube containing 40 microliters of E. coli DH12S cells. Three hundred and thirty three microliters are plated onto one 150-mm plate of LB agar plus 100 micrograms/milliliter of ampicillin with nylon filters. After overnight incubation at 37(C., the colonies from all plates are harvested by scraping into 10 mls of LB+50 micrograms/milliliter of ampicillin and 2 mls of sterile glycerol.

### Example 6

Normalization of Primary Library

The second round of selection is initiated by making single-strand circular DNA from approximately one-fifth of the primary selected library using the method listed above.

A secondary hybridization capture is carried out with 80 mer oligos as described herein for only those sequences that were positive for gene specific primers. After the second round, the captured single strand DNAs are repaired with a pool of GSPs where only the primer complementary to polarity of the single-stranded circular DNA is used (the antisense pirmer for pCMVSPORT and pSPORT1 and the sense primer for pSPORT2). The sequences of the Gene-Specific-Primer (GSP) pairs used to identify the various targeted cDNAs in the primary selected single stranded cDNA libraries are Pair 1 (SEQ ID NO: 10 and SEQ ID NO: 11) and Pair 2 (SEQ ID NO: 9 and SEQ ID NO: 8).

The repaired DNAs are electroporated into DH10B and the resulting colonies inoculated into 96 deep well blocks. After overnight growth, DNA is prepared and sequentially screened for each of the targeted sequences using the GSPs by PCR. Typically, greater than 80% of the clones are positive for any given GSP. Selected clones are sequenced.

### Example 7

Isolation of a Specific Clone from the Deposited Sample

The deposited material in the sample assigned the ATCC Deposit Number cited in Table II for any given cDNA clone also may contain one or more additional plasmids, each comprising a cDNA clone different from that given clone. Thus, deposits sharing the same ATCC Deposit Number contain at least a plasmid for each cDNA clone identified in Table II. Typically, each ATCC deposit sample cited in Table II comprises a mixture of approximately equal amounts (by weight) of about 1-10 plasmid DNAs, each containing a different cDNA clone and/or partial cDNA clone; but such a deposit sample may include plasmids for more or less than 2 cDNA clones.

Two approaches can be used to isolate a particular clone from the deposited sample of plasmid DNA(s) cited for that clone in Table II. First, a plasmid is directly isolated by screening the clones using a polynucleotide probe corresponding to SEQ ID NO: 1.

Particularly, a specific polynucleotide with 30-40 nucleotides is synthesized using an Applied Biosystems DNA synthesizer according to the sequence reported. The oligonucleotide is labeled, for instance, with 32P-(-ATP using T4 polynucleotide kinase and purified according to routine methods. (E.g., Maniatis et al., Molecular Cloning: A Laboratory Manual, Cold Spring Harbor Press, Cold Spring, N.Y. (1982).) The plasmid mixture is transformed into a suitable host, as indicated above (such as XL-1 Blue (Stratagene)) using techniques known to those of skill in the art, such as those provided by the vector supplier or in related publications or patents cited above. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO: 2 (i.e., within the region of SEQ ID NO: 2 bounded by the 5′ NT and the 3′ NT of the clone defined in Table II) are synthesized and used to amplify the desired cDNA using the deposited cDNA plasmid as a template. The polymerase chain reaction is carried out under routine conditions, for instance, in 25 ul of reaction mixture with 0.5 ug of the above cDNA template. A convenient reaction mixture is 1.5-5 mM MgCl2, 0.01% (w/v) gelatin, 20 uM each of dATP, dCTP, dGTP, dTTP, 25 pmol of each primer and 0.25 Unit of Taq polymerase. Thirty five cycles of PCR (denaturation at 94 degree C. for 1 min; annealing at 55 degree C. for 1 min; elongation at 72 degree C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

The polynucleotide(s) of the present invention, the polynucleotide encoding the polypeptide of the present invention, or the polypeptide encoded by the deposited clone may represent partial, or incomplete versions of the complete coding region (i.e., full-length gene). Several methods are known in the art for the identification of the 5′ or 3′ non-coding and/or coding portions of a gene which may not be present in the deposited clone. The methods that follow are exemplary and should not be construed as limiting the scope of the invention. These methods include but are not limited to, filter probing, clone enrichment using specific probes, and protocols similar or identical to 5′ and 3′ “RACE” protocols that are well known in the art. For instance, a method similar to 5′ RACE is available for generating the missing 5′ end of a desired full-length transcript. (Fromont-Racine et al., Nucleic Acids Res. 21(7):1683-1684 (1993)).

Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full-length gene.

This above method starts with total RNA isolated from the desired source, although poly-A+ RNA can be used. The RNA preparation can then be treated with phosphatase if necessary to eliminate 5′ phosphate groups on degraded or damaged RNA that may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene. Moreover, it may be advantageous to optimize the RACE protocol to increase the probability of isolating additional 5′ or 3′ coding or non-coding sequences. Various methods of optimizing a RACE protocol are known in the art, though a detailed description summarizing these methods can be found in B. C. Schaefer, Anal. Biochem., 227:255-273, (1995).

An alternative method for carrying out 5′ or 3′ RACE for the identification of coding or non-coding sequences is provided by Frohman, M. A., et al., Proc. Nat'l. Acad. Sci. USA, 85:8998-9002 (1988). Briefly, a cDNA clone missing either the 5′ or 3′ end can be reconstructed to include the absent base pairs extending to the translational start or stop codon, respectively. In some cases, cDNAs are missing the start of translation, therefor. The following briefly describes a modification of this original 5′ RACE procedure. Poly A+ or total RNAs reverse transcribed with Superscript II (Gibco/BRL) and an antisense or I complementary primer specific to the cDNA sequence. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the dA-tail in PCR buffer, Taq DNA polymerase (Perkin-Elmer Cetus), an oligo-dT primer containing three adjacent restriction sites (XhoIJ Sail and ClaI) at the 5′ end and a primer containing just these restriction sites. This double-stranded cDNA is PCR amplified for 40 cycles with the same primers as well as a nested cDNA-specific antisense primer. The PCR products are size-separated on an ethidium bromide-agarose gel and the region of gel containing cDNA products the predicted size of missing protein-coding DNA is removed. cDNA is purified from the agarose with the Magic PCR Prep kit (Promega), restriction digested with XhoI or SalI, and ligated to a plasmid such as pBluescript SKII (Stratagene) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone. Similar methods known in the art and/or commercial kits are used to amplify and recover 3′ ends.

Several quality-controlled kits are commercially available for purchase. Similar reagents and methods to those above are supplied in kit form from Gibco/BRL for both 5′ and 3′ RACE for recovery of full length genes. A second kit is available from Clontech which is a modification of a related technique, SLIC (single-stranded ligation to single-stranded cDNA), developed by Dumas et al., Nucleic Acids Res., 19:5227-32(1991). The major differences in procedure are that the RNA is alkaline hydrolyzed after reverse transcription and RNA ligase is used to join a restriction site-containing anchor primer to the first-strand cDNA. This obviates the necessity for the dA-tailing reaction which results in a polyT stretch that is difficult to sequence past.

An alternative to generating 5′ or 3′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

RNA Ligase Protocol For Generating The 5′ or 3′ End Sequences To Obtain Full Length Genes

Once a gene of interest is identified, several methods are available for the identification of the 5′ or 3′ portions of the gene which may not be present in the original cDNA plasmid. These methods include, but are not limited to, filter probing, clone enrichment using specific probes and protocols similar and identical to 5′ and 3′ RACE. While the full-length gene may be present in the library and can be identified by probing, a useful method for generating the 5′ or 3′ end is to use the existing sequence information from the original cDNA to generate the missing information. A method similar to 5′ RACE is available for generating the missing 5′ end of a desired full-length gene. (This method was published by Fromont-Racine et al., Nucleic Acids Res., 21(7): 1683-1684 (1993). Briefly, a specific RNA oligonucleotide is ligated to the 5′ ends of a population of RNA presumably 30 containing full-length gene RNA transcript and a primer set containing a primer specific to the ligated RNA oligonucleotide and a primer specific to a known sequence of the gene of interest, is used to PCR amplify the 5′ portion of the desired full length gene which may then be sequenced and used to generate the full length gene. This method starts with total RNA isolated from the desired source, poly A RNA may be used but is not a prerequisite for this procedure. The RNA preparation may then be treated with phosphatase if necessary to eliminate 5′ phosphate groups on degraded or damaged RNA which may interfere with the later RNA ligase step. The phosphatase if used is then inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase. This modified RNA preparation can then be used as a template for first strand cDNA synthesis using a gene specific oligonucleotide. The first strand synthesis reaction can then be used as a template for PCR amplification of the desired 5′ end using a primer specific to the ligated RNA oligonucleotide and a primer specific to the known sequence of the apoptosis related of interest. The resultant product is then sequenced and analyzed to confirm that the 5′ end sequence belongs to the relevant apoptosis related.

### Example 8

Method of Assessing the Expression Profile of the Novel HNTTBMY1 Polypeptides of the Present Invention Using Expanded mRNA Tissue and Cell Sources

Total RNA from tissues was isolated using the TriZol protocol (Invitrogen) and quantified by determining its absorbance at 260 nM. An assessment of the 18 s and 28 s ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For HNTTBMY1, the primer probe sequences were as follows

| | |
|---|---|
| Forward Primer | |
| 5′-CACCCTCTCCGTGTCCTACAA-3′ | (SEQ ID NO:96) |
| Reverse Primer | |
| 5′-TCTCATCGTTCTCCTCCAGGTT-3′ | (SEQ ID NO:97) |
| TaqMan Probe | |
| 5′-AGATGTCCTTCATCATGCGGCCCTT-3′ | (SEQ ID NO:98) |

DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with gene-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+ RNA. If not the RNA was not used in actual experiments.

Reverse Transcription Reaction and Sequence Detection

100 ng of Dnase-treated total RNA was annealed to 2.5 (M of the respective gene-specific reverse primer in the presence of 5.5 mM Magnesium Chloride by heating the sample to 72(C. for 2 min and then cooling to 55(C. for 30 min. 1.25 U/(1 of MuLv reverse transcriptase and 500(M of each dNTP was added to the reaction and the tube was incubated at 37(C. for 30 min. The sample was then heated to 90(C. for 5 min to denature enzyme.

Quantitative sequence detection was carried out on an ABI PRISM 7700 by adding to the reverse transcribed reaction 2.5(M forward and reverse primers, 2.0(M of the TaqMan probe, 500(M of each dNTP, buffer and 5U AmpliTaq Gold. The PCR reaction was then held at 94(C. for 12 min, followed by 40 cycles of 94(C. for 15 sec and 60(C. for 30 sec.

Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{((Ct)}

The expanded expression profile of the HNTTBMY1 polypeptide is provided in FIG. 7 and described elsewhere herein.

### Example 9

Chromosomal Mapping of the Polynucleotides

An oligonucleotide primer set is designed according to the sequence at the 5′ end of SEQ ID NO: 2. This primer preferably spans about 100 nucleotides. This primer set is then used in a polymerase chain reaction under the following set of conditions: 30 seconds,95 degree C.; 1 minute, 56 degree C.; 1 minute, 70 degree C. This cycle is repeated 32 times followed by one 5 minute cycle at 70 degree C. Mammalian DNA, preferably human DNA, is used as template in addition to a somatic cell hybrid panel containing individual chromosomes or chromosome fragments (Bios, Inc). The reactions are analyzed on either 8% polyacrylamide gels or 3.5% agarose gels. Chromosome mapping is determined by the presence of an approximately 100 bp PCR fragment in the particular somatic cell hybrid.

### Example 10

Bacterial Expression of a Polypeptide

A polynucleotide encoding a polypeptide of the present invention is amplified using PCR oligonucleotide primers corresponding to the 5′ and 3′ ends of the DNA sequence, as outlined herein, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BaniHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C or frozen at −80 degree C.

### Example 11

Purification of a Polypeptide From an Inclusion Body

The following alternative method can be used to purify a polypeptide expressed in E coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 12

Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHIl, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined herein, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described herein. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGoldtm baculovirus DNA”, Pharmingen, San Diego, Calif.), using the lipofection method described by Felgner et al., Proc. Natl. Acad. Sci. USA 84:7413-7417 (1987). One ug of BaculoGoldtm virus DNA and 5ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 13

Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDS-PAGE and Western blot or by reversed phase HPLC analysis.

### Example 14

Protein Fusions

The polypeptides of the present invention are preferably fused to other proteins. These fusion proteins can be used for a variety of applications. For example, fusion of the present polypeptides to His-tag, HA-tag, protein A, IgG domains, and maltose binding protein facilitates purification. (See Example described herein; see also EP A 394,827; Traunecker, et al., Nature 331:84-86 (1988).) Similarly, fusion to IgG-1, IgG-3, and albumin increases the half-life time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector. Note that the polynucleotide is cloned without a stop codon, otherwise a fusion protein will not be produced.

The naturally occurring signal sequence may be used to produce the protein (if applicable). Alternatively, if the naturally occurring signal sequence is not used, the vector can be modified to include a heterologous signal sequence. (See, e.g., WO 96/34891 and/or U.S. Pat. No. 6,066,781, supra.)

Human IgG Fc Region:

(SEQ ID NO:56)

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAACC
ACAGGTGTACACCCTGCCCCCATCCCGGGATGAGCTGACCAAGAACCAGG
TCAGCCTGACCTGCCTGGTCAAAGGCTTCTATCCAAGCGACATCGCCGTG
GAGTGGGAGAGCAATGGGCAGCCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGG
TAAATGAGTGCGACGGCCGCGACTCTAGAGGAT

### Example 15

Production of an Antibody From a Polypeptide

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C)., and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### Example 16

Regulation of Protein Expression via Controlled Aggregation in the Endoplasmic Reticulum

As described more particularly herein, proteins regulate diverse cellular processes in higher organisms, ranging from rapid metabolic changes to growth and differentiation. Increased production of specific proteins could be used to prevent certain diseases and/or disease states. Thus, the ability to modulate the expression of specific proteins in an organism would provide significant benefits.

Numerous methods have been developed to date for introducing foreign genes, either under the control of an inducible, constitutively active, or endogenous promoter, into organisms. Of particular interest are the inducible promoters (see, M. Gossen, et al., Proc. Natl. Acad. Sci. USA., 89:5547 (1992); Y. Wang, et al., Proc. Natl. Acad. Sci. USA, 91:8180 (1994), D. No., et al., Proc. Natl. Acad. Sci. USA, 93:3346 (1996); and V. M. Rivera, et al., Nature Med, 2:1028 (1996); in addition to additional examples disclosed elsewhere herein). In one example, the gene for erthropoietin (Epo) was transferred into mice and primates under the control of a small molecule inducer for expression (e.g., tetracycline or rapamycin) (see, D. Bohl, et al., Blood, 92:1512, (1998); K. G. Rendahl, et al., Nat. Biotech, 16:757, (1998); V. M. Rivera, et al., Proc. Natl. Acad. Sci. USA, 96:8657 (1999); and X. Ye et al., Science, 283:88 (1999). Although such systems enable efficient induction of the gene of interest in the organism upon addition of the inducing agent (i.e., tetracycline, rapamycin, etc,.), the levels of expression tend to peak at 24 hours and trail off to background levels after 4 to 14 days. Thus, controlled transient expression is virtually impossible using these systems, though such control would be desirable.

A new alternative method of controlling gene expression levels of a protein from a transgene (i.e., includes stable and transient transformants) has recently been elucidated (V. M. Rivera., et al., Science, 287:826-830, (2000)). This method does not control gene expression at the level of the mRNA like the aforementioned systems. Rather, the system controls the level of protein in an active secreted form. In the absence of the inducing agent, the protein aggregates in the ER and is not secreted. However, addition of the inducing agent results in dis-aggregation of the protein and the subsequent secretion from the ER. Such a system affords low basal secretion, rapid, high level secretion in the presence of the inducing agent, and rapid cessation of secretion upon removal of the inducing agent. In fact, protein secretion reached a maximum level within 30 minutes of induction, and a rapid cessation of secretion within 1 hour of removing the inducing agent. The method is also applicable for controlling the level of production for membrane proteins.

Detailed methods are presented in V. M. Rivera., et al., Science, 287:826-830, (2000)), briefly:

Fusion protein constructs are created using polynucleotide sequences of the present invention with one or more copies (preferably at least 2, 3, 4, or more) of a conditional aggregation domain (CAD) a domain that interacts with itself in a ligand-reversible manner (i.e., in the presence of an inducing agent) using molecular biology methods known in the art and discussed elsewhere herein. The CAD domain may be the mutant domain isolated from the human FKBP12 (Phe³⁶ to Met) protein (as disclosed in V. M. Rivera., et al., Science, 287:826-830, (2000), or alternatively other proteins having domains with similar ligand-reversible, self-aggregation properties. As a principle of design the fusion protein vector would contain a furin cleavage sequence operably linked between the polynucleotides of the present invention and the CAD domains. Such a cleavage site would enable the proteolytic cleavage of the CAD domains from the polypeptide of the present invention subsequent to secretion from the ER and upon entry into the trans-Golgi (J. B. Denault, et al., FEBS Lett., 379:113, (1996)). Alternatively, the skilled artisan would recognize that any proteolytic cleavage sequence could be substituted for the furin sequence provided the substituted sequence is cleavable either endogenously (e.g., the furin sequence) or exogenously (e.g., post secretion, post purification, post production, etc.). The preferred sequence of each feature of the fusion protein construct, from the 5′ to 3′ direction with each feature being operably linked to the other, would be a promoter, signal sequence, “X” number of (CAD)x domains, the furin sequence (or other proteolytic sequence), and the coding sequence of the polypeptide of the present invention. The artisan would appreciate that the promotor and signal sequence, independent from the other, could be either the endogenous promotor or signal sequence of a polypeptide of the present invention, or alternatively, could be a heterologous signal sequence and promotor.

The specific methods described herein for controlling protein secretion levels through controlled ER aggregation are not meant to be limiting are would be generally applicable to any of the polynucleotides and polypeptides of the present invention, including variants, homologues, orthologs, and fragments therein.

### Example 17

Alteration of Protein Glycosylation Sites to Enhance Characteristics of Polypeptides of the Invention

Many eukaryotic cell surface and proteins are post-translationally processed to incorporate N-linked and O-linked carbohydrates (Kornfeld and Kornfeld (1985) Annu. Rev. Biochem. 54:631-64; Rademacher et al., (1988) Annu. Rev. Biochem. 57:785-838). Protein glycosylation is thought to serve a variety of functions including: augmentation of protein folding, inhibition of protein aggregation, regulation of intracellular trafficking to organelles, increasing resistance to proteolysis, modulation of protein antigenicity, and mediation of intercellular adhesion (Fieldler and Simons (1995) Cell, 81:309-312; Helenius (1994) Mol. Biol. Of the Cell 5:253-265; Olden et al., (1978) Cell, 13:461-473; Caton et al., (1982) Cell, 37:417-427; Alexamnder and Elder (1984), Science, 226:1328-1330; and Flack et al., (1994), J. Biol. Chem., 269:14015-14020). In higher organisms, the nature and extent of glycosylation can markedly affect the circulating half-life and bio-availability of proteins by mechanisms involving receptor mediated uptake and clearance (Ashwell and Morrell, (1974), Adv. Enzymol., 41:99-128; Ashwell and Harford (1982), Ann. Rev. Biochem., 51:531-54). Receptor systems have been identified that are thought to play a major role in the clearance of serum proteins through recognition of various carbohydrate structures on the glycoproteins (Stockert (1995), Physiol. Rev., 75:591-609; Kery et al., (1992), Arch. Biochem. Biophys., 298:49-55). Thus, production strategies resulting in incomplete attachment of terminal sialic acid residues might provide a means of shortening the bioavailability and half-life of glycoproteins. Conversely, expression strategies resulting in saturation of terminal sialic acid attachment sites might lengthen protein bioavailability and half-life.

In the development of recombinant glycoproteins for use as pharmaceutical products, for example, it has been speculated that the pharmacodynamics of recombinant proteins can be modulated by the addition or deletion of glycosylation sites from a glycoproteins primary structure (Berman and Lasky (1985a) Trends in Biotechnol., 3:51-53). However, studies have reported that the deletion of N-linked glycosylation sites often impairs intracellular transport and results in the intracellular accumulation of glycosylation site variants (Machamer and Rose (1988), J. Biol Chem., 263:5955-5960; Gallagher et al., (1992), J. Virology., 66:7136-7145; Collier et al., (1993), Biochem., 32:7818-7823; Claffey et al., (1995) Biochemica et Biophysica Acta, 1246:1-9; Dube et al., (1988), J. Biol. Chem. 263:17516-17521). While glycosylation site variants of proteins can be expressed intracellularly, it has proved difficult to recover useful quantities from growth conditioned cell culture medium.

Moreover, it is unclear to what extent a glycosylation site in one species will be recognized by another species glycosylation machinery. Due to the importance of glycosylation in protein metabolism, particularly the secretion and/or expression of the protein, whether a glycosylation signal is recognized may profoundly determine a proteins ability to be expressed, either endogenously or recombinately, in another organism (i.e., expressing a human protein in E.coli, yeast, or viral organisms; or an E. coli, yeast, or viral protein in human, etc.). Thus, it may be desirable to add, delete, or modify a glycosylation site, and possibly add a glycosylation site of one species to a protein of another species to improve the proteins functional, bioprocess purification, and/or structural characteristics (e.g., a polypeptide of the present invention).

A number of methods may be employed to identify the location of glycosylation sites within a protein. One preferred method is to run the translated protein sequence through the PROSITE computer program (Swiss Institute of Bioinformatics). Once identified, the sites could be systematically deleted, or impaired, at the level of the DNA using mutagenesis methodology known in the art and available to the skilled artisan, Preferably using PCR-directed mutagenesis (See Maniatis, Molecular Cloning: A Laboratory Manual, Cold Spring Harbor Press, Cold Spring, N.Y. (1982)). Similarly, glycosylation sites could be added, or modified at the level of the DNA using similar methods, preferably PCR methods (See, Maniatis, supra). The results of modifying the glycosylation sites for a particular protein (e.g., solubility, secretion potential, activity, aggregation, proteolytic resistance, etc.) could then be analyzed using methods know in the art.

The skilled artisan would acknowledge the existence of other computer algorithms capable of predicting the location of glycosylation sites within a protein. For example, the Motif computer program (Genetics Computer Group suite of programs) provides this function, as well.

### Example 18

Method of Enhancing the Biological Activity/Functional Characteristics of Invention Through Molecular Evolution

Although many of the most biologically active proteins known are highly effective for their specified function in an organism, they often possess characteristics that make them undesirable for transgenic, therapeutic, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered neurotransmitter receptor may be constitutively active upon binding of its cognate ligand. Alternatively, an engineered neurotransmitter receptor may be constitutively active in the absence of ligand binding. In yet another example, an engineered neurotransmitter receptor may be capable of being activated with less than all of the regulatory factors and/or conditions typically required for neurotransmitter receptor activation (e.g., ligand binding, phosphorylation, conformational changes, etc.). Such neurotransmitter receptors would be useful in screens to identify neurotransmitter receptor modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR reaction for all of the fragments—further diversifying the potential hybridization sites during the annealing step of the reaction.

A variety of reaction conditions could be utilized to carry-out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatmann) or could be purified using Microcon concentrators (Amicon) of the appropriate molecular weight cutoff, or could use oligonucleotide purification columns (Qiagen), in addition to other methods known in the art. If using DE81 ion-exchange paper, the 10-50 bp fragments could be eluted from said paper using 1M NaCl, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris(HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C. for 60 s; 94 C. for 30 s, 50-55 C. for 30 s, and 72 C. for 30 s using 30-45 cycles, followed by 72 C. for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C. for 30 s, 50 C. for 30 s, and 72 C. for 30 s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et al. (Nucl Acid Res., 25(6):1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homologue sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in US Patent No. 5,605,793; PCT Application No. WO 95/22625; PCT Application No. WO 97/20078; PCT Application No. WO 97/35966; and PCT Application No. WO 98/42832; PCT Application No. WO 00/09727 specifically provides methods for applying DNA shuffling to the identification of herbicide selective crops which could be applied to the polynucleotides and polypeptides of the present invention; additionally, PCT Application No. WO 00/12680 provides methods and compositions for generating, modifying, adapting, and optimizing polynucleotide sequences that confer detectable phenotypic properties on plant species; each of the above are hereby incorporated in their entirety herein for all purposes.

### Example 19

Method of Determining Alterations in a Gene Corresponding to a Polynucleotide

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO: 2. Suggested PCR conditions consist of 35 cycles at 95 degrees C. for 30 seconds; 60-120 seconds at 52-58 degrees C.; and 60-120 seconds at 70 degrees C., using buffer solutions described in Sidransky et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations is then cloned and sequenced to validate the results of the direct sequencing.

PCR products is cloned into T-tailed vectors as described in Holton et al., Nucleic Acids Research, 19:1156 (1991) and sequenced with T7 polymerase (United States Biochemical). Affected individuals are identified by mutations not present in unaffected individuals.

Genomic rearrangements are also observed as a method of determining alterations in a gene corresponding to a polynucleotide. Genomic clones isolated according to Example 2 are nick-translated with digoxigenindeoxy-uridine 5′-triphosphate (Boehringer Manheim), and FISH performed as described in Johnson et al., Methods Cell Biol. 35:73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 20

Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 ug/ml. The antibodies are either monoclonal or polyclonal and are produced by the method described elsewhere herein. The wells are blocked so that non-specific binding of the polypeptide to the well is reduced.

The coated wells are then incubated for>2 hours at RT with a sample containing the polypeptide. Preferably, serial dilutions of the sample should be used to validate results. The plates are then washed three times with deionized or distilled water to remove unbounded polypeptide.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 21

Formulation

The invention also provides methods of treatment and/or prevention diseases, disorders, and/or conditions (such as, for example, any one or more of the diseases or disorders disclosed herein) by administration to a subject of an effective amount of a Therapeutic. By therapeutic is meant a polynucleotides or polypeptides of the invention (including fragments and variants), agonists or antagonists thereof, and/or antibodies thereto, in combination with a pharmaceutically acceptable carrier type (e.g., a sterile carrier).

The Therapeutic will be formulated and dosed in a fashion consistent with good medical practice, taking into account the clinical condition of the individual patient (especially the side effects of treatment with the Therapeutic alone), the site of delivery, the method of administration, the scheduling of administration, and other factors known to practitioners. The “effective amount” for purposes herein is thus determined by such considerations.

As a general proposition, the total pharmaceutically effective amount of the Therapeutic administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the Therapeutic is typically administered at a dose rate of about 1 ug/kg/hour to about 50 ug/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Therapeutics can be administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intraarticular injection and infusion.

Therapeutics of the invention are also suitably administered by sustained-release systems. Suitable examples of sustained-release Therapeutics are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

Therapeutics of the invention may also be suitably administered by sustained-release systems. Suitable examples of sustained-release Therapeutics include suitable polymeric materials (such as, for example, semi-permeable polymer matrices in the form of shaped articles, e.g., films, or microcapsules), suitable hydrophobic materials (for example as an emulsion in an acceptable oil) or ion exchange resins, and sparingly soluble derivatives (such as, for example, a sparingly soluble salt).

Sustained-release matrices include polylactides (U.S. Pat. No. 3,773,919, EP 58,481), copolymers of L-glutamic acid and gamma-ethyl-L-glutamate (Sidman et al., Biopolymers 22:547-556 (1983)), poly (2-hydroxyethyl methacrylate) (Langer et al., J. Biomed. Mater. Res. 15:167-277 (1981), and Langer, Chem. Tech. 12:98-105 (1982)), ethylene vinyl acetate (Langer et al., Id.) or poly-D-(−)-3-hydroxybutyric acid (EP 133,988).

Sustained-release Therapeutics also include liposomally entrapped Therapeutics of the invention (see, generally, Langer, Science 249:1527-1533 (1990); Treat et al., in Liposomes in the Therapy of Infectious Disease and Cancer, Lopez-Berestein and Fidler (eds.), Liss, New York, pp. 317-327 and 353-365 (1989)). Liposomes containing the Therapeutic are prepared by methods known per se: DE 3,218,121; Epstein et al., Proc. Natl. Acad. Sci. (USA) 82:3688-3692 (1985); Hwang et al., Proc. Natl. Acad. Sci.(USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal Therapeutic.

In yet an additional embodiment, the Therapeutics of the invention are delivered by way of a pump (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)).

Other controlled release systems are discussed in the review by Langer (Science 249:1527-1533 (1990)).

For parenteral administration, in one embodiment, the Therapeutic is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to the Therapeutic.

Generally, the formulations are prepared by contacting the Therapeutic uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The Therapeutic will typically be formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any pharmaceutical used for therapeutic administration can be sterile. Sterility is readily accomplished by filtration through sterile filtration membranes (e.g., 0.2 micron membranes). Therapeutics generally are placed into a container having a sterile access port, for example, an intravenous solution bag or vial having a stopper pierceable by a hypodermic injection needle.

Therapeutics ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous Therapeutic solution, and the resulting mixture is lyophilized. The infusion solution is prepared by reconstituting the lyophilized Therapeutic using bacteriostatic Water-for-Injection.

The invention also provides a pharmaceutical pack or kit comprising one or more containers filled with one or more of the ingredients of the Therapeutics of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the Therapeutics may be employed in conjunction with other therapeutic compounds.

The Therapeutics of the invention may be administered alone or in combination with adjuvants. Adjuvants that may be administered with the Therapeutics of the invention include, but are not limited to, alum, alum plus deoxycholate (ImmunoAg), MTP-PE (Biocine Corp.), QS21 (Genentech, Inc.), BCG, and MPL. In a specific embodiment, Therapeutics of the invention are administered in combination with alum. In another specific embodiment, Therapeutics of the invention are administered in combination with QS-21. Further adjuvants that may be administered with the Therapeutics of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the Therapeutics of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

The Therapeutics of the invention may be administered alone or in combination with other therapeutic agents. Therapeutic agents that may be administered in combination with the Therapeutics of the invention, include but not limited to, other members of the TNF family, chemotherapeutic agents, antibiotics, steroidal and non-steroidal anti-inflammatories, conventional immunotherapeutic agents, cytokines and/or growth factors. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In one embodiment, the Therapeutics of the invention are administered in combination with members of the TNF family. TNF, TNF-related or TNF-like molecules that may be administered with the Therapeutics of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/33904), DR4 (International Publication No. WO 98/32856), TR5 (International Publication No. WO 98/30693), TR6 (International Publication No. WO 98/30694), TR7 (International Publication No. WO 98/41629), TRANK, TR9 (International Publication No. WO 98/56892), TR10 (International Publication No. WO 98/54202), 312C2 (International Publication No. WO 98/06842), and TR12, and soluble forms CD154, CD70, and CD153.

In certain embodiments, Therapeutics of the invention are administered in combination with antiretroviral agents, nucleoside reverse transcriptase inhibitors, non-nucleoside reverse transcriptase inhibitors, and/or protease inhibitors. Nucleoside reverse transcriptase inhibitors that may be administered in combination with the Therapeutics of the invention, include, but are not limited to, RETROVIR (zidovudine/AZT), VIDEX (didanosine/ddI), HIVID (zalcitabine/ddC), ZERIT (stavudine/d4T), EPIVIR (lamivudine/3TC), and COMBFVIR (zidovudine/lamivudine). Non-nucleoside reverse transcriptase inhibitors that may be administered in combination with the Therapeutics of the invention, include, but are not limited to, VIRAMUNE (nevirapine), RESCRIPTOR (delavirdine), and SUSTIVA (efavirenz). Protease inhibitors that may be administered in combination with the Therapeutics of the invention, include, but are not limited to, CRIXIVAN (indinavir), NORVIR (ritonavir), INVIRASE (saquinavir), and VIRACEPT (nelfinavir). In a specific embodiment, antiretroviral agents, nucleoside reverse transcriptase inhibitors, non-nucleoside reverse transcriptase inhibitors, and/or protease inhibitors may be used in any combination with Therapeutics of the invention to treat AIDS and/or to prevent or treat HIV infection.

In other embodiments, Therapeutics of the invention may be administered in combination with anti-opportunistic infection agents. Anti-opportunistic agents that may be administered in combination with the Therapeutics of the invention, include, but are not limited to, TRIMETHOPRIM-SULFAMETHOXAZOLE, DAPSONE, PENTAMIDINE, ATOVAQUONE, ISONIAZID, RIFAMPIN, PYRAZINAMIDE, ETHAMBUTOL, RIFABUTIN, CLARITHROMYCIN, AZITHROMYCIN, GANCICLOVIR, FOSCARNET, CIDOFOVIR, FLUCONAZOLE, ITRACONAZOLE, KETOCONAZOLE, ACYCLOVIR, FAMCICOLVIR, PYRIMETHAMINE, LEUCOVORIN, NEUPOGEN (filgrastim/G-CSF), and LEUKINE (sargramostim/GM-CSF). In a specific embodiment, Therapeutics of the invention are used in any combination with TRIMETHOPRIM-SULFAMETHOXAZOLE, DAPSONE, PENTAMIDINE, and/or ATOVAQUONE to prophylactically treat or prevent an opportunistic Pneumocystis carinii pneumonia infection. In another specific embodiment, Therapeutics of the invention are used in any combination with ISONIAZID, RIFAMPIN, PYRAZINAMIDE, and/or ETHAMBUTOL to prophylactically treat or prevent an opportunistic Mycobacterium avium complex infection. In another specific embodiment, Therapeutics of the invention are used in any combination with RIFABUTIN, CLARITHROMYCIN, and/or AZITHROMYCIN to prophylactically treat or prevent an opportunistic Mycobacterium tuberculosis infection. In another specific embodiment, Therapeutics of the invention are used in any combination with GANCICLOVIR, FOSCARNET, and/or CIDOFOVIR to prophylactically treat or prevent an opportunistic cytomegalovirus infection. In another specific embodiment, Therapeutics of the invention are used in any combination with FLUCONAZOLE, ITRACONAZOLE, and/or KETOCONAZOLE to prophylactically treat or prevent an opportunistic fungal infection. In another specific embodiment, Therapeutics of the invention are used in any combination with ACYCLOVIR and/or FAMCICOLVIR to prophylactically treat or prevent an opportunistic herpes simplex virus type I and/or type II infection. In another specific embodiment, Therapeutics of the invention are used in any combination with PYRIMETHAMINE and/or LEUCOVORIN to prophylactically treat or prevent an opportunistic Toxoplasma gondii infection. In another specific embodiment, Therapeutics of the invention are used in any combination with LEUCOVORIN and/or NEUPOGEN to prophylactically treat or prevent an opportunistic bacterial infection.

In a further embodiment, the Therapeutics of the invention are administered in combination with an antiviral agent. Antiviral agents that may be administered with the Therapeutics of the invention include, but are not limited to, acyclovir, ribavirin, amantadine, and remantidine.

In a further embodiment, the Therapeutics of the invention are administered in combination with an antibiotic agent. Antibiotic agents that may be administered with the Therapeutics of the invention include, but are not limited to, amoxicillin, beta-lactamases, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

Conventional nonspecific immunosuppressive agents, that may be administered in combination with the Therapeutics of the invention include, but are not limited to, steroids, cyclosporine, cyclosporine analogs, cyclophosphamide methylprednisone, prednisone, azathioprine, FK-506, 15-deoxyspergualin, and other immunosuppressive agents that act by suppressing the function of responding T cells.

In specific embodiments, Therapeutics of the invention are administered in combination with immunosuppressants. Immunosuppressants preparations that may be administered with the Therapeutics of the invention include, but are not limited to, ORTHOCLONE (OKT3), SANDIMMUNE/NEORAL/SANGDYA (cyclosporin), PROGRAF (tacrolimus), CELLCEPT (mycophenolate), Azathioprine, glucorticosteroids, and RAPAMUNE (sirolimus). In a specific embodiment, immunosuppressants may be used to prevent rejection of organ or bone marrow transplantation.

In an additional embodiment, Therapeutics of the invention are administered alone or in combination with one or more intravenous immune globulin preparations. Intravenous immune globulin preparations that may be administered with the Therapeutics of the invention include, but not limited to, GAMMAR, IVEEGAM, SANDOGLOBULIN, GAMMAGARD S/D, and GAMIMUNE. In a specific embodiment, Therapeutics of the invention are administered in combination with intravenous immune globulin preparations in transplantation therapy (e.g., bone marrow transplant).

In an additional embodiment, the Therapeutics of the invention are administered alone or in combination with an anti-inflammatory agent. Anti-inflammatory agents that may be administered with the Therapeutics of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzylamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the Therapeutics of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In a specific embodiment, Therapeutics of the invention are administered in combination with CHOP (cyclophosphamide, doxorubicin, vincristine, and prednisone) or any combination of the components of CHOP. In another embodiment, Therapeutics of the invention are administered in combination with Rituximab. In a further embodiment, Therapeutics of the invention are administered with Rituxmab and CHOP, or Rituxmab and any combination of the components of CHOP.

In an additional embodiment, the Therapeutics of the invention are administered in combination with cytokines. Cytokines that may be administered with the Therapeutics of the invention include, but are not limited to, IL2, IL3, IL4, IL5, IL6, IL7, IL10, IL12, IL13, IL15, anti-CD40, CD40L, IFN-gamma and TNF-alpha. In another embodiment, Therapeutics of the invention may be administered with any interleukin, including, but not limited to, IL-1alpha, IL-1beta, IL-2, IL-3, IL-4, IL-5, IL-6, IL-7, IL-8, IL-9, IL-10, IL-11, IL-12, EL-13, IL-14, IL-15, IL-16, IL-17, IL-18, IL-19, IL-20, and IL-21.

In an additional embodiment, the Therapeutics of the invention are administered in combination with angiogenic proteins. Angiogenic proteins that may be administered with the Therapeutics of the invention include, but are not limited to, Glioma Derived Growth Factor (GDGF), as disclosed in European Patent Number EP-399816; Platelet Derived Growth Factor-A (PDGF-A), as disclosed in European Patent Number EP-6821 10; Platelet Derived Growth Factor-B (PDGF-B), as disclosed in European Patent Number EP-282317; Placental Growth Factor (PDGF), as disclosed in International Publication Number WO 92/06194; Placental Growth Factor-2 (PDGF-2), as disclosed in Hauser et al., Growth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B (VEGF-3); Vascular Endothelial Growth Factor B-186 (VEGF-B186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the Therapeutics of the invention are administered in combination with hematopoietic growth factors. Hematopoietic growth factors that may be administered with the Therapeutics of the invention include, but are not limited to, LEUKINE (SARGRAMOSTIM) and NEUPOGEN (FILGRASTIM).

In an additional embodiment, the Therapeutics of the invention are administered in combination with Fibroblast Growth Factors. Fibroblast Growth Factors that may be administered with the Therapeutics of the invention include, but are not limited to, FGF-1, FGF-2, FGF-3, FGF-4, FGF-5, FGF-6, FGF-7, FGF-8, FGF-9, FGF-10, FGF-11, FGF-12, FGF-13, FGF-14, and FGF-15.

In a specific embodiment, formulations of the present invention may further comprise antagonists of P-glycoprotein (also referred to as the multiresistance protein, or PGP), including antagonists of its encoding polynucleotides (e.g., antisense oligonucleotides, ribozymes, zinc-finger proteins, etc.). P-glycoprotein is well known for decreasing the efficacy of various drug administrations due to its ability to export intracellular levels of absorbed drug to the cell exterior. While this activity has been particularly pronounced in cancer cells in response to the administration of chemotherapy regimens, a variety of other cell types and the administration of other drug classes have been noted (e.g., T-cells and anti-HIV drugs). In fact, certain mutations in the PGP gene significantly reduces PGP function, making it less able to force drugs out of cells. People who have two versions of the mutated gene—one inherited from each parent—have more than four times less PGP than those with two normal versions of the gene. People may also have one normal gene and one mutated one. Certain ethnic populations have increased incidence of such PGP mutations. Among individuals from Ghana, Kenya, the Sudan, as well as African Americans, frequency of the normal gene ranged from 73% to 84%. In contrast, the frequency was 34% to 59% among British whites, Portuguese, Southwest Asian, Chinese, Filipino and Saudi populations. As a result, certain ethnic populations may require increased administration of PGP antagonist in the formulation of the present invention to arrive at the an efficacious dose of the therapeutic (e.g., those from African descent). Conversely, certain ethnic populations, particularly those having increased frequency of the mutated PGP (e.g., of Caucasian descent, or non-African descent) may require less pharmaceutical compositions in the formulation due to an effective increase in efficacy of such compositions as a result of the increased effective absorption (e.g., less PGP activity) of said composition.

Moreover, in another specific embodiment, formulations of the present invention may further comprise antagonists of OATP2 (also referred to as the multiresistance protein, or MRP2), including antagonists of its encoding polynucleotides (e.g., antisense oligonucleotides, ribozymes, zinc-finger proteins, etc.). The invention also further comprises any additional antagonists known to inhibit proteins thought to be attributable to a multidrug resistant phenotype in proliferating cells.

Preferred antagonists that formulations of the present may comprise include the potent P-glycoprotein inhibitor elacridar, and/or LY-335979. Other P-glycoprotein inhibitors known in the art are also encompassed by the present invention.

In additional embodiments, the Therapeutics of the invention are administered in combination with other therapeutic or prophylactic regimens, such as, for example, radiation therapy.

### Example 22

Method of Treating Decreased Levels of the Polypeptide

The present invention relates to a method for treating an individual in need of an increased level of a polypeptide of the invention in the body comprising administering to such an individual a composition comprising a therapeutically effective amount of an agonist of the invention (including polypeptides of the invention). Moreover, it will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a Therapeutic comprising an amount of the polypeptide to increase the activity level of the polypeptide in such an individual.

For example, a patient with decreased levels of a polypeptide receives a daily dose 0.1-100 ug/kg of the polypeptide for six consecutive days. Preferably, the polypeptide is in the secreted form. The exact details of the dosing scheme, based on administration and formulation, are provided herein.

### Example 23

Method of Treating Increased Levels of the Polypeptide

The present invention also relates to a method of treating an individual in need of a decreased level of a polypeptide of the invention in the body comprising administering to such an individual a composition comprising a therapeutically effective amount of an antagonist of the invention (including polypeptides and antibodies of the invention).

In one example, antisense technology is used to inhibit production of a polypeptide of the present invention. This technology is one example of a method of decreasing levels of a polypeptide, preferably a secreted form, due to a variety of etiologies, such as cancer. For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided herein.

### Example 24

Method of Treatment Using Gene Therapy—Ex Vivo

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37 degree C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth herein using primers and having appropriate restriction sites and initiation/stop codons, if necessary. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector has the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 25

Gene Therapy Using Endogenous Genes Corresponding to Polynucleotides of the Invention

Another method of gene therapy according to the present invention involves operably associating the endogenous polynucleotide sequence of the invention with a promoter via homologous recombination as described, for example, in U.S. Pat. No. 5,641,670, issued Jun. 24, 1997; International Publication NO: WO 96/29411, published Sep. 26, 1996; International Publication NO: WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous polynucleotide sequence, flanking the promoter. The targeting sequence will be sufficiently near the 5′ end of the polynucleotide sequence so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous polynucleotide sequence. This results in the expression of polynucleotide corresponding to the polynucleotide in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×106 cells/ml. Electroporation should be performed immediately following resuspension.

Plasmid DNA is prepared according to standard techniques. For example, to construct a plasmid for targeting to the locus corresponding to the polynucleotide of the invention, plasmid pUC18 (MBI Fermentas, Amherst, N.Y.) is digested with HindIII. The CMV promoter is amplified by PCR with an XbaI site on the 5′ end and a BamHI site on the 3′end. Two non-coding sequences are amplified via PCR: one non-coding sequence (fragment 1) is amplified with a HindIII site at the 5′ end and an Xba site at the 3′end; the other non-coding sequence (fragment 2) is amplified with a BamHI site at the 5′end and a HindIII site at the 3′end. The CMV promoter and the fragments (1 and 2) are digested with the appropriate enzymes (CMV promoter—XbaI and BamHI; fragment 1—XbaI; fragment 2—BamHI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5×106 cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37 degree C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 26

Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata et al., Cardiovasc. Res. 35(3):470-479 (1997); Chao et al., Pharmacol. Res. 35(6):517-522 (1997); Wolff, Neuromuscul. Disord. 7(5):314-318 (1997); Schwartz et al., Gene Ther. 3(5):405-411 (1996); Tsurumi et al., Circulation 94(12):3281-3290 (1996) (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 27

Transgenic Animals

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR(RT-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 28

Knock-Out Animals

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 29

Production of an Antibody

a) Hybridoma Technology

The antibodies of the present invention can be prepared by a variety of methods. (See, Current Protocols, Chapter 2.) As one example of such methods, cells expressing HNTTBMY1 are administered to an animal to induce the production of sera containing polyclonal antibodies. In a preferred method, a preparation of HNTTBMY1 protein is prepared and purified to render it substantially free of natural contaminants. Such a preparation is then introduced into an animal in order to produce polyclonal antisera of greater specific activity.

Monoclonal antibodies specific for protein HNTTBMY1 are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N. Y., pp. 563-681 (1981)). In general, an animal (preferably a mouse) is immunized with HNTTBMY1 polypeptide or, more preferably, with a secreted HNTTBMY1 polypeptide-expressing cell. Such polypeptide-expressing cells are cultured in any suitable tissue culture medium, preferably in Earle's modified Eagle's medium supplemented with 10% fetal bovine serum (inactivated at about 56° C.), and supplemented with about 10 μg/ml of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the HNTTBMY1 polypeptide.

Alternatively, additional antibodies capable of binding to HNTTBMY1 polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the HNTTBMY1 protein-specific antibody can be blocked by HNTTBMY1. Such antibodies comprise anti-idiotypic antibodies to the HNTTBMY1 protein-specific antibody and are used to immunize an animal to induce formation of further HNTTBMY1 protein-specific antibodies.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

b) Isolation of Antibody Fragments Directed Against HNTTBMY1 from a Library of scFvs

Naturally occurring V-genes isolated from human PBLs are constructed into a library of antibody fragments which contain reactivities against HNTTBMY1 to which the donor may or may not have been exposed (see e.g., U.S. Pat. No. 5,885,793 incorporated herein by reference in its entirety).

Rescue of the Library. A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to inoculate 50 ml of 2×TY-AMP-GLU, 2×108 TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 μg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/ml (2×TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 μg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

### Example 30

Identification and Cloning of VH and VL Domains of Antibodies Directed Against the HNTTBMY1 Polypeptide

VH and VL domains may be identified and cloned from cell lines expressing an antibody directed against a HNTTBMY1 epitope by performing PCR with VH and VL specific primers on cDNA made from the antibody expressing cell lines. Briefly, RNA is isolated from the cell lines and used as a template for RT-PCR designed to amplify the VH and VL domains of the antibodies expressed by the EBV cell lines. Cells may be lysed using the TRIzol reagent (Life Technologies, Rockville, Md.) and extracted with one fifth volume of chloroform. After addition of chloroform, the solution is allowed to incubate at room temperature for 10 minutes, and then centrifuged at 14,000 rpm for 15 minutes at 4 C. in a tabletop centrifuge. The supernatant is collected and RNA is precipitated using an equal volume of isopropanol. Precipitated RNA is pelleted by centrifuging at 14,000 rpm for 15 minutes at 4 C. in a tabletop centrifuge.

Following centrifugation, the supernatant is discarded and washed with 75% ethanol. Following the wash step, the RNA is centrifuged again at 800 rpm for 5 minutes at 4 C. The supernatant is discarded and the pellet allowed to air dry. RNA is the dissolved in DEPC water and heated to 60 C. for 10 minutes. Quantities of RNA can be determined using optical density measurements. cDNA may be synthesized, according to methods well-known in the art and/or described herein, from 1.5-2.5 micrograms of RNA using reverse transcriptase and random hexamer primers. cDNA is then used as a template for PCR amplification of VH and VL domains.

Primers used to amplify VH and VL genes are shown below. Typically a PCR reaction makes use of a single 5′primer and a single 3′primer. Sometimes, when the amount of available RNA template is limiting, or for greater efficiency, groups of 5′ and/or 3′primers may be used. For example, sometimes all five VH-5′primers and all JH3′primers are used in a single PCR reaction. The PCR reaction is carried out in a 50 microliter volume containing 1× PCR buffer, 2 mM of each dNTP, 0.7 units of High Fidelity Taq polymerase, 5′primer mix, 3′primer mix and 7.5 microliters of cDNA. The 5′ and 3′primer mix of both VH and VL can be made by pooling together 22 pmole and 28 pmole, respectively, of each of the individual primers. PCR conditions are: 96 C. for 5 minutes; followed by 25 cycles of 94 C. for 1 minute, 50 C. for 1 minute, and 72 C. for 1 minute; followed by an extension cycle of 72 C. for 10 minutes. After the reaction has been completed, sample tubes may be stored at 4 C.

Primer Sequences Used to Amplify VH domains

| Primer name | Primer Sequence | SEQ ID NO: |
|---|---|---|
| Hu VH1-5′ | CAGGTGCAGCTGGTGCAGTCTGG | 60 |
| Hu VH2-5′ | CAGGTCAACTTAAGGGAGTCTGG | 61 |
| Hu VH3-5′ | GAGGTGCAGCTGGTGGAGTCTGG | 62 |
| Hu VH4-5′ | CAGGTGCAGCTGCAGGAGTCGGG | 63 |
| Hu VH5-5′ | GAGGTGCAGCTGTTGCAGTCTGC | 64 |
| Hu VH6-5′ | CAGGTACAGCTGCAGCAGTCAGG | 65 |
| Hu JH1-5′ | TGAGGAGACGGTGACCAGGGTGCC | 66 |
| Hu JH3-5′ | TGAAGAGACGGTGACCATTGTCCC | 67 |
| Hu JH4-5′ | TGAGGAGACGGTGACCAGGGTTCC | 68 |
| Hu JH6-5′ | TGAGGAGACGGTGACCGTGGTCCC | 69 |

Primer Sequences Used to Amplify VL domains

| Primer name | Primer Sequence | SEQ ID NO: |
|---|---|---|
| Hu Vkappa1-5′ | GACATCCAGATGACCCAGTCTCC | 70 |
| Hu Vkappa2a-5′ | GATGTTGTGATGACTCAGTCTCC | 71 |
| Hu Vkappa2b-5′ | GATATTGTGATGACTCAGTCTCC | 72 |
| Hu Vkappa3-5′ | GAAATTGTGTTGACGCAGTCTCC | 73 |
| Hu Vkappa4-5′ | GACATCGTGATGACCCAGTCTCC | 74 |
| Hu Vkappa5-5′ | GAAACGACACTCACGCAGTCTCC | 75 |
| Hu Vkappa6-5′ | GAAATTGTGCTGACTCAGTCTCC | 76 |
| Hu Vlambda1-5′ | CAGTCTGTGTTGACGCAGCCGCC | 77 |
| Hu Vlambda2-5′ | CAGTCTGCCCTGACTCAGCCTGC | 78 |
| Hu Vlambda3-5′ | TCCTATGTGCTGACTCAGCCACC | 79 |
| Hu Vlambda3b-5′ | TCTTCTGAGCTGACTCAGGACCC | 80 |
| Hu Vlambda4-5′ | CACGTTATACTGACTCAACCGCC | 81 |
| Hu Vlambda5-5′ | CAGGCTGTGCTCACTCAGCCGTC | 82 |
| Hu Vlambda6-5′ | AATTTTATGCTGACTCAGCCCCA | 83 |
| Hu Jkappa1-3′ | ACGTTTGATTTCCACCTTGGTCCC | 84 |
| Hu Jkappa2-3′ | ACGTTTGATCTCCAGCTTGGTCCC | 85 |
| Hu Jkappa3-3′ | ACGTTTGATATCCACTTTGGTCCC | 86 |
| Hu Jkappa4-3′ | ACGTTTGATCTCCACCTTGGTCCC | 87 |
| Hu Jkappa5-3′ | ACGTTTAATCTCCAGTCGTGTCCC | 88 |
| Hu Vlambda1-3′ | CAGTCTGTGTTGACGCAGCCGCC | 89 |
| Hu Vlambda2-3′ | CAGTCTGCCCTGACTCAGCCTGC | 90 |
| Hu Vlambda3-3′ | TCCTATGTGCTGACTCAGCCACC | 91 |
| Hu Vlambda3b-3′ | TCTTCTGAGCTGACTCAGGACCC | 92 |
| Hu Vlambda4-3′ | CACGTTATACTGACTCAACCGCC | 93 |
| Hu Vlambda5-3′ | CAGGCTGTGCTCACTCAGCCGTC | 94 |
| Hu Vlambda6-3′ | AATTTTATGCTGACTCAGCCCCA | 95 |

PCR samples are then electrophoresed on a 1.3% agarose gel. DNA bands of the expected sizes (−506 base pairs for VH domains, and 344 base pairs for VL domains) can be cut out of the gel and purified using methods well known in the art and/or described herein.

Purified PCR products can be ligated into a PCR cloning vector (TA vector from Invitrogen Inc., Carlsbad, Calif.). Individual cloned PCR products can be isolated after transfection of E. coli and blue/white color selection. Cloned PCR products may then be sequenced using methods commonly known in the art and/or described herein.

The PCR bands containing the VH domain and the VL domains can also be used to create full-length Ig expression vectors. VH and VL domains can be cloned into vectors containing the nucleotide sequences of a heavy (e.g., human IgG1 or human IgG4) or light chain (human kappa or human ambda) constant regions such that a complete heavy or light chain molecule could be expressed from these vectors when transfected into an appropriate host cell. Further, when cloned heavy and light chains are both expressed in one cell line (from either one or two vectors), they can assemble into a complete functional antibody molecule that is secreted into the cell culture medium. Methods using polynucleotides encoding VH and VL antibody domain to generate expression vectors that encode complete antibody molecules are well known within the art.

### Example 30

Biological Effects of HNTTBMY1 Polypeptides of the Invention

Astrocyte and Neuronal Assays

Recombinant polypeptides of the invention, expressed in Escherichia coli and purified as described above, can be tested for activity in promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate a polypeptide of the invention's activity on these cells.

Moreover, previous reports describing the biological effects of FGF-2 (basic FGF) on cortical or hippocampal neurons in vitro have demonstrated increases in both neuron survival and neurite outgrowth (Walicke et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of a polypeptide of the invention to induce neurite outgrowth can be compared to the response achieved with FGF-2 using, for example, a thymidine incorporation assay.

Fibroblast and Endothelial Cell Assays

Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated for one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE2 assays, the human lung fibroblasts are cultured at 5,000 cells/well in a 96-well plate for one day. After a medium change to 0.1% BSA basal medium, the cells are incubated with FGF-2 or polypeptides of the invention with or without IL-1( for 24 hours. The supernatants are collected and assayed for PGE2 by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or with or without polypeptides of the invention IL-1( for 24 hours. The supernatants are collected and assayed for IL-6 by ELISA kit (Endogen, Cambridge, Mass.).

Human lung fibroblasts are cultured with FGF-2 or polypeptides of the invention for 3 days in basal medium before the addition of Alamar Blue to assess effects on growth of the fibroblasts. FGF-2 should show a stimulation at 10-2500 ng/ml which can be used to compare stimulation with polypeptides of the invention.

Parkinson Models

The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP+) and released. Subsequently, MPP+ is actively accumulated in dopaminergic neurons by the high-affinity reuptake transporter for dopamine. MPP+ is then concentrated in mitochondria by the electrochemical gradient and selectively inhibits nicotidamide adenine diphosphate: ubiquinone oxidoreductionase (complex I), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, polypeptides of the invention can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of a polypeptide of the invention is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm2 on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopaminergic neurons, immunohistochemical staining. Dissociated cell cultures are prepared from embryonic rats. The culture medium is changed every third day and the factors are also added at that time.

Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if a polypeptide of the invention acts to prolong the survival of dopaminergic neurons, it would suggest that the polypeptide may be involved in Parkinson's Disease.

One skilled in the art could easily modify the exemplified studies to test the activity of polynucleotides of the invention (e.g., gene therapy), agonists, and/or antagonists of polynucleotides or polypeptides of the invention.

### Example 31

Method of Creating N- and C-Terminal Deletion Mutants Corresponding to the HNTTBMY1 Polypeptide of the Present Invention

The present invention encompasses the creation of N- and C-terminal deletion mutants, in addition to any combination of N- and C-terminal deletions thereof, corresponding to the HNTTBMY1 polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Such N-terminus or C-terminus deletions of a polypeptide of the present invention may, in fact, result in a significant increase in one or more of the biological activities of the polypeptide(s). For example, biological activity of many polypeptides are governed by the presence of regulatory domains at either one or both termini. Such regulatory domains effectively inhibit the biological activity of such polypeptides in lieu of an activation event (e.g., binding to a cognate ligand or receptor, phosphorylation, proteolytic processing, etc.). Thus, by eliminating the regulatory domain of a polypeptide, the polypeptide may effectively be rendered biologically active in the absence of an activation event.

Briefly, using the isolated cDNA clone encoding the full-length HNTTBMY1 polypeptide sequence (as described in herein), appropriate primers of about 15-25 nucleotides derived from the desired 5′ and 3′ positions of SEQ ID NO: 2 may be designed to PCR amplify, and subsequently clone, the intended N- and/or C-terminal deletion mutant. Such primers could comprise, for example, an initiation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozac sequences, or other sequences discussed and/or referenced herein.

For example, in the case of the to L727 N-terminal deletion mutant, the following primers could be used to amplify a cDNA fragment corresponding to this deletion mutant:

| | | |
|---|---|---|
| 5′Primer | 5′-GCAGCA GCGGCCGC TACATCCTGGCCCAGATTGGCTTC-3′ | (SEQ ID NO:31) |
| | NotI | |
| 3′Primer | 5′-GCAGCA GTCGAC CAGCTCCGACTCAGGGGTGCTGGCC-3′ | (SEQ ID NO:32) |
| | SalI | |

For example, in the case of the M1 to R641 C-terminal deletion mutant, the following primers could be used to amplify a cDNA fragment corresponding to this deletion mutant:

| | | |
|---|---|---|
| 5′Primer | 5′-GCAGCA GCGGCCGC ATGCCGAAGAACAGCAAAGTGACCC-3′ | (SEQ ID NO:33) |
| | NotI | |
| 3′Primer | 5′-GCAGCA GTCGAC GAAGTGCCGCAGGACGAACACCAC-3′ | SEQ ID NO:34) |
| | SalI | |

Representative PCR amplification conditions are provided below, although the skilled artisan would appreciate that other conditions may be required for efficient amplification. A 100 ul PCR reaction mixture may be prepared using 10 ng of the template DNA (cDNA clone of HNTTBMY1), 200 uM 4dNTPs, 1 uM primers, 0.25U Taq DNA polymerase (PE), and standard Taq DNA polymerase buffer. Typical PCR cycling condition are as follows:

20-25 cycles:45 sec, 93 degrees

2 min, 50 degrees

2 min, 72 degrees

1 cycle: 10 min, 72 degrees

After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E. coli cells using methods provided herein and/or otherwise known in the art.

The 5′ primer sequence for amplifying any additional N-terminal deletion mutants may be determined by reference to the following formula:

(S+(X*3)) to ((S+(X*3))+25),

wherein ‘S’ is equal to the nucleotide position of the initiating start codon of the HNTTBMY1 gene (SEQ ID NO: 2), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO: 2. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozac sequences, etc.).

The 3′ primer sequence for amplifying any additional N-terminal deletion mutants may be determined by reference to the following formula:

(S+(X*3)) to ((S+(X*3))−25),

wherein ‘S’ is equal to the nucleotide position of the initiating start codon of the HNTTBMY1 gene (SEQ ID NO: 2), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO: 2. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

In preferred embodiments, the following N-terminal HNTTBMY1 deletion polypeptides are encompassed by the present invention: M1-L727, P2-L727, K3-L727, N4-L727, S5-L727, K6-L727, V7-L727, T8-L727, Q9-L727, R10-L727, E11-L727, H12-L727, S13-L727, S14-L727, E15-L727, H16-L727, V17-L727, T18-L727, E19-L727, S20-L727, V21-L727, A22-L727, D23-L727, L24-L727, L25-L727, A26-L727, L27-L727, E28-L727, E29-L727, P30-L727, V31-L727, D32-L727, Y33-L727, K34-L727, Q35-L727, S36-L727, V37-L727, L38-L727, N39-L727, V40-L727, A41-L727, G42-L727, E43-L727, A44-L727, G45-L727, G46-L727, K47-L727, Q48-L727, K49-L727, A50-L727, V5 1-L727, E52-L727, E53-L727, E54-L727, L55-L727, D56-L727, T57-L727, E58-L727, D59-L727, R60-L727, P61-L727, A62-L727, W63-L727, N64-L727, S65-L727, K66-L727, L67-L727, Q68-L727, Y69-L727, I70-L727, L71-L727, A72-L727, Q73-L727, 174-L727, G75-L727, F76-L727, S77-L727, V78-L727, G79-L727, L80-L727, G81-L727, N82-L727, 183-L727, W84-L727, R85-L727, F86-L727, P87-L727, Y88-L727, L89-L727, C90-L727, Q91-L727, K92-L727, N93-L727, G94-L727, G95-L727, G96-L727, A97-L727, Y98-L727, L99-L727, V100-L727, P101-L727, Y102-L727, L103-L727, V104-L727, L105-L727, L106-L727, I107-L727, I108-L727, I109-L727, G110-L727, I111-L727, P112-L727, L113-L727, F114-L727, F115-L727, L116-L727, E117-L727, L118-L727, A119-L727, V120-L727, G121-L727, Q122-L727, R123-L727, 1124-L727, R125-L727, R126-L727, G127-L727, S128-L727, I129-L727, G130-L727, V131-L727, W132-L727, H133-L727, Y134-L727, I135-L727, C136-L727, P137-L727, R138-L727, L139-L727, G140-L727, G141-L727, I142-L727, G143-L727, F144-L727, S145-L727, S146-L727, C147-L727, I148-L727, V149-L727, C150-L727, L151-L727, F152-L727, V153-L727, G154-L727, L155-L727, Y156-L727, Y157-L727, N158-L727, V159-L727, I160-L727, I161-L727, G162-L727, W163-L727, S 164-L727, I165-L727, F166-L727, Y167-L727, F168-L727, F169-L727, K170-L727, S171-L727, F172-L727, Q173-L727, Y174-L727, P175-L727, L176-L727, P177-L727, W178-L727, S179-L727, E180-L727, C181-L727, P182-L727, V183-L727, V184-L727, R185-L727, N186-L727, G187-L727, S188-L727, V189-L727, A190-L727, V191-L727, V192-L727, E193-L727, A194-L727, E195-L727, C196-L727, E197-L727, K198-L727, S199-L727, S200-L727, A201-L727, T202-L727, T203-L727, Y204-L727, F205-L727, W206-L727, Y207-L727, R208-L727, E209-L727, A210-L727, L211-L727, D212-L727, I213-L727, S214-L727, D215-L727, S216-L727, I217-L727, S218-L727, E219-L727, S220-L727, G221-L727, G222-L727, L223-L727, N224-L727, W225-L727, K226-L727, M227-L727, T228-L727, L229-L727, C230-L727, L231-L727, L232-L727, V233-L727, A234-L727, W235-L727, S236-L727, I237-L727, V238-L727, G239-L727, M240-L727, A241-L727, V242-L727, V243-L727, K244-L727, G245-L727, 1246-L727, Q247-L727, S248-L727, S249-L727, G250-L727, K251-L727, V252-L727, M253-L727, Y254-L727, F255-L727, S256-L727, S257-L727, L258-L727, F259-L727, P260-L727, Y261-L727, V262-L727, V263-L727, L264-L727, A265-L727, C266-L727, F267-L727, L268-L727, V269-L727, R270-L727, G271-L727, L272-L727, L273-L727, L274-L727, R275-L727, G276-L727, A277-L727, V278-L727, D279-L727, G280-L727, 1281-L727, L282-L727, H283-L727, M284-L727, F285-L727, T286-L727, P287-L727, K288-L727, L289-L727, D290-L727, K291-L727, M292-L727, L293-L727, D294-L727, P295-L727, Q296-L727, V297-L727, W298-L727, R299-L727, E300-L727, A301-L727, A302-L727, T303-L727, Q304-L727, V305-L727, F306-L727, F307-L727, A308-L727, L309-L727, G310-L727, L311-L727, G312-L727, F313-L727, G314-L727, G315-L727, V316-L727, 1317-L727, A318-L727, F319-L727, S320-L727, S321-L727, Y322-L727, N323-L727, K324-L727, Q325-L727, D326-L727, N327-L727, N328-L727, C329-L727, H330-L727, F331-L727, D332-L727, A333-L727, A334-L727, L335-L727, V336-L727, S337-L727, F338-L727, 1339-L727, N340-L727, F341-L727, F342-L727, T343-L727, S344-L727, V345-L727, L346-L727, A347-L727, T348-L727, L349-L727, V350-L727, V351-L727, F352-L727, A353-L727, V354-L727, L355-L727, G356-L727, F357-L727, K358-L727, A359-L727, N360-L727, I361-L727, M362-L727, N363-L727, E364-L727, K365-L727, C366-L727, V367-L727, V368-L727, E369-L727, N370-L727, A371-L727, E372-L727, K373-L727, I374-L727, L375-L727, G376-L727, Y377-L727, L378-L727, N379-L727, T380-L727, N381-L727, V382-L727, L383-L727, S384-L727, R385-L727, D386-L727, L387-L727, I388-L727, P389-L727, P390-L727, H391-L727, V392-L727, N393-L727, F394-L727, S395-L727, H396-L727, L397-L727, T398-L727, T399-L727, K400-L727, D401-L727, Y402-L727, M403-L727, E404-L727, M405-L727, Y406-L727, N407-L727, V408-L727, 1409-L727, M410-L727, T411-L727, V412-L727, K413-L727, E414-L727, D415-L727, Q416-L727, F417-L727, S418-L727, A419-L727, L420-L727, G421-L727, L422-L727, D423-L727, P424-L727, C425-L727, L426-L727, I427-L727, E428-L727, D429-L727, E430-L727, L431-L727, D432-L727, K433-L727, S434-L727, V435-L727, Q436-L727, G437-L727, T438-L727, G439-L727, L440-L727, A441-L727, F442-L727, 1443-L727, A444-L727, F445-L727, T446-L727, E447-L727, A448-L727, M449-L727, T450-L727, H451-L727, F452-L727, P453-L727, A454-L727, S455-L727, P456-L727, F457-L727, W458-L727, S459-L727, V460-L727, M461-L727, F462-L727, F463-L727, L464-L727, M465-L727, L466-L727, I467-L727, N468-L727, L469-L727, G470-L727, L471-L727, G472-L727, S473-L727, M474-L727, 1475-L727, G476-L727, T477-L727, M478-L727, A479-L727, G480-L727, 1481-L727, T482-L727, T483-L727, P484-L727, I485-L727, I486-L727, D487-L727, T488-L727, F489-L727, K490-L727, V491-L727, P492-L727, K493-L727, E494-L727, M495-L727, F496-L727, T497-L727, V498-L727, G499-L727, C500-L727, C501-L727, V502-L727, F503-L727, A504-L727, F505-L727, L506-L727, V507-L727, G508-L727, L509-L727, L510-L727, F511-L727, V512-L727, Q513-L727, R514-L727, S515-L727, G516-L727, N517-L727, Y518-L727, F519-L727, V520-L727, T521-L727, M522-L727, F523-L727, D524-L727, D525-L727, Y526-L727, S527-L727, A528-L727, T529-L727, L530-L727, P531-L727, L532-L727, T533-L727, L534-L727, 1535-L727, V536-L727, 1537-L727, L538-L727, E539-L727, N540-L727, 1541-L727, A542-L727, V543-L727, A544-L727, W545-L727, 1546-L727, Y547-L727, G548-L727, T549-L727, K550-L727, K551-L727, F552-L727, M553-L727, Q554-L727, E555-L727, L556-L727, T557-L727, E558-L727, M559-L727, L560-L727, G561-L727, F562-L727, R563-L727, P564-L727, Y565-L727, R566-L727, F567-L727, Y568-L727, F569-L727, Y570-L727, M571-L727, W572-L727, K573-L727, F574-L727, V575-L727, S576-L727, P577-L727, L578-L727, C579-L727, M580-L727, A581-L727, V582-L727, L583-L727, T584-L727, T585-L727, A586-L727, S587-L727, I588-L727, I589-L727, Q590-L727, L591-L727, G592-L727, V593-L727, T594-L727, P595-L727, P596-L727, G597-L727, Y598-L727, S599-L727, A600-L727, W601-L727, I602-L727, K603-L727, E604-L727, E605-L727, A606-L727, A607-L727, E608-L727, R609-L727, Y610-L727, L611-L727, Y612-L727, F613-L727, P614-L727, N615-L727, W616-L727, A617-L727, M618-L727, A619-L727, L727, L621-L727, 1622-L727, T623-L727, L624-L727, 1625-L727, V626-L727, V627-L727, A628-L727, T629-L727, L630-L727, P631-L727, 1632-L727, P633-L727, V634-L727, V635-L727, F636-L727, V637-L727, L638-L727, R639-L727, H640-L727, F641-L727, H642-L727, L643-L727, L644-L727, S645-L727, D646-L727, G647-L727, S648-L727, N649-L727, T650-L727, L651-L727, S652-L727, V653-L727, S654-L727, Y655-L727, K656-L727, K657-L727, G658-L727, R659-L727, M660-L727, M661-L727, K662-L727, D663-L727, 1664-L727, S665-L727, N666-L727, L667-L727, E668-L727, E669-L727, N670-L727, D671-L727, E672-L727, T673-L727, R674-L727, F675-L727, 1676-L727, L677-L727, S678-L727, K679-L727, V680-L727, P681-L727, S682-L727, E683-L727, A684-L727, P685-L727, S686-L727, P687-L727, M688-L727, P689-L727, T690-L727, H691-L727, R692-L727, S693-L727, Y694-L727, L695-L727, G696-L727, P697-L727, G698-L727, S699-L727, T700-L727, S701-L727, P702-L727, L703-L727, E704-L727, T705-L727, S706-L727, G707-L727, N708-L727, P709-L727, N710-L727, G711-L727, R712-L727, Y713-L727, G714-L727, S715-L727, G716-L727, Y717-L727, L718-L727, L719-L727, A720-L727, and/or S721-L727 of SEQ ID NO: 1. Polynucleotide sequences encoding these polypeptides are also provided. The present invention also encompasses the use of these N-terminal HNTTBMY1 deletion polypeptides as immunogenic and/or antigenic epitopes as described elsewhere herein.

In preferred embodiments, the following C-terminal HNTTBMY1 deletion polypeptides are encompassed by the present invention: M1-L727, M1-E726, M1-S725, M1-E724, M1-P723, M1-T722, M1-S721, M1-A720, M1-L719, M1-L718, M1-Y717, M1-G716, M1-S715, M1-G714, M1-Y713, M1-R712, M1-G711, M1-N710, M1-P709, M1-N708, M1-G707, M1-S706, M1-T705, M1-E704, M1-L703, M1-P702, M1-S701, M1-T700, M1-S699, M1-G698, M1-P697, M1-G696, M1-L695, M1-Y694, M1-S693, M1-R692, M1-H691, M1-T690, M1-P689, M1-M688, M1-P687, M1-S686, M1-P685, M1-A684, M1-E683, M1-S682, M1-P681, M1-V680, M1-K679, M1-S678, M1-L677, M1-I676, M1-F675, M1-R674, M1-T673, M1-E672, M1-D671, M1-N670, M1-E669, M1-E668, M1-L667, M1-N666, M1-S665, M1-I664, M1-D663, M1-K662, M1-M661, M1-M660, M1-R659, M1-G658, M1-K657, M1-K656, M1-Y655, M1-S654, M1-V653, M1-S652, M1-L651, M1-T650, M1-N649, M1-S648, MI-G647, M1-D646, M1-S645, M1-L644, M1-L643, M1-H642, M1-F641, M1-H 640, M1-R639, M1-L638, M1-V637, M1-F636, M1-V635, M1-V634, M1-P633, M1-I632, M1-P631, M1-L630, M1-T629, M1-A628, M1-V627, M1-V626, M1-I625, M1-L624, M1-T623, M1-I622, M1-L621, M1-L620, M1-A619, M1-M618, M1-A617, M1-W616, M1-N615, M1-P614, M1-F613, M1-Y612, M1-L611, M1-Y610, M1-R609, M1-E608, M1-A607, M1-A606, M1-E605, M1-E604, M1-K603, M1-I602, M1-W601, M1-A600, M1-S599, M1-Y598, M1-G597, M1-P596, M1-P595, M1-T594, M1-V593, M1-G592, M1-L591, M1-Q590, M1-I589, M1-I588, M1-S587, M1-A586, M1-T585, M1-T584, M1-L583, M1-V582, M1-A581, M1-M580, M1-C579, M1-L578, M1-P577, M1-S576, M1-V575, M1-F574, M1-K573, M1-W572, M1-M571, M1-Y570, M1-F569, M1-Y568, M1-F567, M1-R566, M1-Y565, M1-P564, M1-R563, M1-F562, M1-G561, M1-L560, M1-M559, M1-E558, M1-T557, M1-L556, M1-E555, M1-Q554, M1-M553, M1-F552, M1-K551, M1-K550, M1-T549, M1-G548, M1-Y547, M1-I546, M1-W545, MI-A544, M1-V543, M1-A542, M1-I541, M1-N540, M1-E539, M1-L538, M1-I537, M1-V536, M1-I535, M1-L534, M1-T533, M1-L532, M1-P531, M1-L530, M1-T529, M1-A528, M1-S527, M1-Y526, M1-D525, M1-D524, M1-F523, M1-M522, M1-T521, M1-V520, M1-F519, M1-Y518, M1-N517, M1-G516, M1-S515, M1-R514, M1-Q513, M1-V512, M1-F511, M1-L510, M1-L509, M1-G508, M1-V507, M1-L506, M1-F505, M1-A504, M1-F503, M1-V502, M1-C501, M1-C500, M1-G499, M1-V498, M1-T497, M1-F496, M1-M495, M1-E494, M1-K493, M1-P492, M1-V491, M1-K490, M1-F489, M1-T488, M1-D487, M1-I486, M1-I485, M1-P484, M1-T483, M1-T482, M1-I481, M1-G480, M1-A479, M1-M478, M1-T477, M1-G476, M1-I475, M1-M474, M1-S473, M1-G472, M1-L471, M1-G470, M1-L469, M1-N468, M1-I467, M1-L466, M1-M465, M1-L464, M1-F463, M1-F462, M1-M461, M1-V460, M1-S459, M1-W458, M1-F457, M1-P456, M1-S455, M1-A454, M1-P453, M1-F452, M1-H451, M1-T450, M1-M449, M1-A448, M1-E447, M1-T446, MI-F445, M1-A444, M1-I443, M1-F442, M1-A441, M1-L440, M1-G439, M1-T438, M1-G437, M1-Q436, M1-V435, M1-S434, M1-K433, M1-D432, M1-L431, M1-E430, M1-D429, M1-E428, M1-L427, M1-L426, M1-C425, M1-P424, M1-D423, M1-L422, M1-G421, M1-L420, M1-A419, M1-S418, M1-F417, M1-Q416, M1-D415, M1-E414, M1-K413, M1-V412, M1-T411, M1-M410, M1-I409, M1-V408, M1-N407, M1-Y406, M1-M405, M1-E404, M1-M403, M1-Y402, M1-D401, M1-K400, M1-T399, M1-T398, M1-L397, M1-H396, M1-S395, M1-F394, M1-N393, M1-V392, M1-H391, M1-P390, M1-P389, M1-I388, M1-L387, M1-D386, M1-R385, M1-S384, M1-L383, M1-V382, M1-N381, M1-T380, M1-N379, M1-L378, M1-Y377, M1-G376, M1-L375, M1-I374, M1-K373, M1-E372, M1-A371, M1-N370, M1-E369, M1-V368, M1-V367, M1-C366, M1-K365, M1-E364, M1-N363, M1-M362, M1-I361, M1-N360, M1-A359, M1-K358, M1-F357, M1-G356, M1-L355, M1-V354, M1-A353, M1-F352, M1-V351, M1-V350, M1-L349, M1-T348, M1-A347, M1-L346, M1-V345, M1-S344, M1-T343, M1-F342, M1-F341, M1-N340, M1-I339, M1-F338, M1-S337, M1-V336, M1-L335, M1-A334, M1-A333, M1-D332, M1-F331, M1-H330, M1-C329, M1-N328, M1-N327, M1-D326, M1-Q325, M1-K324, M1-N323, M1-Y322, M1-S321, M1-S320, M1-F319, M1-A318, M1-1317, M1-V316, M1-G315, M1-G314, M1-F313, M1-G312, M1-L311, M1-G310, M1-L309, M1-A308, M1-F307, M1-F306, M1-V305, M1-Q304, M1-T303, M1-A302, M1-A301, M1-E300, M1-R299, M1-W298, M1-V297, M1-Q296, M1-P295, M1-D294, M1-L293, M1-M292, M1-K291, M1-D290, M1-L289, M1-K288, M1-P287, M1-T286, M1-F285, M1-M284, M1-H283, M1-L282, M1-I281, M1-G280, Ml-D279, M1-V278, M1-A277, M1-G276, M1-R275, M1-L274, M1-L273, M1-L272, M1-G271, M1-R270, M1-V269, M1-L268, M1-F267, M1-C266, M1-A265, M1-L264, M1-V263, M1-V262, M1-Y261, M1-P260, M1-F259, M1-L258, M1-S257, M1-S256, M1-F255, M1-Y254, M1-M253, M1-V252, M1-K251, M1-G250, M1-S249, M1-S248, M1-Q247, M1-I246, M1-G245, M1-K244, M1-V243, M1-V242, M1-A241, M1-M240, M1-G239, M1-V238, M1-I237, M1-S236, M1-W235, M1-A234, M1-V233, M1-L232, M1-L231, M1-C230, M1-L229, M1-T228, M1-M227, M1-K226, M1-W225, M1-N224, M1-L223, M1-G222, M1-G221, M1-S220, M1-E219, M1-S218, M1-I217, M1-S216, M1-D215, M1-S214, M1-I213, M1-D212, M1-L211, M1-A210, M1-E209, M1-R208, M1-Y207, M1-W206, M1-F205, M1-Y204, M1-T203, M1-T202, M1-A201, M1-S200, M1-S199, M1-K198, M1-E197, M1-C196, M1-E195, M1-A194, M1-E193, M1-V192, M1-V191, M1-A190, M1-V189, M1-S188, M1-G187, M1-N186, M1-R185, M1-V184, M1-V183, M1-P182, M1-C181, M1-E180, M1-S179, M1-W178, M1-P177, M1-L176, M1-P175, M1-Y174, M1-Q173, M1-F172, M1-S171, M1-K170, M1-F169, M1-F168, M1-Y167, M1-F166, M1-I165, M1-S164, M1-W163, M1-G162, M1-I161, M1-I160, M1-V159, M1-N158, M1-Y157, M1-Y156, M1-L155, M1-G154, M1-V 153, M1-F152, M1-L151, M1-C150, M1-V149, M1-I148, M1-C147, M1-S146, M1-S145, M1-F144, M1-G143, M1-I142, M1-G141, M1-G140, M1-L139, M1-R138, M1-P137, M1-C136, M1-I135, M1-Y134, M1-H133, M1-W132, M1-V131, M1-G130, M1-I129, M1-S128, M1-G127, M1-R126, M1-R125, M1-I124, M1-R123, M1-Q122, M1-G121, M1-V120, M1-A119, M1-L118, M1-E117, M1-L116, M1-F115, M1-F114, M1-L113, M1-P112, M1-I111, M1-G110, M1-I109, M1-I108, M1-I107, M1-L106, M1-L105, M1-V104, M1-L103, M1-Y102, M1-P101, M1-V100, M1-L99, M1-Y98, M1-A97, M1-G96, M1-G95, M1-G94, M1-N93, M1-K92, M1-Q91, C90, M1-L89, M1-Y88, M1-P87, M1-F86, M1-R85, M1-W84, M1-I83, M1-N82, M1-G81, M1-L80, M1-G79, M1-V78, M1-S77, M1-F76, M1-G75, M1-I74, M1-Q73, M1-A72, M1-L71, M1-I70, M1-Y69, M1-Q68, M1-L67, M1-K66, M1-S65, M1-N64, M1-W63, M1-A62, M1-P61, M1-R60, M1-D59, M1-E58, M1-T57, M1-D56, M1-L55, M1-E54, M1-E53, M1-E52, M1-V51, M1-A50, M1-K49, M1-Q48, M1-K47, M1-G46, M1-G45, M1-A44, M1-E43, M1-G42, M1-A41, M1-V40, M1-N39, M1-L38, M1-V37, M1-S36, M1-Q35, M1-K34, M1-Y33, M1-D32, M1-V31, M1-P30, M1-E29, M1-E28, M1-L27, M1-A26, M1-L25, M1-L24, M1-D23, M1-A22, M1-V21, M1-S20, M1-E19, M1-T18, M1-V17, M1-H16, M1-E15, M1-S14, M1-S13, M1-H12, M1-E11, M1-R10, M1-Q9, M1-T8, and/or M1-V7 of SEQ ID NO: 1. Polynucleotide sequences encoding these polypeptides are also provided. The present invention also encompasses the use of these C-terminal HNTTBMY1 deletion polypeptides as immunogenic and/or antigenic epitopes as described elsewhere herein.

Alternatively, preferred polypeptides of the present invention may comprise polypeptide sequences corresponding to, for example, internal regions of the HNTTBMY1 polypeptide (e.g., any combination of both N- and C-terminal HNTTBMY1 polypeptide deletions) of SEQ ID NO: 1. For example, internal regions could be defined by the equation: amino acid NX to amino acid CX, wherein NX refers to any N-terminal deletion polypeptide amino acid of HNTTBMY1 (SEQ ID NO: 1), and where CX refers to any C-terminal deletion polypeptide amino acid of HNTTBMY1 (SEQ ID NO: 1). Polynucleotides encoding these polypeptides are also provided. The present invention also encompasses the use of these polypeptides as an immunogenic and/or antigenic epitope as described elsewhere herein.

### Example 32

Method of Assessing the Level of Neurotransmitter Receptor Activity of the HNTTBMY1 Polypeptide of the Present Invention

Assays for characterizing the level of neurotransmitter receptor activity of the HNTTBMY1 polypeptide of the present invention are well known and routine in the art. For example, such activity may be addressed by measuring the uptake level of known radiolabeled neurotransmitter receptor substrates, such as dopamine, choline, GABA, glycine, glutamate, aspartate, taurine, tyrosine, adenosine, histamine, among others, in cells such as COS-7 (green monkey kidney cells) transfected with an expression vector comprising the HNTTBMY1 transporter of the present invention and comparing this level to non-transfected cells (Shimada et al. 1991). Assays in 24-well plates (2×10\hat{ }5 cells per well) at 37 degrees Celsius for measuring the level of uptake in 10 minutes in the presence of various radioactive compounds have also been described (Giros et al. 1992).

Appropriate controls for such experiments would need to be implemented to ensure the expressed HNTTBMY1 polypeptide is localized to the cell membrane. Such a control could be constructed by adding a FLAG tag epitope to the coding region of the HNTTBMY1 polypeptide using methods well known in the art. Upon transfecting COS-7 cells, for example, antibodies directed against the FLAG tag epitope and fused to an appropriate label or reporter (e.g., GFP, fluorescent dyes, etc,) may be used to assess whether the protein is localized to the cell membrane. In the instance where the HNTTBMY1 polypeptide does not localize to the cell membrane, routine methods may be employed to delete any internalization signals in the amino acid sequence and the experiment can then be repeated.

The HNTTBMY1-FLAG tag vector may be constructed according to the following method. The putative neurotransmitter receptor HNTTBMY1cDNA may be PCR amplified using PFU™ (Stratagene). The primers used in the PCR reaction are specific to the HNTTBMY1polynucleotide and may be ordered from Gibco BRL (5 prime primer: 5′-GTCCCCAAGCTTGCACCATGCCGAAGAACAGCAAAGTGACCC-3′ (SEQ ID NO: 57), 3 prime primer: 5′-CGGGATCCTACAGCTCCGACTCAGGGG-3′ (SEQ ID NO: 59), wherein the 5 prime primer contains a HindIII site at the 5′ end, and the 3 prime primer contains a BamHI site at the 5′ end and an optimal Kozak sequence. The following 3 prime primer may be used to add a Flag-tag epitope to the HNTTBMY1 polypeptide for immunocytochemistry: 5′-CGGGATCCTACTTGTCGTCGTCGTCCTTGTAGTCCAGCTCCGACTCAGGGG-3′ (SEQ ID NO: 58), wherein the primer contains a BamHI site at the 5′ end, an optimal Kozak sequence, in addition to a sequence encoding the FLAG tag epitope. The product from the PCR reaction may be isolated from a 0.8% Agarose gel (Invitrogen) and purified using a Gel Extraction Kit™ from Qiagen.

The purified product may be then digested overnight along with the pcDNA3.1 Hygro™ mammalian expression vector from Invitrogen using the HindIII and BamHI restriction enzymes (New England Biolabs). These digested products are then purified using the Gel Extraction Kit™ from Qiagen and subsequently ligated to the pcDNA3.1 Hygro™ expression vector using a DNA molar ratio of 4 parts insert: 1 vector. All DNA modification enzymes are purchased from NEB. The ligation may be incubated overnight at 16 degrees Celsius, after which time, one microliter of the mix may be used to transform DH5 alpha cloning efficiency competent E. coli™ (Gibco BRL). A detailed description of the pcDNA3.1 Hygro™ mammalian expression vector is available at the Invitrogen web site (www.Invitrogen.com). The plasmid DNA from the ampicillin resistant clones are isolated using the Wizard DNA Miniprep System™ from Promega. Positive clones are then confirmed and scaled up for purification using the Qiagen Maxiprep™ plasmid DNA purification kit.

Once the HNTTBMY1 expression vector is obtained, it may be used to transfect COS cells, among others, using Lipofectamine 2000™ according to the manufacturers specifications (Gibco BRL). Two days later, the cells may be split 1:3 into selective media (DMEM 11056, 600 ug/ml Hygromycin, 200 ug/ml Zeocin, 10% FBS). All cell culture reagents can be purchased from Gibco BRL-Invitrogen.

The transfected cells are then used to assess the HNTTBMY1 polypeptides cell surface localization via immunohistochemistry. The cell lines transfected and selected for expression of Flag-epitope tagged orphan GPCRs are analyzed by immunocytochemistry. The cells are plated at 1×10\hat{ }3 in each well of a glass slide (VWR). The cells are rinsed with PBS followed by acid fixation for 30 minutes at room temperature using a mixture of 5% Glacial Acetic Acid/90% ETOH. The cells are then blocked in 2% BSA and 0.1% Triton in PBS, incubated for 2 h at room temperature or overnight at 4(C. A monoclonal anti-Flag FITC antibody may be diluted at 1:50 in blocking solution and incubated with the cells for 2 h at room temperature. Cells are then may be shed three times with 0.1% Triton in PBS for five minutes. The slides are overlayed with mounting media dropwise with Biomedia-Gel Mount™ (Biomedia; Containing Anti-Quenching Agent). Cells are examined at 10× magnification using a Nikon TE300 equipped with an FITC filter (535nm).

References

Shimada S, Kitayama S, Lin C L, Patel E, Nanthakumar E, Gregor P, Kuhar M and Uhl G. Cloning and expression of a cocaine-sensitive dopamine transporter complementary DNA. Science, 254. 576-578 (1991)

Giros B, Mestikawy S, Godinot N, Yang-Feng T, and Caron M G. Cloning, pharmacological characterization and chromosome assignment of the human dopamine transporter. Mol. Pharmacol. 42, 383-390 (1992)

One skilled in the art could easily modify the exemplified studies to test the activity of polynucleotides of the invention (e.g., gene therapy), agonists, and/or antagonists of polynucleotides or polypeptides of the invention.

It will be clear that the invention may be practiced otherwise than as particularly described in the foregoing description and examples. Numerous modifications and variations of the present invention are possible in light of the above teachings and, therefore, are within the scope of the appended claims.

The entire, disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

What is claimed is:

1. An isolated nucleic acid molecule comprising a polynucleotide having a nucleotide sequence selected from the group consisting of:
(a) a polynucleotide fragment of SEQ ID NO: 1 or a polynucleotide fragment of the cDNA sequence included in ATCC Deposit No: PTA-4803, which is hybridizable to SEQ ID NO: 1;
(b) a polynucleotide encoding a polypeptide fragment of SEQ ID NO: 2 or a polypeptide fragment encoded by the cDNA sequence included in ATCC Deposit No: PTA-4803, which is hybridizable to SEQ ID NO: 1;
(c) a polynucleotide encoding a polypeptide domain of SEQ ID NO: 2 or a polypeptide domain encoded by the cDNA sequence included in ATCC Deposit No: PTA-4803, which is hybridizable to SEQ ID NO: 1;
(d) a polynucleotide encoding a polypeptide epitope of SEQ ID NO: 2 or a polypeptide epitope encoded by the cDNA sequence included in ATCC Deposit No: PTA-4803, which is hybridizable to SEQ ID NO: 1;
(e) a polynucleotide encoding a polypeptide of SEQ ID NO: 2 or the cDNA sequence included in ATCC Deposit No: PTA-4803, which is hybridizable to SEQ ID NO: 1, having neurotransmitter transporter activity;
(f) an isolated polynucleotide comprising nucleotides 383 to 2569 of SEQ ID NO: 1, wherein said nucleotides encode a polypeptide corresponding to amino acids 2 to 727 of SEQ ID NO: 2 minus the start codon;
(g) an isolated polynucleotide comprising nucleotides 380 to 2569 of SEQ ID NO: 1, wherein said nucleotides encode a polypeptide corresponding to amino acids 1 to 727 of SEQ ID NO: 2 including the start codon;
(h) a polynucleotide which represents the complimentary sequence (antisense) of SEQ ID NO: 1; and
(i) a polynucleotide capable of hybridizing under stringent conditions to any one of the polynucleotides specified in (a)-(h), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment consists of a nucleotide sequence encoding a human neurotransmitter transporter.

3. A recombinant vector comprising the isolated nucleic acid molecule of claim 1.

4. A recombinant host cell comprising the vector sequences of claim 3.

5. An isolated polypeptide comprising an amino acid sequence selected from the group consisting of:
(a) a polypeptide fragment of SEQ ID NO: 2 or the encoded sequence included in ATCC Deposit No: PTA-4803;
(b) a polypeptide fragment of SEQ ID NO: 2 or the encoded sequence included in ATCC Deposit No: PTA-4803, having neurotransmitter transporter activity;
(c) a polypeptide domain of SEQ ID NO: 2 or the encoded sequence included in ATCC Deposit No: PTA-4803;
(d) a polypeptide epitope of SEQ ID NO: 2 or the encoded sequence included in ATCC Deposit No: PTA-4803;
(e) a full length protein of SEQ ID NO: 2 or the encoded sequence included in ATCC Deposit No: PTA-4803;
(f) a polypeptide comprising amino acids 2 to 727 of SEQ ID NO: 2, wherein said amino acids 2 to 727 comprising a polypeptide of SEQ ID NO: 2 minus the start methionine; and
(g) a polypeptide comprising amino acids 1 to 727 of SEQ ID NO: 2.

6. The isolated polypeptide of claim 5, wherein the full length protein comprises sequential amino acid deletions from either the C-terminus or the N-terminus.

7. An isolated antibody that binds specifically to the isolated polypeptide of claim 5.

8. A recombinant host cell that expresses the isolated polypeptide of claim 5.

9. A method of making an isolated polypeptide comprising:
(a) culturing the recombinant host cell of claim 8 under conditions such that said polypeptide is expressed; and
(b) recovering said polypeptide.

10. The polypeptide produced by claim 9.

11. A method for preventing, treating, or ameliorating a medical condition, comprising the step of administering to a mammalian subject a therapeutically effective amount of the polypeptide of claim 5, or a modulator thereof.

12. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

13. A method of diagnosing a pathological condition or a susceptibility to a pathological condition in a subject comprising:
(a) determining the presence or amount of expression of the polypeptide of claim 5 in a biological sample; and
(b) diagnosing a pathological condition or a susceptibility to a pathological condition based on the presence or amount of expression of the polypeptide.

14. An isolated nucleic acid molecule consisting of a polynucleotide having a nucleotide sequence selected from the group consisting of:
(a) a polynucleotide encoding a polypeptide of SEQ ID NO: 2;
(b) an isolated polynucleotide consisting of nucleotides 383 to 2569 of SEQ ID NO: 1, wherein said nucleotides encode a polypeptide corresponding to amino acids 2 to 727 of SEQ ID NO: 2 minus the start codon;
(c) an isolated polynucleotide consisting of nucleotides 380 to 2569 of SEQ ID NO: 1, wherein said nucleotides encode a polypeptide corresponding to amino acids 1 to 727 of SEQ ID NO: 2 including the start codon;
(d) a polynucleotide encoding the HNTTBMY1 polypeptide encoded by the cDNA clone contained in ATCC Deposit No. PTA-4803; and
(e) a polynucleotide which represents the complimentary sequence (antisense) of SEQ ID NO: 1.

15. The isolated nucleic acid molecule of claim 14, wherein the polynucleotide comprises a nucleotide sequence encoding a human G-protein coupled receptor.

16. A recombinant vector comprising the isolated nucleic acid molecule of claim 15.

17. A recombinant host cell comprising the recombinant vector of claim 16.

18. An isolated polypeptide consisting of an amino acid sequence selected from the group consisting of:
(a) a polypeptide fragment of SEQ ID NO: 2 having neurotransmitter transporter activity;
(b) a polypeptide domain of SEQ ID NO: 2 having neurotransmitter transporter activity;
(c) a full length protein of SEQ ID NO: 2;
(d) a polypeptide corresponding to amino acids 2 to 727 of SEQ ID NO: 2, wherein said amino acids 2 to 727 consisting of a polypeptide of SEQ ID NO: 2 minus the start methionine;
(e) a polypeptide corresponding to amino acids 1 to 727 of SEQ ID NO: 2; and
(f) a polypeptide encoded by the cDNA contained in ATCC Deposit No. PTA-4803.

19. The method of diagnosing a pathological condition of claim 15 wherein the condition is a member of the group consisting of: a disorder related to aberrant neurotransmitter transport; affective disorders, psychotic disorders, neurological disorders; metabolic disorders, immune-related disorders, hypotension, hypertension, endocrinal diseases, growth disorders, neuropathic pain, obesity, anorexia, bulimia, Parkinson's disease, dementias, behavioral disorder; memory disorders; cognitive disorders; disorders associated with aberrant serotonin expression and/or activity; anxiety, fear, depression, sleep, pain, disorders associated with aberrant maintenance of an attentive or alert state; attention deficit disorders; disorders affecting the ‘reward center’ of the brain; disorders affecting the synthesis, and/or effecting the release of neurotransmitters such as dopamine, opioid peptides, serotonin, GABA, and glutamate; addictive disorders; homeostatic disorders; neuroendocrine disorders; disorders affecting the establishment of long term potentiation; circadian rhythm disorders; disorders associated with the establishment of aberrant sleep/wake cycles; dopaminergic functional disorders; neuronal transmission system disorders, and pain.

20. The method for preventing, treating, or ameliorating a medical condition of claim 11, wherein the medical condition is selected from the group consisting of: a disorder related to aberrant neurotransmitter transport; affective disorders, psychotic disorders, neurological disorders; metabolic disorders, immune-related disorders, hypotension, hypertension, endocrinal diseases, growth disorders, neuropathic pain, obesity, anorexia, bulimia, Parkinson's disease, dementias, behavioral disorder; memory disorders; cognitive disorders; disorders associated with aberrant serotonin expression and/or activity; anxiety, fear, depression, sleep, pain, disorders associated with aberrant maintenance of an attentive or alert state; attention deficit disorders; disorders affecting the ‘reward center’ of the brain; disorders affecting the synthesis, and/or effecting the release of neurotransmitters such as dopamine, opioid peptides, serotonin, GABA, and glutamate; addictive disorders; homeostatic disorders; neuroendocrine disorders; disorders affecting the establishment of long term potentiation; circadian rhythm disorders; disorders associated with the establishment of aberrant sleep/wake cycles; dopaminergic functional disorders; neuronal transmission system disorders, and pain.


SEQUENCE LISTING (135 sequences)

SEQ ID NO: 1 - protein, 727 aa, Homo sapiens [sequence omitted]

SEQ ID NO: 2 - DNA, 2572 bp, Homo sapiens
cggcactcggccccagtcttcgcaatcccgcgcgctctctccgccgtgggacctgggtccccgcgccgctcccctgagccccgagcgcgcggcgggcgggagccaggttgggactggtggtgaggcagggagtgaggagcgagcggagtcgcgtgcgccggcgcgcagctccgggtcgccccagccccagccgggggcctgtggcccgggggaggagctgtgcgtccgcgacccgtcggggatcgcagctgctcggccggagtgcacgggccgagtctgcgcgactacccacgcgtgacaggtccctgaatgagaaggagctgacagcagctgaattccatcttctctgtgtgctggggagcagggctacacggcccaggtggcatcaatgccgaagaacagcaaagtgacccagcgtgagcacagcagtgagcatgtcactgagtccgtggccgacctgctggccctcgaggagcctgtggactataagcagagtgtactgaatgtggctggtgaggcaggcggcaagcagaaggcggtggaggaggagctggacacagaggaccggccggcctggaacagtaagctgcagtacatcctggcccagattggcttctctgtgggcctcggcaacatctggaggttcccctacctgtgccagaaaaatggaggaggtgcttacctggtgccctacctggtgctgctgatcatcatcgggatccccctcttcttcctggagctggctgtgggtcagaggatccgccgcggcagcatcggtgtgtggcactatatatgtccccgcctggggggcatcggcttctccagctgcatagtctgtctctttgtggggctgtattataatgtgatcatcgggtggagcatcttctatttcttcaagtccttccagtacccgctgccctggagtgaatgtcctgtcgtcaggaatgggagcgtggcagtggtggaggcagagtgtgaaaagagctcagccactacctacttctggtaccgagaggccttggacatctctgactccatctcggagagtgggggcctcaactggaagatgaccctgtgcctcctcgtggcctggagcatcgtggggatggctgtcgttaagggcatccagtcctcggggaaggtgatgtatttcagctccctcttcccctacgtggtgctggcctgcttcctggtccgggggctgttgctgcgaggggcagttgatggcatcctacacatgttcactcccaagctggacaagatgctggacccccaggtgtggcgggaggcagctacccaggtcttctttgccttgggcctgggctttggtggtgtcattgccttctccagctacaataagcaggacaacaactgccacttcgatgccgccctggtgtccttcatcaacttcttcacgtcagtgttggccaccctcgtggtgtttgctgtgctgggcttcaaggccaacatcatgaatgagaagtgtgtggtcgagaatgctgagaaaatcctagggtaccttaacaccaacgtcctgagccgggacctcatcccaccccacgtcaacttctcccacctgaccacaaaggactacatggagatgtacaatgtcatcatgaccgtgaaggaggaccagttctcagccctgggccttgacccctgccttctggaggacgagctggacaagtccgtgcagggcacaggcctggccttcatcgccttcactgaggccatgacgcacttccccgcctccccgttctggtccgtcatgttcttcttgatgcttatcaacctgggcctgggcagcatgatcgggaccatggcaggcatcaccacgcccatcatcgacaccttcaaggtgcccaaggagatgttcacagtgggctgctgtgtctttgcattcctcgtggggctgttgttcgtccagcgctccggaaactactttgtcaccatgttcgatgactactcggccaccctgccactcactctcatcgtcatccttgagaacatcgctgtggcctggatttatggaaccaagaagttcatgcaggagctgacggagatgctgggcttccgcccctaccgcttctatttctacatgtggaagttcgtgtctccactatgcatggctgtgctcaccacagccagcatcatccagctgggggtcacgcccccgggctacagcgcctggatcaaggaggaggctgccgagcgctacctgtatttccccaactgggccatggcactcctgatcaccctcatcgtcgtggcgacgctgcccatccctgtggtgttcgtcctgcggcacttccacctgctctctgatggctccaacaccctctccgtgtcctacaagaagggccgcatgatgaaggacatctccaacctggaggagaacgatgagacccgcttcatcctcagcaaggtgcccagtgaggcaccttcccccatgcccactcaccgttcctatctggggcccggcagcacatcacccctggagaccagcggtaaccccaatggacgctatgggagcggctacctcctggccagcacccctgagtcggagctgtga

SEQ ID NO: 3 - DNA, 388 bp, Homo sapiens
cggcactcggccccagtcttcgcaatcccgcgcgctctctccgccgtgggacctgggtccccgcgccgctcccctgagccccgagcgcgcggcgggcgggagccaggttgggactggtggtgaggcagggagtgaggagcgagcggagtcgcgtgcgccggcgcgcagctccgggtcgccccagccccagccgggggcctgtggcccgggggaggagctgtgcgtccgcgacccgtcggggatcgcagctgctcggccggagtgcacgggccgagtctgcgcgactacccacgcgtgacaggtccctgaatgagaaggagctgacagcagctgaattccatcttctctgtgtgctggggagcagggctacacggcccaggtggcatca

SEQ ID NO: 4 - DNA, 80 bp, Homo sapiens
ttgagaacatcgctgtggcctggatttatggaaccaagaagttcatgcaggagctgacggagatgctgggcttccgcccc

SEQ ID NO: 5 - DNA, 80 bp, Homo sapiens
ggactacatggagatgtacaatgtcatcatgaccgtgaaggaggaccagttctcagccctgggccttgacccctgccttc

SEQ ID NO: 6 - DNA, 20 bp, bacteriophage T7
taatacgactcactataggg

SEQ ID NO: 7 - DNA, 18 bp, bacteriophage T7
atttaggtgacactatag

SEQ ID NO: 8 - DNA, 20 bp, Homo sapiens
gacttgtccagctcgtcctc

SEQ ID NO: 9 - DNA, 20 bp, Homo sapiens
tcaacttctcccacctgacc

SEQ ID NO: 10 - DNA, 20 bp, Homo sapiens
gccactcactctcatcgtca

SEQ ID NO: 11 - DNA, 20 bp, Homo sapiens
ttggggaaatacaggtagcg

SEQ ID NO: 12 - protein, 225 aa, Bos taurus
NVWRFPYLCQKNGGGAYLVPYLVLLIIIGIPLFFLELAVGQRIRRGSIGVWHYVCPRLGGIGFSSCIVCLFVGLYYNVIIGWSIFYFFKSFQYPLPWSECPVSRNGTVAVVEAECEKSSATTYFWYREALDISNSISESGGLNWKMTLCLLVAWRIVGMAVVKGIQSSGKVMYFSSLFPYVVLACFLVRGLLLRGAIDGILHMFTPKLDKMLDPQVWRDAATQIF

SEQ ID NO: 13 - protein, 727 aa, Rattus norvegicus
MPKNSKVTQREHSNEHVTESVADLLALEEPVDYKQSVLNVAGETGGKQKVAEEELDAEDRPAWNSKLQYILAQIGFSVGLGNIWRFPYLCQKNGGGAYLVPYLVLLIIIGIPLFFLELAVGQRIRRGSIGVWHYVCPRLGGIGFSSCIVCLFVGLYYNVIIGWSVFYFFKSFQYPLPWSECPVIRNGTVAVVEPECEKSSATTYFWYREALDISNSISESGGLNWKMTVCLLVAWSIVGMAVVKGIQSSGKVMYFSSLFPYVVLACFLVRGLLLRGAVDGILHMFTPKLDKMLDPQVWREAATQVFFALGLGFGGVIAFSSYNKQDNNCHFDAALVSFINFFTSVLATLVVFAVLGFKANIMNEKCVVENAEKILGYLNSNVLSRDLIPPHVNFSHLTTKDYSEMYNVIMTVKEKQFSALGLDPCLLEDELDKSVQGTGLAFIAFTEAMTHFPASPFWSVMFFLMLINLGLGSMIGTMAGITTPIIDTFKVPKEMFTVGCCVFAFFVGLLFVQRSGNYFVTMFDDYSATLPLTVIVILENIAVAWIYGTKKFMQELTEMLGFRPYRFYFYMWKFVSPLCMAVLTTASIIQLGVSPPGYSAWIKEEAAERYLYFPNWAMALLITLIAVATLPIPVVFILRHFHLLSDGSNTLSVSYKKGRMMKDISNLEENDETRFILSKVPSEAPSPMPTHRSYLGPGSTSPLESSSHPNGRYGSGYLLASTPESEL

SEQ ID NO: 14 - protein, 729 aa, Rattus norvegicus
MPKNSKVVKRDLDDDVIESVKDLLSNEDSVEDVSKKSELIVDVQEEKDTDAEDGSEVDDERPAWNSKLQYILAQVGFSVGLGNVWRFPYLCQKNGGGAYLLPYLILLLVIGIPLFFLELSVGQRIRRGSIGVWNYISPKLGGIGFASCVVCYFVALYYNVIIGWTLFYFSQSFQQPLPWDQCPLVKNASHTYIEPECEKSSATTYYWYREALAISSSISESGGLNWKMTGCLLAAWVMVCLAMIKGIQSSGKIMYFSSLFPYVVLICFLIRSLLLNGSIDGIRHMFTPKLEMMLEPKVWREAATQVFFALGLGFGGVIAFSSYNKRDNNCHFDAVLVSFINFFTSVLATLVVFAVLGFKANIVNEKCISQNSEMILKLLKTGNVSWDVIPRHINLSAVTAEDYHVVYDIIQKVKEEEFAVLHLKACQIEDELNKAVQGTGLAFIAFTEAMTHFPASPFWSVMFFLMLINLGLGSMFGTIEGIITPVVDTFKVRKEILTVICCLLAFCIGLMFVQRSGNYFVTMFDDYSATLPLLIVVILENIAVSFVYGIDKFLEDLTDMLGFAPSKYYYYMWKYISPLMLVTLLIASIVNMGLSPPGYNAWIKEKASEEFLSYPMWGMVVCFSLMVLAILPVPVVFVIRRCNLIDDSSGNLASVTYKRGRVLKEPVNLDGDDASLIHGKIPSEMSSPNFGKNIYRKQSGSPTLDTAPNGRYGIGYLMADMPDMPESDL

SEQ ID NO: 15 - protein, 316 aa, Homo sapiens
  MISC_FEATURE (253)..(253): wherein “X” equals any amino acid.
MEKARPLWANSLQFVFACISYAVGLGNVWRNPYLCQMYGGGSFLVPYIIMLIVEGMPLLYLELAVGQRMRQGSIGAWRTISPYLSGVGVASVVVSFFLSMYYNVINAWAFWYLFHSFQDPLPWSVCPLNGNHTGYDEECEKASSTQYFWYRKTLNISPSLQENGGVQWEPALCLLLAWLVVYLCILRGTESTGKVVYFTASLPYCVLIIYLIRGLTVHGATNGLMYMFTPKIEQLANPKAWINAATQIFFSLXLGFGSLIAFASYNEPSNNCQKHAIIVSLINSFTSIFASIVTFSIYGFKATFNYENCLKKVSLX

SEQ ID NO: 16 - protein, 635 aa, Mus musculus
  MISC_FEATURE (114)..(114): wherein “X” equals any amino acid.
MESPSAHAVSLPEDEELQPWGGAGGPGQHPGRPRSTECAHPGVVEKVRPKWDNPLQFLLVCISYAVGLGNVWRFPYLCQMYGGGSFLVPYIIMLIVEGMPLLYLELAVGQRMRXGSIGAWRTISPYLSGVGIASLVVSFLASVYFNVINTWALWYLFHSFQDPLPWSVCPLNSNHTGYDEECEKASSTQYFWYRKTLNISPSIQENGGVQWEPALCLTLAWLMVYLCILRGTESTGKVVYFTTSLPYFVLIIYLVRGLTLHGATNGLAYMFTPKIEQLANPKAWINAATQIFFSLGLGCGGLIAFASYNEPSNDCQKHALIVSVINSTTAIFSSIVTFSIYGFKATFNYENCLNKVILLLTNSFDLEDGFLTVSNLEEVKNYLASTYPNKYSEVFPHIRNCSLESELDTAVQGTGLAFIVYTEAIKNMEVSQLWSVLYFFMLLTLGMGSMVGTGTAILTPLTDSKIISSYLPKEAISGLVCLLNCAIGMVFTMEAGNYWFDLFNDYTATLSLLLIVLVETIAVCYVYGLKRFESDLRAMTGRTLSWYWKVMWAFVSPLLIVGLFIFYLSDYILTGTLQYQAWDATQGHVVTKDYPTYALAVIGLLVASSTMCIPLVALGTFVTRHFKIREQFSAA

SEQ ID NO: 17 - protein, 615 aa, Mus musculus [sequence omitted]

SEQ ID NO: 18 - protein, 605 aa, Mus musculus
MAQASGMDPLVDIEDERPKWDNKLQYLLSCIGFAVGLGNIWRFPYLCQTHGGGAFLIPYFIALVFEGIPLFYIELAIGQRLRRGSIGVWKTISPYLGGVGLGCFSVSFLVSLYYNTVLLWVLWFFLNSFQHPLPWSTCPLDLNRTGFVQECQSSGTVSYFWYRQTLNITSDISNTGTIQWKLFLCLVACWSTVYLCVIRGIESTGKVIYFTALFPYLVLTIFLIRGLTLPGATEGLIYLFTPNMKTLQNPRVWLDAATQIFFSLSLAFGGHIAFASYNPPRNNCEKDAVIIALVNSMTSLYASIAIFSVMGFKASNDYGRCLDRNILSLINEFDLPELSISRDEYPSVLMYLNATQTARVAQLPLKTCHLEDFLDKSASGPGLAFIVFTEAVLHMPGASVWSVLFFGMLFTLGLSSMFGNMEGVITPLLDMGILPKGIPKEVMTAICFTLQSGGYWLEIFDSFAASLNLIIFAFMEVVGVIHIYGMKRFCDDIEWMTGRRPGLYWQVTWRVVSPMLLFGIFLSYIVLLIQTPPSYKAWNPQYEHFPSREEKFYPGWVQVTCVLLSFLPSLWVPGVALAQLLSQYKQRWKATHLESGLKLQESRGC

SEQ ID NO: 19 - protein, 578 aa, Mus musculus
MAQASGMDPLVDIEDERPKWDNKLQYLLSCIGFAVGLGNIWRFPYLCQTHGGGAFLIPYFIALVFEGIPLFYIELAIGQRLRRGSIGVWKTISPYLGGVGLGCFSVSFLVSLYYNTVLLWVLWFFLNSFQHPLPWSTCPLDLNRTGFVQECQSSGTVSYFWYRQTLNITSDISNTGTIQWKLFLCLVACWSTVYLCVIRGIESTGKVIYFTALFPYLVLTIFLIRGLTLPGATEGLIYLFTPNMKTLQNPRVWLDAATQIFFSLSLAFGGHIAFASYNPPRNNCEKDAVIIALVNSMTSLYASIAIFSVMGFKASNDYGRCLDRNILSLINEFDLPELSISRDEYPSVLMYLNATQTARVAQLPLKTCHLEDFLDKPTWKQISGARVLGEGCARLTSRVCEASVLPGVICFACFLSAICFTLQSGGYWLEIFDSFAASLNLIIFAFMEVVGVIHIYGMKRFCDDIEWMTGRRPGLYWQVTWRVVSPMLLFGIFLSYIVLLIQTPPSYKAWNPQYEHFPSREEKFYPGWVQVTCVLLSFLPSLWVPGVALAQLLSQYKQRWKATHLESGLKLQESRGCN

SEQ ID NO: 20 - protein, 552 aa, Mus musculus
MAQASGMDPLVDIEDERPKWDNKLQYLLSCIGFAVGLGNIWRFPYLCQTHGGGAFLIPYFIALVFEGIPLFYIELAIGQRLRRGSIGVWKTISPYLGGVGLGCFSVSFLVSLYYNTVLLWVLWFFLNSFQHPLPWSTCPLDLNRTGFVQECQSSGTVSYFWYRQTLNITSDISNTGTIQWKLFLCLVACWSTVYLCVIRGIESTGKVIYFTALFPYLVLTIFLIRGLTLPGATEGLIYLFTPNMKTLQNPRVWLDAATQIFFSLSLAFGGHIAFASYNPPRNNCEKDAVIIALVNSMTSLYASIAIFSVMGFKASNDYGRCLDRNILSLINEFDLPELSISRDEYPSVLMYLNATQTARVAQLPLKTCHLEDFLDKSASGPGLAFIVFTEAVLHMPGASVWSVLFFGMLFTLGLSSMFGNMEGVITPLLDMGILPKGIPKEVMTGVICFACFLSAICFTLQSGGYWLEIFDSFAASLNLIIFAFMEVVGVIHIYGMKRNIFPQERRSSTQAGCRSPVCSCPSCPHCGSLELLWLSYCPSTNRGGRLRIWKVV

SEQ ID NO: 21 - protein, 514 aa, Mus musculus
MAQASGMDPLVDIEDERPKWDNKLQYLLSCIGFAVGLGNIWRFPYLCQTHGGGAFLIPYFIALVFEGIPLFYIELAIGQRLRRGSIGVWKTISPYLGGVGLGCFSVSFLVSLYYNTVLLWVLWFFLNSFQHPLPWSTCPLDLNRTGFVQECQSSGTVSYFWYRQTLNITSDISNTGTIQWKLFLCLVACWSTVYLCVIRGIESTGKVIYFTALFPYLVLTIFLIRGLTLPGATEGLIYLFTPNMKTLQNPRVWLDAATQIFFSLSLAFGGHIAFASYNPPRNNCEKDAVIIALVNSMTSLYASIAIFSVMGFKASNDYGRCLDRNILSLINEFDLPELSISRDEYPSVLMYLNATQTARVAQLPLKTCHLEDFLDKPTWKQISGARVLGEGCARLTSRVCEASVLPGVICFACFLSAICFTLQSGGYWLEIFDSFAASLNLIIFAFMEVVGVIHIYGMKRNIFPQERRSSTQAGCRSPVCSCPSCPHCGSLELLWLSYCPSTNRGGRLRIWKVV

SEQ ID NO: 22 - protein, 423 aa, Mus musculus
MAQASGMDPLVDIEDERPKWDNKLQYLLSCIGFAVGLGNIWRFPYLCQTHGGGAFLIPYFIALVFEGIPLFYIELAIGQRLRRGSIGVWKTISPYLGGVGLGCFSVSFLVSLYYNTVLLWVLWFFLNSFQHPLPWSTCPLDLNRTGFVQECQSSGTVSYFWYRQTLNITSDISNTGTIQWKLFLCLVACWSTVYLCVIRGIESTGKVIYFTALFPYLVLTIFLIRGLTLPGATEGLIYLFTPNMKTLQNPRVWLDAATQIFFSLSLAFGGHIAFASYNPPRNNCEKDAVIIALVNSMTSLYASIAIFSVMGFKASNDYGRCLDRNILSLINEFDLPELSISRDEYPSVLMYLNATQTARVAQLPLKTCHLEDFLDKVLLCGLCSSLGCCLPWVCPPCLGTWRVSLHHYWTWGSYPKVYPRRSP

SEQ ID NO: 23 - protein, 615 aa, Rattus norvegicus
MAQASGMDPLVDIEDERPKWDNKLQYLLSCIGFAVGLGNIWRFPYLCHTHGGGAFLIPYFIALVFEGIPLFYIELAIGQRLRRGSIGVWKTISPYLGGVGLGCFSVSFLVSLYYNTILLWVLWFFLNSFQHPLPWSTCPLDLNRTGFVQECQSSGTVSYFWYRQTLNITSDISNTGTIQWKLFLCLVACWTTVYLCVIRGIESTGKVIYFTALFPYLVLTIFLIRGLTLPGATEGLTYLFTPNMKILQNSRVWLDAATQIFFSLSLAFGGHIAFASYNQPRNNCEKDAVTIALVNSMTSLYASITIFSIMGFKASNDYGRCLDRNILSLINEFDFPELSISRDEYPSVLMYLNATQPERVARLPLKTCHLEDFLDKSASGPGLAFIVFTEAVLHMPGASVWSVLFFGMLFTLGLSSMFGNMEGVITPLFDMGILPKGVPKETMTGVVCFICFLSAICFTLQSGSYWLEIFDSFAASLNLIIFAFMEVVGVIHVYGIKRFCDDIEWMTGRRPSLYWQVTWRVVSPMLLFGIFLSYIVLLAQSSPSYKAWNPQYEHFPSREEKLYPGWVQVTCVLLSFLPSLWVPGIALAQLLFQYRQRWKNTHLESALKPQESRGC

SEQ ID NO: 24 - protein, 734 aa, Rattus norvegicus
  MISC_FEATURE (731)..(731): wherein “X” equals any amino acid.
IPPKNSKVVKRDLDDDVIESVKDLLSNEDSVEDVSKKSELIVDVQEEKDTDAEDGSEVDDERPAWNSKLQYILAQVGFSVGLGNVWRFPYLCQKNGGGAYLLPYLILLLVIGIPLFFLELSVGQRIRRGSIGVWNYISPKLGGIGFASCVVCYFVALYYNVIIGWTLFYFSQSFQQPLPWDQCPLVKNASHTYIEPECEKSSATTYYWYREALAISSSISESGGLNWKMTGCLLAAWVMVCLAMIKGIQSSGKIMYFSSLFPYVVLICFLIRSLLLNGSIDGIRHMFTPKLEMMLEPKVWREAATQVFFALGLGFGGVIAFSSYNKRDNNCHFDAVLVSFINFFTSVLATLVVFAVLGFKANIVNEKCISQNSEMILKLLKTGNVSWDVIPRHINLSAVTAEDYHVVYDIIQKVKEEEFAVLHLKACQIEDELNKAVQGTGLAFIAFTEAMTHFPASPFWSVMFFLMLINLGLGSMFGTIEGIITPVVDTFKVRKEILTVICCLLAFCIGLMFVQRSGNYFVTMFDDYSATLPLLIVVILENIAVSFVYGIDKFLEDLTDMLGFAPSKYYYYMWKYISPLMLVTLLIASIVNMGLSPPGYNAWIKEKASEEFLSYPMWGMVVCFSLMVLAILPVPVVFVIRRCNLIDDSSGNLASVTYKRGRVLKEPVNLDGDDASLIHGKIPSEMSSPNFGKNIYRKQSGSPTLDTAPNGRYGIGYLMADMPDMPESDLXXXX

SEQ ID NO: 25 - protein, 728 aa, Rattus norvegicus
  MISC_FEATURE (152)..(152): wherein “X” equals any amino acid.
IPNSKVVKRDLDDDVIESVKDLLSNEDSVEDVSKKSELIVDVQEEKDTDAEDGSEVDDERPAWNSKLQYILAQVGFSVGLGNVWRFPYLCQKNGGGAYLLPYLILLLVIGIPLFFLELSVGQRIRRGSIGVWNYISPKLGGIGFASCVVCYXVALYYNVIIGWTLFYFSQSFQQPLPWDQCPLVKNASHTYIEXECEKSSATTYYWYREALAISSSISESGGLNWKMTGCLLAXWVMVCLAMIKGIQSSGKIMYFSSLFPYVVLICFLIRSLLLNGSIDGIRHMFTPKLEMMLEPKVWREAATQVFFALGLGFGGVIAFSSYNKRDNNCHFDAVLVSFINFFTSVLATLVVFAVLGFKANIVNEKCISQNSEMILKLLXTGNVSWDVIPRHINLSAVTAEDYHVVYDIIQKVKEEEFAVLHLKACQIEDELNKAVQGTGLAFIAFTEAMTHFPASPFWSVMFFLMLINLGLGSMFGTIEGIITPVVDTFKVRKEILTVICCLLAFCIGLMFVQRSGNYFVTMFDDYSATLPLLIVVILENIAVSFVYGIDKFLEDLTDMLGFAPSKYYYYMWKYISPLMLVTLLIASIVNMGLSPPGYNAWIKEKASEEFLSYPMWGMVVCFSLMVLAILPVPVVFVIRRCNLIDDSSGNLASVTYKRGRVLKEPVNLDGDDASLIHGKIPSEMSSPNFGKNIYRKQSGSPTLDTAPNGRYGIGYLMADMPDMPESDL

SEQ ID NO: 26 - protein, 616 aa, Rattus norvegicus
MRLAIKRRASRGQRPGPDEKRARDMEKARPQWGNPLQFVFACISYAVGLGNVWRFPYLCQMYGGGSFLVPYLIMLIVEGMPLLYLELAVGQRMRQGSIGAWRTISPYLSGVGVASVVVSFFLSMYYNVINAWGFWYLFHSFQDPLPWSVCPLNSNRTGYDEECEKASSTQYFWYRKTLNISPSIQENGGVQWEPALCLTLAWLMVYLCILRGTESTGKVVYFTALMPYCVLIIYLVRGLTLHGATNGLMYMFTPKIEQLANPKAWINAATQIFFSLGLGFGSLIAFASYNEPSNDCQKHAVIVSVINSSTSIFASIVTFSIYGFKATFNYENCLNKVILLLTNSFDLEDGFLTASNLEEVKDYLASTYPNKYSEVFPHIRNCSLESELNTAVQGTGLAFIVYAEAIKNMEVSQLWSVLYFFMLLMLGMGSMLGNTAAILTPLTDSKVISSYLPKEAISGLVCLINCAVGMVFTMEAGNYWFDIFNDYAATLSLLLIVLVETIAVCYVYGLRRFESDLRAMTGRPLNWYWKAMWAFVSPLLIIGLFIFYLSDYILTGTLQYQAWDATQGQLVTKDYPPHALAVIGLLVASSTMCIPLVALGTFIRNRLKRGGSSPVA

SEQ ID NO: 27 - protein, 265 aa, Bos taurus
MPKNSKVVKRELDDEVIESVKDLLSNEDSADDAFKKSELIVDVPEEKDTDVVERSEVKDARPAWNSKLQYILAQVGFSVGLGNVWRFPYLCQKNGGGAYLLPYLILLLVIGIPLFFLELSVGQRIRRGSIGVWNYISPQLGGIGFASCVVCFFVALYYNVIIGWSLFYFSQSFQQPLPWDQCPLVKNASHTFVEPECEKSSATTYYWYREALNISTSISESGGLNWKMTICLLAAWVVVCLAMIKGIQSSGKVSMSEPLLLLLNI

SEQ ID NO: 28 - protein, 729 aa, Bos taurus
MPKNSKVVKRELDDEVIESVKDLLSNEDSADDAFKKSELIVDVPEEKDTDVVERSEVKDARPAWNSKLQYILAQVGFSVGLGNVWRFPYLCQKNGGGAYLLPYLILLLVIGIPLFFLELSVGQRIRRGSIGVWNYISPQLGGIGFASCVVCFFVALYYNVIIGWSLFYFSQSFQQPLPWDQCPLVKNASHTFVEPECEKSSATTYYWYREALNISTSISESGGLNWKMTICLLAAWVVVCLAMIKGIQSSGKIMYFSSLFPYVVLICFLIRALLLNGSVDGIRHMFTPELEIMLEPKVWREAAAQVFFALGLGFGGVIAFSSYNKRDNNCHFDAVLVSFINFFTSILATLVVFAVLGFKANVINEKCIAENSEMIIKLVKMGNISQDIIPHHINFSAITAEDYDLIYDIIQKVKEEEFPALHLNACQIEDELNKAVQGTGLAFIAFTEAMTHFPASPFWSVMFFLMLVNLGLGSMFGTIEGIITPVVDTFKVRKEILTVICCLLAFCIGLIFVQRSGNYFVTMFDDYSATLPLLIVVILENIAVSFVYGIDKFMEDLKDMLGFTPNRYYYYMWKYISPLMLLSLLIASIVNMGLSPPGYNAWMEDKASEKFLSYPTWGMVICISLMVLAILPIPVVFIIRRCNLIDDSSGNLASVTYKRGRVLKEPVNLEGDDASLIHGKISSEMSSPNFGKNIYRKQSGSPTLDTAPNGRYGIGYLMADMPDMPESDL

SEQ ID NO: 29 - protein, 741 aa, Homo sapiens
MKTEAQPSTSLLANTSWTGTVISDSVPGSQTWEDKGSLTRSATSWTSEAQVSAARVAEAQARTSQPKQISVLEALTASALNQKPTHEKVQMTEKKESEVLLARPFWSSKTEYILAQVGFSMKPSCLWRFAYLWLNSGGCSFAAIYIFMLFLVGVPLLFLEMAAGQSMRQGGMGVWKIIAPRIGGVGYSSFMVCFILGLYFNVVNSWIIFYMSQSFQFPVPWEKCPLTMNSSGFDPECERTTPSIYFWYQQALKASDRIEDGGSPVYSLVLPFFLCWCLVGAFMINGLKSTGKVIYVLVLLPCFIIVGGFFIRTLLLEGAKFGLQQLVVAKISDVYNMSVWSLAGGQVLSNTGIGLGSVASLASYMPQSNNCLSDAFLVSVINLLTLLVFTSFNFCVLGFWATVITHRCCERNAEILLKLINLGKLPPDAKPPVNLLYNPTSIYNAWLSGLPQHIKSMVLREVTECNIETQFLKASEGPKFAFLSFVEAMSFLPPSVFWSFIFFLMLLAMGLSSAIGIMQGIITPLQDTFSFFRKHTKLLIVGVFLLMFVCGLFFTRPSGSYFIRLLSDYWIVFPIIVVVVFETMAVSWAYGARRFLADLTILLGHPISPIFGWLWPHLCPVVLLIIFVTMMVHLCMKPITYMSWDSSTSKEVLRPYPPWALLLMITLFAIVILPIPAYFVYCRIHRIPFRPKSGDGPMTASTSLPLSHQLTPSKEVQKEEILQVDETKYPSTCNVTSGARG

SEQ ID NO: 30 - protein, 624 aa, Artificial Sequence
  NTT Pfam Model Sequence
RETWSNKMDFVMSCIGFAVGLGNVWRFPYLCYKNGGGAFLIPYFIMMIFCGIPLFFMELALGQYHRQGCITVWRRKILDKGKGICPMFKGIGYWMCVICFYINIYYNVIIAWALYYLFSSFTTNLPWAHCNHWWNTPNCMEHHWCNNSTNWSYPMCSSKNMTHWTLHRTSPVMEFWERHVLHIHESDGIHDPGNLRWELTLCLLLAWIVCYFCIWKGVKSGSGKVVWFTATFPYVMLMCLLIRGVTLPGAWDGIQFYLTPDWHKLLDPQVWIDAATQIFFSYGICFGCLIAFASYNKFHNNCYRDCIIVCFINCITSFLAGFVIFSILGFMANIMQEQGVPQNEMILYLTNVSWDVIPHINFSHVTTDYHMYDIISEVAESQFVLHLPCIEDELDKVQAGPGLAFIAYPEAMTMMPASPFWACLFFFMLIFLGLDSQFCCMEGIITALVDEFPHLWRKMRREWFIVCCCVMCFLIGLFMVTEGGMYWFQLFDYYWASGMCLLWVVFFECICIAWFYGIDRFCDDIQEMMGFRPCWWWRWCWKFVSPCFCLFIFIFSIVQYGVQPLTYNNWIKEAEHYVYPWWAMWVGWWMALSSMICIPCYMIYRFCRTEGDTLWERLQYATTP

SEQ ID NO: 31 - DNA, 38 bp, Homo sapiens
gcagcagcggccgctacatcctggcccagattggcttc

SEQ ID NO: 32 - DNA, 37 bp, Homo sapiens
gcagcagtcgaccagctccgactcaggggtgctggcc

SEQ ID NO: 33 - DNA, 39 bp, Homo sapiens
gcagcagcggccgcatgccgaagaacagcaaagtgaccc

SEQ ID NO: 34 - DNA, 36 bp, Homo sapiens
gcagcagtcgacgaagtgccgcaggacgaacaccac

SEQ ID NO: 35 - protein, 22 aa, Homo sapiens
YILAQIGFSVGLGNIWRFPYLC

SEQ ID NO: 36 - protein, 23 aa, Homo sapiens
GAYLVPYLVLLIIIGIPLFFLEL

SEQ ID NO: 37 - protein, 25 aa, Homo sapiens
LGGIGFSSCIVCLFVGLYYNVIIGW

SEQ ID NO: 38 - protein, 20 aa, Homo sapiens
MTLCLLVAWSIVGMAVVKGI

SEQ ID NO: 39 - protein, 33 aa, Homo sapiens
VMYFSSLFPYVVLACFLVRGLLLRGAVDGILHM

SEQ ID NO: 40 - protein, 20 aa, Homo sapiens
AATQVFFALGLGFGGVIAFS

SEQ ID NO: 41 - protein, 20 aa, Homo sapiens
FINFFTSVLATLVVFAVLGF

SEQ ID NO: 42 - protein, 27 aa, Homo sapiens
VMFFLMLINLGLGSMIGTMAGITTPII

SEQ ID NO: 43 - protein, 22 aa, Homo sapiens
MFTVGCCVFAFLVGLLFVQRSG

SEQ ID NO: 44 - protein, 21 aa, Homo sapiens
YSATLPLTLIVILENIAVAWI

SEQ ID NO: 45 - protein, 21 aa, Homo sapiens
LCMAVLTTASIIQLGVTPPGY

SEQ ID NO: 46 - protein, 21 aa, Homo sapiens
MALLITLIVVATLPIPVVFVL

SEQ ID NO: 47 - protein, 20 aa, Homo sapiens
AVGQRIRRGSIGVWHYICPR

SEQ ID NO: 48 - protein, 63 aa, Homo sapiens
SIFYFFKSFQYPLPWSECPVVRNGSVAVVEAECEKSSATTYFWYREALDISDSISESGGLNWK

SEQ ID NO: 49 - protein, 16 aa, Homo sapiens
FTPKLDKMLDPQVWRE

SEQ ID NO: 50 - protein, 17 aa, Homo sapiens
SYNKQDNNCHFDAALVS

SEQ ID NO: 51 - protein, 102 aa, Homo sapiens
KANIMNEKCVVENAEKILGYLNTNVLSRDLIPPHVNFSHLTTKDYMEMYNVIMTVKEDQFSALGLDPCLLEDELDKSVQGTGLAFIAFTEAMTHFPASPFWS

SEQ ID NO: 52 - protein, 8 aa, Homo sapiens
DTFKVPKE

SEQ ID NO: 53 - protein, 9 aa, Homo sapiens
NYFVTMFDD

SEQ ID NO: 54 - protein, 31 aa, Homo sapiens
YGTKKFMQELTEMLGFRPYRFYFYMWKFVSP

SEQ ID NO: 55 - protein, 19 aa, Homo sapiens
SAWIKEEAAERYLYFPNWA

SEQ ID NO: 56 - DNA, 42 bp, Homo sapiens
gtccccaagcttgcaccatgccgaagaacagcaaagtgaccc

SEQ ID NO: 57 - DNA, 51 bp, Homo sapiens
cgggatcctacttgtcgtcgtcgtccttgtagtccagctccgactcagggg

SEQ ID NO: 58 - DNA, 27 bp, Homo sapiens
cgggatcctacagctccgactcagggg

SEQ ID NO: 59 - DNA, 23 bp, Homo sapiens
caggtgcagctggtgcagtctgg

SEQ ID NO: 60 - DNA, 23 bp, Homo sapiens
caggtcaacttaagggagtctgg

SEQ ID NO: 61 - DNA, 23 bp, Homo sapiens
gaggtgcagctggtggagtctgg

SEQ ID NO: 62 - DNA, 23 bp, Homo sapiens
caggtgcagctgcaggagtcggg

SEQ ID NO: 63 - DNA, 23 bp, Homo sapiens
gaggtgcagctgttgcagtctgc

SEQ ID NO: 64 - DNA, 23 bp, Homo sapiens
caggtacagctgcagcagtcagg

SEQ ID NO: 65 - DNA, 24 bp, Homo sapiens
tgaggagacggtgaccagggtgcc

SEQ ID NO: 66 - DNA, 24 bp, Homo sapiens
tgaagagacggtgaccattgtccc

SEQ ID NO: 67 - DNA, 24 bp, Homo sapiens
tgaggagacggtgaccagggttcc

SEQ ID NO: 68 - DNA, 24 bp, Homo sapiens
tgaggagacggtgaccgtggtccc

SEQ ID NO: 69 - DNA, 23 bp, Homo sapiens
gacatccagatgacccagtctcc

SEQ ID NO: 70 - DNA, 23 bp, Homo sapiens
gatgttgtgatgactcagtctcc

SEQ ID NO: 71 - DNA, 23 bp, Homo sapiens
gatattgtgatgactcagtctcc

SEQ ID NO: 72 - DNA, 23 bp, Homo sapiens
gaaattgtgttgacgcagtctcc

SEQ ID NO: 73 - DNA, 23 bp, Homo sapiens
gacatcgtgatgacccagtctcc

SEQ ID NO: 74 - DNA, 23 bp, Homo sapiens
gaaacgacactcacgcagtctcc

SEQ ID NO: 75 - DNA, 23 bp, Homo sapiens
gaaattgtgctgactcagtctcc

SEQ ID NO: 76 - DNA, 23 bp, Homo sapiens
cagtctgtgttgacgcagccgcc

SEQ ID NO: 77 - DNA, 23 bp, Homo sapiens
cagtctgccctgactcagcctgc

SEQ ID NO: 78 - DNA, 23 bp, Homo sapiens
tcctatgtgctgactcagccacc

SEQ ID NO: 79 - DNA, 23 bp, Homo sapiens
tcttctgagctgactcaggaccc

SEQ ID NO: 80 - DNA, 23 bp, Homo sapiens
cacgttatactgactcaaccgcc

SEQ ID NO: 81 - DNA, 23 bp, Homo sapiens
caggctgtgctcactcagccgtc

SEQ ID NO: 82 - DNA, 23 bp, Homo sapiens
aattttatgctgactcagcccca

SEQ ID NO: 83 - DNA, 24 bp, Homo sapiens
acgtttgatttccaccttggtccc

SEQ ID NO: 84 - DNA, 24 bp, Homo sapiens
acgtttgatctccagcttggtccc

SEQ ID NO: 85 - DNA, 24 bp, Homo sapiens
acgtttgatatccactttggtccc

SEQ ID NO: 86 - DNA, 24 bp, Homo sapiens
acgtttgatctccaccttggtccc

SEQ ID NO: 87 - DNA, 24 bp, Homo sapiens
acgtttaatctccagtcgtgtccc

SEQ ID NO: 88 - DNA, 23 bp, Homo sapiens
cagtctgtgttgacgcagccgcc

SEQ ID NO: 89 - DNA, 23 bp, Homo sapiens
cagtctgccctgactcagcctgc

SEQ ID NO: 90 - DNA, 23 bp, Homo sapiens
tcctatgtgctgactcagccacc

SEQ ID NO: 91 - DNA, 23 bp, Homo sapiens
tcttctgagctgactcaggaccc

SEQ ID NO: 92 - DNA, 23 bp, Homo sapiens
cacgttatactgactcaaccgcc

SEQ ID NO: 93 - DNA, 23 bp, Homo sapiens
caggctgtgctcactcagccgtc

SEQ ID NO: 94 - DNA, 23 bp, Homo sapiens
aattttatgctgactcagcccca

SEQ ID NO: 95 - DNA, 21 bp, Homo sapiens
caccctctccgtgtcctacaa

SEQ ID NO: 96 - DNA, 22 bp, Homo sapiens
tctcatcgttctcctccaggtt

SEQ ID NO: 97 - DNA, 25 bp, Homo sapiens
agatgtccttcatcatgcggccctt

SEQ ID NO: 98 - protein, 28 aa, Homo sapiens
VWREAATQVFFALGLGFGGVIAFSSYNK

SEQ ID NO: 99 - protein, 27 aa, Homo sapiens
LVSFINFFTSVLATLVVFAVLGFKANI

SEQ ID NO: 100 - protein, 83 aa, Homo sapiens
EEELDTEDRPAWNSKLQYILAQIGFSVGLGNIWRFPYLCQKNGGGAYLVPYLVLLIIIGIPLFFLELAVGQRIRRGSIGVWHY

SEQ ID NO: 101 - protein, 29 aa, Homo sapiens
CPRLGGIGFSSCIVCLFVGLYYNVIIGWS

SEQ ID NO: 102 - protein, 18 aa, Homo sapiens
FYFFKSFQYPLPWSECPV

SEQ ID NO: 103 - protein, 20 aa, Homo sapiens
ECEKSSATTYFWYREALDIS

SEQ ID NO: 104 - protein, 15 aa, Homo sapiens
SISESGGLNWKMTLC

SEQ ID NO: 105 - protein, 16 aa, Homo sapiens
IVGMAVVKGIQSSGKV

SEQ ID NO: 106 - protein, 40 aa, Homo sapiens
VQGTGLAFIAFTEAMTHFPASPFWSVMFFLMLINLGLGSM

SEQ ID NO: 107 - protein, 16 aa, Homo sapiens
VCLFVGLYYNVIIGWS

SEQ ID NO: 108 - protein, 18 aa, Homo sapiens
FYFFKSFQYPLPWSECPV

SEQ ID NO: 109 - protein, 20 aa, Homo sapiens
ECEKSSATTYFWYREALDIS

SEQ ID NO: 110 - protein, 107 aa, Homo sapiens
SISESGGLNWKMTLCLLVAWSIVGMAVVKGIQSSGKVMYFSSLFPYVVLACFLVRGLLLRGAVDGILHMFTPKLDKMLDPQVWREAATQVFFALGLGFGGVIAFSSY

SEQ ID NO: 111 - protein, 50 aa, Homo sapiens
KQDNNCHFDAALVSFINFFTSVLATLVVFAVLGFKANIMNEKCVVENAEK

SEQ ID NO: 112 - protein, 34 aa, Homo sapiens
DNNCHFDALVSFINFFTSVLATLVVFAVLGFKAN

SEQ ID NO: 113 - protein, 32 aa, Homo sapiens
VQGTGLAFIAFTEAMTHFPASPFWSVMFFLML

SEQ ID NO: 114 - protein, 21 aa, Homo sapiens
AVAWIYGTKKFMQELTEMLGF

SEQ ID NO: 115 - protein, 30 aa, Homo sapiens
PYRFYFYMWKFVSPLCMAVLTTASIIQLGV

SEQ ID NO: 116 - protein, 20 aa, Homo sapiens
PPGYSAWIKEEAAERYLYFP

SEQ ID NO: 117 - protein, 66 aa, Homo sapiens
LRHFHLLSDGSNTLSVSYKKGRMMKDISNLEENDETRFILSKVPSEAPSPMPTHRSYLGPGSTSPL

SEQ ID NO: 118 - protein, 20 aa, Homo sapiens
NPNGRYGSGYLLASTPESEL

SEQ ID NO: 119 - protein, 30 aa, Homo sapiens; identical to SEQ ID NO: 115

SEQ ID NO: 120 - protein, 31 aa, Homo sapiens
PPGYSAWIKEEAAERYLYFPNWAMALLITLI

SEQ ID NO: 121 - protein, 34 aa, Homo sapiens
LRHFHLLSDGSNTLSVSYKKGRMMKDISNLEEND

SEQ ID NO: 122 - protein, 31 aa, Homo sapiens
TRFILSKVPSEAPSPMPTHRSYLGPGSTSPL

SEQ ID NO: 123 - protein, 14 aa, Homo sapiens
RPAWNSKLQYILAQ

SEQ ID NO: 124 - protein, 16 aa, Homo sapiens
WRFPYLCQKNGGGAYL

SEQ ID NO: 125 - protein, 35 aa, Homo sapiens
LAFIAFTEAMTHFPASPFWSVMFFLMLINLGLGSM

SEQ ID NO: 126 - protein, 22 aa, Homo sapiens
FVQRSGNYFVTMFDDYSATLPL

SEQ ID NO: 127 - protein, 25 aa, Homo sapiens
TGLAFIAFTEAMTHFPASPFWSVMF

SEQ ID NO: 128 - protein, 29 aa, Homo sapiens
EHVTESVADLLALEEPVDYKQSVLNVAGE

SEQ ID NO: 129 - protein, 83 aa, Homo sapiens; identical to SEQ ID NO: 100

SEQ ID NO: 130 - protein, 26 aa, Homo sapiens
LGGIGFSSCIVCLFVGLYYNVIIGWS

SEQ ID NO: 131 - protein, 18 aa, Homo sapiens
FYFFKSFQYPLPWSECPV

SEQ ID NO: 132 - protein, 39 aa, Homo sapiens
PYRFYFYMWKFVSPLCMAVLTTASIIQLGVTPPGYSAWI

SEQ ID NO: 133 - protein, 91 aa, Homo sapiens
LPIPVVFVLRHFHLLSDGSNTLSVSYKKGRMMKDISNLEENDETRFILSKVPSEAPSPMPTHRSYLGPGSTSPLETSGNPNGRYGSGYLLA

SEQ ID NO: 134 - protein, 70 aa, Homo sapiens
EEELDTEDRPAWNSKLQYILAQIGFSVGLGNIWRFPYLCQKNGGGAYLVPYLVLLIIIGIPLFFLELAVG

SEQ ID NO: 135 - protein, 128 aa, Homo sapiens
IVGMAVVKGIQSSGKVMYFSSLFPYVVLACFLVRGLLLRGAVDGILHMFTPKLDKMLDPQVWREAATQVFFALGLGFGGVIAFSSYNKQDNNCHFDAALVSFINFFTSVLATLVVFAVLGFKANIMNE